(19) United States
(12) Patent Application Publication
(10) Pub. No.: US 2024/0200067 A1
(43) Pub. Date: Jun. 20, 2024

(54) Compositions for Modulating C9ORF72 Expression

(71) Applicant: Ionis Pharmaceuticals, Inc., Carlsbad, CA (US)

(72) Inventors: C. Frank Bennett, Carlsbad, CA (US); Susan M. Freier, San Diego, CA (US); Frank Rigo, Carlsbad, CA (US); Eric E. Swayze, Encinitas, CA (US)

(73) Assignee: Ionis Pharmaceuticals, Inc., Carlsbad, CA (US)

(21) Appl. No.: 18/330,084

(22) Filed: Jun. 6, 2023

Related U.S. Application Data
(63) Continuation of application No. 16/546,283, filed on Aug. 20, 2019, now abandoned.
Application No. 16/546,283 is a continuation of application No. 14/436,024, filed on Apr. 15, 2015, now Pat. No. 10,443,052.
(60) Provisional application No. 61/714,132, filed on Oct. 15, 2012.

(51) Int. Cl. C12N 15/113

(52) U.S. Cl. CPC C12N 15/113; C12N 2310/11; C12N 2310/313; C12N 2310/315; C12N 2310/322; C12N 2310/3233; C12N 2310/3341; C12N 2310/341; C12N 2310/346

(57) ABSTRACT

Disclosed herein are compositions and methods for reducing expression of C9ORF72 mRNA and protein in an animal with C9ORF72 specific inhibitors. Such methods are useful to treat, prevent, or ameliorate neurodegenerative diseases in an individual in need thereof. Such C9ORF72 specific inhibitors include antisense compounds. Examples of neurodegenerative diseases that can be treated, prevented, and ameliorated with the administration C9ORF72 specific inhibitors include amyotrophic lateral sclerosis (ALS), frontotemporal dementia (FTD), corticalbasal degeneration syndrome (CBD), atypical Parkinsonian syndrome, and olivopontocerellar degeneration (OPCD).

### CROSS-REFERENCE TO RELATED APPLICATIONS

This application is a continuation of U.S. application Ser. No. 16/546,283, filed Aug. 20, 2019, which is a continuation of U.S. application Ser. No. 14/436,024, filed Apr. 15, 2015, now U.S. Pat. No. 10,443,052, which is a national stage application under 35 U.S.C. § 371 of International Patent Application No. PCT/US2013/065073, filed Oct. 15, 2013, which claims the benefit of priority of U.S. Provisional Application No. 61/714,132, filed Oct. 15, 2012, each of which is incorporated by reference herein in its entirety for any purpose.

### SEQUENCE LISTING

The present application contains a Sequence Listing which has been submitted electronically in XML format. Said XML copy, created on May 30, 2023, is named “BIOL0211SEQ.xml” and is 415,802 bytes in size. The information in the electronic format of the sequence listing is incorporated herein by reference in its entirety.

### FIELD

Provided are compositions and methods for reducing expression of C9ORF72 mRNA and protein in an animal. Such methods are useful to treat, prevent, or ameliorate neurodegenerative diseases, including amyotrophic lateral sclerosis (ALS), frontotemporal dementia (FTD), corticalbasal degeneration syndrome (CBD), atypical Parkinsonian syndrome, and olivopontocerellar degeneration (OPCD).

### BACKGROUND

Amyotrophic lateral sclerosis (ALS) is a fatal neurodegenerative disease characterized clinically by progressive paralysis leading to death from respiratory failure, typically within two to three years of symptom onset (Rowland and Shneider, N. Engl. J. Med., 2001, 344, 1688-1700). ALS is the third most common neurodegenerative disease in the Western world (Hirtz et al., Neurology, 2007, 68, 326-337), and there are currently no effective therapies. Approximately 10% of cases are familial in nature, whereas the bulk of patients diagnosed with the disease are classified as sporadic as they appear to occur randomly throughout the population (Chio et al., Neurology, 2008, 70, 533-537). There is growing recognition, based on clinical, genetic, and epidemiological data, that ALS and frontotemporal dementia (FTD) represent an overlapping continuum of disease, characterized pathologically by the presence of TDP-43 positive inclusions throughout the central nervous system (Lillo and Hodges, J. Clin. Neurosci., 2009, 16, 1131-1135; Neumann et al., Science, 2006, 314, 130-133).

To date, a number of genes have been discovered as causative for classical familial ALS, for example, SOD1, TARDBP, FUS, OPTN, and VCP (Johnson et al., Neuron, 2010, 68, 857-864; Kwiatkowski et al., Science, 2009, 323, 1205-1208; Maruyama et al., Nature, 2010, 465, 223-226; Rosen et al., Nature, 1993, 362, 59-62; Sreedharan et al., Science, 2008, 319, 1668-1672; Vance et al., Brain, 2009, 129, 868-876). Recently, linkage analysis of kindreds involving multiple cases of ALS, FTD, and ALS-FTD had suggested that there was an important locus for the disease on the short arm of chromosome 9 (Boxer et al., J. Neurol. Neurosurg. Psychiatry, 2011, 82, 196-203; Morita et al., Neurology, 2006, 66, 839-844; Pearson et al. J. Nerol., 2011, 258, 647-655; Vance et al., Brain, 2006, 129, 868-876). The chromosome 9p21ALS-FTD locus in the last major autosomal-dominant gene whose mutation is causative of ALS. The ALS-FTD causing mutation is a large hexanucleotide (GGGGCC) repeat expansion in the first intron of the C9ORF72 gene (Renton et al., Neuron, 2011, 72, 257-268; DeJesus-Hernandez et al., Neuron, 2011, 72, 245-256). A founder haplotype, covering the C9ORF72 gene, is present in the majority of cases linked to this region (Renton et al., Neuron, 2011, 72, 257-268). This locus on chromosome 9p21 accounts for nearly half of familial ALS and nearly one-quarter of all ALS cases in a cohort of 405 Finnish patients (Laaksovirta et al, Lancet Neurol., 2010, 9, 978-985).

A founder haplotype, covering the C9ORF72 gene, is present in the majority of cases linked to this region.

There are currently no effective therapies to treat such neurodegenerative diseases. Therefore, it is an object to provide compositions and methods for the treatment of such neurodegenerative diseases.

### SUMMARY

Provided herein are compositions and methods for modulating levels of C9ORF72 mRNA and protein in cells, tissues, and animals. In certain embodiments, C9ORF72 specific inhibitors modulate expression of C9ORF72 mRNA and protein. In certain embodiments, C9ORF72 specific inhibitors are nucleic acids, proteins, or small molecules.

In certain embodiments, modulation can occur in a cell or tissue. In certain embodiments, the cell or tissue is in an animal. In certain embodiments, the animal is a human. In certain embodiments, C90RF72 mRNA levels are reduced. In certain embodiments, C9ORF72 protein levels are reduced. In certain embodiments, certain C9ORF72 mRNA variants are preferentially reduced. In certain embodiments, the C9ORF72 mRNA variants preferentially reduced are variants containing intron 1. In certain embodiments, intron 1 contains a hexanucleotide repeat expansion. In certain embodiments, the hexanucleotide repeat expansion is associated with a C9ORF72 associated disease. In certain embodiments, the hexanucleotide repeat expansion is associated with a C9ORF72 hexanucleotide repeat expansion associated disease. In certain embodiments, the hexanucleotide repeat expansion comprises at least 30 GGGGCC repeats. In certain embodiments, the hexanucleotide repeat expansion is associated with nuclear foci. In certain embodiments, the compositions and methods described herein are useful for reducing C9ORF72 mRNA levels, C9ORF72 protein levels, and nuclear foci. Such reduction can occur in a time-dependent manner or in a dose-dependent manner.

Also provided are methods useful for preventing, treating, and ameliorating diseases, disorders, and conditions associated with C9ORF72. In certain embodiments, such diseases, disorders, and conditions associated with C9ORF72 are neurodegenerative diseases. In certain embodiments, the neurodegenerative disease is amyotrophic lateral sclerosis (ALS), frontotemporal dementia (FTD), corticalbasal degeneration syndrome (CBD), atypical Parkinsonian syndrome, and olivopontocerellar degeneration (OPCD).

Such diseases, disorders, and conditions can have one or more risk factors, causes, or outcomes in common. Certain risk factors and causes for development of a neurodegenerative disease, and, in particular, ALS and FTD, include genetic predisposition and older age. In certain embodiments, methods of treatment include administering a C9ORF72 specific inhibitor to an individual in need thereof. In certain embodiments, the C9ORF72 specific inhibitor is a nucleic acid. In certain embodiments, the nucleic acid is an antisense compound. In certain embodiments, the antisense compound is a single-stranded antisense oligonucleotide. In certain embodiments, the single-stranded antisense oligonucleotide is complementary to a C9ORF72 nucleic acid.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Additionally, as used herein, the use of “and” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this disclosure, including, but not limited to, patents, patent applications, published patent applications, articles, books, treatises, and GENBANK Accession Numbers and associated sequence information obtainable through databases such as National Center for Biotechnology Information (NCBI) and other data referred to throughout in the disclosure herein are hereby expressly incorporated by reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis.

Unless otherwise indicated, the following terms have the following meanings:
“2′-O-methoxyethyl group” (also 2′-MOE and 2′-OCH₂CH₂—OCH₃ and MOE) refers to an O-methoxy-ethyl modification of the 2′ position of a furanosyl ring. A 2′-O-methoxyethyl modified sugar is a modified sugar.
“2′-MOE nucleoside” (also 2′-O-methoxyethyl nucleoside) means a nucleoside comprising a 2′-O-methoxyethyl group.
“5-methylcytosine” means a cytosine modified with a methyl group attached to the 5′ position. A 5-methylcytosine is a modified nucleobase.

“About” means within +7% of a value. For example, if it is stated, “the compounds affected at least about 70% inhibition of C9ORF72”, it is implied that the C9ORF72 levels are inhibited within a range of 63% and 77%. “Administered concomitantly” refers to the co-administration of two pharmaceutical agents in any manner in which the pharmacological effects of both are manifest in the patient at the same time. Concomitant administration does not require that both pharmaceutical agents be administered in a single pharmaceutical composition, in the same dosage form, or by the same route of administration. The effects of both pharmaceutical agents need not manifest themselves at the same time. The effects need only be overlapping for a period of time and need not be coextensive.

“Administering” means providing a pharmaceutical agent to an animal, and includes, but is not limited to administering by a medical professional and self-administering.

“Amelioration” or “ameliorate” or “ameliorating” refers to a lessening of at least one indicator, sign, or symptom of a disease, disorder, or condition. The severity of indicators may be determined by subjective or objective measures, which are known to those skilled in the art.

“Animal” refers to a human or non-human animal, including, but not limited to, mice, rats, rabbits, dogs, cats, pigs, and non-human primates, including, but not limited to, monkeys and chimpanzees.

“Antibody” refers to a molecule characterized by reacting specifically with an antigen in some way, where the antibody and the antigen are each defined in terms of the other. Antibody may refer to a complete antibody molecule or any fragment or region thereof, such as the heavy chain, the light chain, Fab region, and Fc region.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, antisense oligonucleotides, siRNAs, shRNAs, ssRNAs, and occupancy-based compounds. Antisense mechanisms include, without limitation, RNase H mediated antisense; RNAi mechanisms, which utilize the RISC pathway and include, without limitation, siRNA, ssRNA and microRNA mechanisms; and occupancy based mechanisms, including, without limitation uniform modified oligonucleotides. Certain antisense compounds may act through more than one such mechanism and/or through additional mechanisms.

“Antisense inhibition” means reduction of target nucleic acid levels or target protein levels in the presence of an antisense compound complementary to a target nucleic acid compared to target nucleic acid levels or target protein levels in the absence of the antisense compound. Inhibition may be any means including RNase H degradation, such as with a gapmer, and steric blockage, such as with a uniformly modified oligonucleotide.

“Antisense oligonucleotide” means a single-stranded oligonucleotide having a nucleobase sequence that permits hybridization to a corresponding segment of a target nucleic acid.

“Bicyclic sugar” means a furanosyl ring modified by the bridging of two atoms. A bicyclic sugar is a modified sugar.

“Bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

“C90RF72 associated disease” means any disease associated with any C9ORF72 nucleic acid or expression product thereof. Such diseases may include a neurodegenerative disease. Such neurodegenerative diseases may include ALS and FTD.

“C90RF72 hexanucleotide repeat expansion associated disease” means any disease associated with a C9ORF72 nucleic acid containing a hexanucleotide repeat expansion. In certain embodiments, the hexanucleotide repeat expansion may comprise GGGGCC, GGGGGG, GGGGGC, or GGGGCG repeated at least 30 times. Such diseases may include a neurodegenerative disease. Such neurodegenerative diseases may include ALS and FTD.

“C9ORF72 nucleic acid” means any nucleic acid encoding C9ORF72. For example, in certain embodiments, a C9ORF72 nucleic acid includes a DNA sequence encoding C9ORF72, an RNA sequence transcribed from DNA encoding C9ORF72 (including genomic DNA comprising introns and exons), and an mRNA sequence encoding C9ORF72. “C9ORF72 mRNA” means an mRNA encoding a C9ORF72 protein.

“C90RF72 specific inhibitor” refers to any agent capable of specifically inhibiting the expression of C9ORF72 mRNA and/or C9ORF72 protein at the molecular level. For example,

C90RF72 specific inhibitors include nucleic acids (including antisense compounds), siRNAs, aptamers, antibodies, peptides, small molecules, and other agents capable of inhibiting the expression of C90RF72 mRNA and/or C9ORF72 protein. Similarly, in certain embodiments, C90RF72 specific inhibitors may affect other molecular processes in an animal.

“Cap structure” or “terminal cap moiety” means chemical modifications, which have been incorporated at either terminus of an antisense compound.

“cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′-CH(CH₃)—O—₂′ bridge.

“Chemically distinct region” refers to a region of an antisense compound that is in some way chemically different than another region of the same antisense compound. For example, a region having 2′-O-methoxyethyl nucleosides is chemically distinct from a region having nucleosides without 2′-O-methoxyethyl modifications.

“Chimeric antisense compound” means an antisense compound that has at least two chemically distinct regions.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses parallel or sequential administration.

“Complementarity” means the capacity for pairing between nucleobases of a first nucleic acid and a second nucleic acid.

“Contiguous nucleobases” means nucleobases immediately adjacent to each other.

“Diluent” means an ingredient in a composition that lacks pharmacological activity, but is pharmaceutically necessary or desirable. For example, the diluent in an injected composition may be a liquid, e.g. saline solution.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month. “Effective amount” means the amount of pharmaceutical agent sufficient to effectuate a desired physiological outcome in an individual in need of the pharmaceutical agent. The effective amount may vary among individuals depending on the health and physical condition of the individual to be treated, the taxonomic group of the individuals to be treated, the formulation of the composition, assessment of the individual's medical condition, and other relevant factors.

“Expression” means conversion of the information from a C9ORF72 gene into mRNA via transcription and then to protein via translation. Expression may result in a phenotypic manifestation of the C9ORF72 gene.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap” and the external regions may be referred to as the “wings.”

“Gap-narrowed” means a chimeric antisense compound having a gap segment of 9 or fewer contiguous 2′-deoxyribonucleosides positioned between and immediately adjacent to 5′ and 3′ wing segments having from 1 to 6 nucleosides.

“Gap-widened” means a chimeric antisense compound having a gap segment of 12 or more contiguous 2′-deoxyribonucleosides positioned between and immediately adjacent to 5′ and 3′ wing segments having from 1 to 6 nucleosides.

“Hexanucleotide repeat expansion” means a series of six bases (for example, GGGGCC, GGGGGG, GGGGCG, or GGGGGC) repeated at least twice. In certain embodiments, the hexanucleotide repeat expansion may be located in intron 1 of a C9ORF72 nucleic acid. In certain embodiments, a pathogenic hexanucleotide repeat expansion includes at least 30 repeats of GGGGCC, GGGGGG, GGGGCG, or GGGGGC in a C90RF72 nucleic acid and is associated with disease. In certain embodiments, the repeats are consecutive. In certain embodiments, the repeats are interrupted by 1 or more nucleobases. In certain embodiments, a wild-type hexanucleotide repeat expansion includes 23 or fewer repeats of GGGGCC, GGGGGG, GGGGCG, or GGGGGC in a C90RF72 nucleic acid. In certain embodiments, the repeats are consecutive. In certain embodiments, the repeats are interrupted by 1 or more nucleobases.

“Hybridization” means the annealing of complementary nucleic acid molecules. In certain embodiments, complementary nucleic acid molecules include an antisense compound and a target nucleic acid.

“Identifying an animal having a C9ORF72 associated disease” means identifying an animal having been diagnosed with a C9ORF72 associated disease or predisposed to develop a C9ORF72 associated disease. Individuals predisposed to develop a C9ORF72 associated disease include those having one or more risk factors for developing a C9ORF72 associated disease, including, having a personal or family history or genetic predisposition of one or more C9ORF72 associated diseases. Such identification may be accomplished by any method including evaluating an individual's medical history and standard clinical tests or assessments, such as genetic testing. “Immediately adjacent” means there are no intervening elements between the immediately adjacent elements.

“Individual” means a human or non-human animal selected for treatment or therapy.

“Inhibiting C9ORF72” means reducing expression of C9ORF72 mRNA and/or protein levels in the presence of a C9ORF72 specific inhibitor, including a C9ORF72 antisense oligonucleotide, as compared to expression of C9ORF72 mRNA and/or protein levels in the absence of a C9ORF72 specific inhibitor, such as a C9ORF72 antisense oligonucleotide.

“Internucleoside linkage” refers to the chemical bond between nucleosides.

“Linked nucleosides” means adjacent nucleosides which are bonded together.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e., a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising a modified internucleoside linkage, a modified sugar, or a modified nucleobase.

“Modified sugar” refers to a substitution or change from a natural sugar.

“Motif” means the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“Nucleic acid” refers to molecules composed of monomeric nucleotides. A nucleic acid includes ribonucleic acids (RNA), deoxyribonucleic acids (DNA), single-stranded nucleic acids, double-stranded nucleic acids, small interfering ribonucleic acids (siRNA), and microRNAs (miRNA).

“Nucleobase” means a heterocyclic moiety capable of pairing with a base of another nucleic acid.

“Nucleobase sequence” means the order of contiguous nucleobases independent of any sugar, linkage, or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system.

“Nucleotide” means a nucleoside having a phosphate group covalently linked to the sugar portion of the nucleoside.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g., intrathecal or intracerebroventricular administration.

“Peptide” means a molecule formed by linking at least two amino acids by amide bonds. Peptide refers to polypeptides and proteins.

“Pharmaceutical agent” means the substance or substances in a pharmaceutical composition that provide a therapeutic benefit when administered to an individual. For example, in certain embodiments an antisense oligonucleotide targeted to C9ORF72 is a pharmaceutical agent.

“Pharmaceutical composition” means a mixture of substances suitable for administering to an individual. For example, a pharmaceutical composition may comprise one or more pharmaceutical agents and a sterile aqueous solution.

“Pharmaceutically acceptable derivative” encompasses pharmaceutically acceptable salts, conjugates, prodrugs or isomers of the compounds described herein.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage (P═S) is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” or “preventing” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment.

“3′ target site” refers to the 3′-most nucleotide of a target segment.

“Therapeutically effective amount” means an amount of a pharmaceutical agent that provides a therapeutic benefit to an individual.

“Treat” or “treating” refers to administering a pharmaceutical composition to effect an alteration or improvement of a disease, disorder, or condition.

“Unmodified nucleotide” means a nucleotide composed of naturally occuring nucleobases, sugar moieties, and internucleoside linkages. In certain embodiments, an unmodified nucleotide is an RNA nucleotide (i.e. B-D-ribonucleosides) or a DNA nucleotide (i.e. B-D-deoxyribonucleoside).

### Certain Embodiments

Certain embodiments provide methods for decreasing C9ORF72 mRNA and protein expression.

Certain embodiments provide methods for the treatment, prevention, or amelioration of diseases, disorders, and conditions associated with C9ORF72 in an individual in need thereof. Also contemplated are methods for the preparation of a medicament for the treatment, prevention, or amelioration of a disease, disorder, or condition associated with C9ORF72. C9ORF72 associated diseases, disorders, and conditions include neurodegenerative diseases. In certain embodiments, the neurodegenerative disease may be ALS or FTD. In certain embodiments, the neurodegenerative disease may be familial or sporadic.

Certain embodiments provide for the use of a C9ORF72 specific inhibitor for treating, preventing, or ameliorating a C9ORF72 associated disease. Certain embodiments provide for the use of a C9ORF72 specific inhibitor for treating, preventing, or ameliorating a C9ORF72 hexanucleotide repeat expansion associated disease. In certain embodiments, the hexanucleotide repeat expansion may comprise GGGGCC, GGGGGG, GGGGGC, or GGGGCG. In certain embodiments, C90RF72 specific inhibitors are nucleic acids (including antisense compounds), peptides, antibodies, small molecules, and other agents capable of inhibiting the expression of C9ORF72 mRNA and/or C9ORF72 protein.

Described herein are compounds comprising a single-stranded antisense oligonucleotide complementary to a C9ORF72 nucleic acid or a C9ORF72 homolog nucleic acid.

In certain embodiments, the C9ORF72 nucleic acid is a human C9ORF72 nucleic acid.

In certain embodiments, the C9ORF72 nucleic acid contains a hexanucleotide repeat expansion.

In certain embodiments, the C9ORF72 nucleic acid does not contain a hexanucleotide repeat expansion.

In certain embodiments, the single-stranded antisense oligonucleotide is specifically hybridizable to a human C9ORF72 nucleic acid.

In certain embodiments, the single-stranded antisense oligonucleotide is at least 75%, at least 80%, at least 85%, at least 90%, at least 95%, or 100% complementary to an equal length portion of a human C9ORF72 nucleic acid.

In certain embodiments, the single-stranded antisense oligonucleotide is complementary to any of exon, an intron, the 5′ UTR, the 3′ UTR, a repeat region, a splice junction, an exon:exon splice junction, an exonic splicing silencer (ESS), an exonic splicing enhancer (ESE), exon 1a, exon 1b, exon 1c, exon 1d, exon le, exon 2, exon 3, exon 4, exon 5, exon 6, exon 7, exon 8, exon 9, exon 10, exon 11, intron 1, intron 2, intron 3, intron 4, intron 5, intron 6, intron 7, intron 8, intron 9, or intron 10 of a human C9ORF72 nucleic acid.

Described herein are compounds comprising a single-stranded antisense oligonucleotide consisting of 12 to 30 linked nucleosides and comprising a nucleobase sequence comprising at least 8, at least 9, at least 10, at least 11, at least 12, at least 13, at least 14, at least 15, at least 16, at least 17, at least 18, at least 19, or at least 20 contiguous nucleobases of SEQ ID NO: 30-369.

In certain embodiments, the single-stranded antisense oligonucleotide comprises at least one modification.

In certain embodiments, the single-stranded antisense oligonucleotide comprises at least one modified internucleoside linkage.

In certain embodiments, each internucleoside linkage of the single-stranded antisense oligonucleotide is a modified internucleoside linkage.

In certain embodiments, the modified internucleoside linkage is a phosphorothioate internucleoside linkage.

In certain embodiments, the single-stranded antisense oligonucleotide comprises at least one modified nucleoside.

In certain embodiments, the single-stranded antisense oligonucleotide comprises at least one modified nucleoside having a modified sugar.

In certain embodiments, the single-stranded antisense oligonucleotide comprises at least one modified nucleoside comprising a bicyclic sugar.

In certain embodiments, the bicyclic sugar comprises a 4′ to 2′ bridge selected from among: 4′-(CH₂)ₙ—O-2′ bridge, wherein n is 1 or 2; and 4′-CH₂—O—CH₂-2′.

In certain embodiments, the bicyclic sugar comprises a 4′-CH(CH₃)—O-2′ bridge.

In certain embodiments, the at least one modified nucleoside having a modified sugar comprises a non-bicyclic 2′-modified modified sugar moiety.

In certain embodiments, the 2′-modified sugar moiety comprises a 2′-O-methoxyethyl group.

In certain embodiments, the 2′-modified sugar moiety comprises a 2′-O-methyl group.

In certain embodiments, the at least one modified nucleoside having a modified sugar comprises a sugar surrogate.

In certain embodiments, the sugar surrogate is a morpholino.

In certain embodiments, the sugar surrogate is a peptide nucleic acid.

In certain embodiments, each nucleoside is modified.

In certain embodiments, the single-stranded antisense oligonucleotide comprises at least one modified nucleobase.

In certain embodiments, the modified nucleobase is a 5′-methylcytosine.

In certain embodiments, the single-stranded antisense oligonucleotide comprises:

a gap segment consisting of linked deoxynucleosides;
a 5′ wing segment consisting of linked nucleosides;
a 3′ wing segment consisting of linked nucleosides;
wherein the gap segment is positioned immediately adjacent to and between the 5′ wing segment and the 3′ wing segment and wherein each nucleoside of each wing segment comprises a modified sugar.

In certain embodiments, the single-stranded antisense oligonucleotide comprises:

a gap segment consisting of ten linked deoxynucleosides;
a 5′ wing segment consisting of five linked nucleosides;
a 3′ wing segment consisting of five linked nucleosides;
wherein the gap segment is positioned immediately adjacent and between the 5′ wing segment and the 3′ wing segment, wherein each nucleoside of each wing segment comprises a 2′-O— methoxyethyl sugar; and wherein each internucleoside linkage is a phosphorothioate linkage.

In certain embodiments, the single-stranded antisense oligonucleotide consists of 15 linked nucleosides.

In certain embodiments, the single-stranded antisense oligonucleotide consists of 16 linked nucleosides.

In certain embodiments, the single-stranded antisense oligonucleotide consists of 17 linked nucleosides.

In certain embodiments, the single-stranded antisense oligonucleotide consists of 18 linked nucleosides.

In certain embodiments, the single-stranded antisense oligonucleotide consists of 19 linked nucleosides.

In certain embodiments, the single-stranded antisense oligonucleotide consists of 20 linked nucleosides.

In certain embodiments, the single-stranded antisense oligonucleotide consists of 21 linked nucleosides.

In certain embodiments, the single-stranded antisense oligonucleotide consists of 22 linked nucleosides.

In certain embodiments, the single-stranded antisense oligonucleotide consists of 23 linked nucleosides.

In certain embodiments, the single-stranded antisense oligonucleotide consists of 24 linked nucleosides.

In certain embodiments, the single-stranded antisense oligonucleotide consists of 25 linked nucleosides.

Described herein are uses of the compound for the manufacture of a medicament for treating a neurodegenerative disease.

Provided herein are methods of preferentially inhibiting expression of mRNA transcripts containing a hexanucleotide repeat expansion by contacting a cell with an antisense oligonucleotide targeting upstream of exon 1B.

### Antisense Compounds

Oligomeric compounds include, but are not limited to, oligonucleotides, oligonucleosides, oligonucleotide analogs, oligonucleotide mimetics, antisense compounds, antisense oligonucleotides, and siRNAs. An oligomeric compound may be “antisense” to a target nucleic acid, meaning that is is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a C9ORF72 nucleic acid is 12 to 30 subunits in length. In other words, such antisense compounds are from 12 to 30 linked subunits. In certain embodiments, the antisense compound is 8 to 80, 12 to 50, 15 to 30, 18 to 24, 19 to 22, or 20 linked subunits. In certain embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleosides.

In certain embodiments antisense oligonucleotides targeted to a C9ORF72 nucleic acid may be shortened or truncated. For example, a single subunit may be deleted from the 5′ end (5′ truncation), or alternatively from the 3′ end (3′ truncation). A shortened or truncated antisense compound targeted to a C9ORF72 nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358,1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a C9ORF72 nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH₂)ₙ—O-2′ bridge, where n=1 or n=2 and 4′-CH₂—O—CH₂-2′). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent to each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers described herein include, but are not limited to, for example 5-10-5, 5-10-4, 4-10-4, 4-10-3, 3-10-3, 2-10-2, 5-9-5, 5-9-4, 4-9-5, 5-8-5, 5-8-4, 4-8-5, 5-7-5, 4-7-5, 5-7-4, or 4-7-4.

In certain embodiments, the antisense compound has a “wingmer” motif, having a wing-gap or gap-wing configuration, i.e. an X-Y or Y-Z configuration as described above for the gapmer configuration. Thus, wingmer configurations described herein include, but are not limited to, for example 5-10, 8-4, 4-12, 12-4, 3-14, 16-2, 18-1, 10-3, 2-10, 1-10, 8-2, 2-13, 5-13, 5-8, or 6-8.

In certain embodiments, antisense compounds targeted to a C9ORF72 nucleic acid possess a 5-10-5 gapmer motif.

In certain embodiments, antisense compounds targeted to a C9ORF72 nucleic acid possess a 5-10-4 gapmer motif.

In certain embodiments, antisense compounds targeted to a C9ORF72 nucleic acid possess a 4-10-4 gapmer motif.

In certain embodiments, antisense compounds targeted to a C9ORF72 nucleic acid possess a 4-10-3 gapmer motif.

In certain embodiments, antisense compounds targeted to a C9ORF72 nucleic acid possess a 5-9-5 gapmer motif.

In certain embodiments, an antisense compound targeted to a C9ORF72 nucleic acid has a gap-narrowed motif. In certain embodiments, a gap-narrowed antisense oligonucleotide targeted to a C90RF72 nucleic acid has a gap segment of 9, 8, 7, or 6 2′-deoxynucleotides positioned immediately adjacent to and between wing segments of 5, 4, 3, 2, or 1 chemically modified nucleosides. In certain embodiments, the chemical modification comprises a bicyclic sugar. In certain embodiments, the bicyclic sugar comprises a 4′ to 2′ bridge selected from among: 4′-(CH₂)ₙ—O-2′ bridge, wherein n is 1 or 2; and 4′-CH₂—O—CH₂-2′. In certain embodiments, the bicyclic sugar is comprises a 4′-CH(CH₃)—O-2′ bridge. In certain embodiments, the chemical modification comprises a non-bicyclic 2′-modified sugar moiety. In certain embodiments, the non-bicyclic 2′-modified sugar moiety comprises a 2′-O-methylethyl group or a 2′-O-methyl group.

In certain embodiments, an antisense compound targeted to a C9ORF72 nucleic acid is uniformly modified. In certain embodiments, the antisense compound comprises 12, 13, 14, 15, 16, 17, 18, 19, 20, 21, 22, 23, 24, or 25 nucleosides. In certain embodiments, each nucleosides is chemically modified. In certain embodiments, the chemical modification comprises a non-bicyclic 2′-modified sugar moiety. In certain embodiments, the 2′-modified sugar moiety comprises a 2′-O-methoxyethyl group. In certain embodiments, the 2′-modified sugar moiety comprises a 2′-O-methyl group. In certain embodiments, uniformly modified antisense compounds may target C9ORF72, or any portion thereof, such as a hexanucleotide repeat expansion. In certain embodiments, targeting the hexanucleotide repeat expansion with a unformily modified antisense compound reduces the repeat RNA by blocking the interaction with RNA binding proteins. In certain embodiments, this results in the toxic RNA being absent from foci and benig degraded instead.

### Target Nucleic Acids, Target Regions and Nucleotide Sequences

Nucleotide sequences that encode C9ORF72 include, without limitation, the following: the complement of GENBANK Accession No. NM_001256054.1 (incorporated herein as SEQ ID NO: 1), GENBANK Accession No. NT_008413.18 truncated from nucleobase 27535000 to 27565000 (incorporated herein as SEQ ID NO: 2), GENBANK Accession No. BQ068108.1 (incorporated herein as SEQ ID NO: 3), GENBANK Accession No. NM_018325.3 (incorporated herein as SEQ ID NO: 4), GENBANK Accession No. DN993522.1 (incorporated herein as SEQ ID NO: 5), GENBANK Accession No. NM_145005.5 (incorporated herein as SEQ ID NO: 6), GENBANK Accession No. DB079375.1 (incorporated herein as SEQ ID NO: 7), GENBANK Accession No. BU194591.1 (incorporated herein as SEQ ID NO: 8), Sequence Identifier 4141_014_A (incorporated herein as SEQ ID NO: 9), and Sequence Identifier 4008_73_A (incorporated herein as SEQ ID NO: 10).

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for C9ORF72 can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the preceding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize in a non-specific manner to sequences other than a selected target nucleic acid (i.e., non-target or off-target sequences).

There may be variation in activity (e.g., as defined by percent reduction of target nucleic acid levels) of the antisense compounds within a target region. In certain embodiments, reductions in C9ORF72 mRNA levels are indicative of inhibition of C9ORF72 expression. Reductions in levels of a C9ORF72 protein are also indicative of inhibition of target mRNA expression. Reduction in the presence of expanded C9ORF72 RNA foci are indicative of inhibition of C9ORF72 expression.

Further, phenotypic changes are indicative of inhibition of C9ORF72 expression. For example, improved motor function and respiration may be indicative of inhibition of C9ORF72 expression.

### Hybridization

In some embodiments, hybridization occurs between an antisense compound disclosed herein and a C9ORF72 nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a C9ORF72 nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a C9ORF72 nucleic acid).

Non-complementary nucleobases between an antisense compound and a C9ORF72 nucleic acid may be tolerated provided that the antisense compound remains able to specifically hybridize to a target nucleic acid. Moreover, an antisense compound may hybridize over one or more segments of a C9ORF72 nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a C9ORF72 nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park,

Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e., 100% complementary) to a target nucleic acid, or specified portion thereof. For example, an antisense compound may be fully complementary to a C9ORF72 nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e., linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a C9ORF72 nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a C9ORF72 nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 9 nucleobase portion of a target segment. In certain embodiments, the antisense compounds are complementary to at least a 10 nucleobase portion of a target segment. In certain embodiments, the antisense compounds, are complementary to at least an 11 nucleobase portion of a target segment. In certain embodiments, the antisense compounds, are complementary to at least a 12 nucleobase portion of a target segment. In certain embodiments, the antisense compounds, are complementary to at least a 13 nucleobase portion of a target segment. In certain embodiments, the antisense compounds, are complementary to at least a 14 nucleobase portion of a target segment. In certain embodiments, the antisense compounds, are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, an antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occuring internucleoside linkage of RNA and DNA is a 3′ to 5′ phosphodiester linkage. Antisense compounds having one or more modified, i.e. non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a C9ORF72 nucleic acid comprise one or more modified internucleoside linkages. In certain embodiments, the modified internucleoside linkages are interspersed throughout the antisense compound. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′, 2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O-C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), where each R₁, Rₘ and Rₙ is, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′-(CH₂)₂-O-2′ (ENA); 4′-CH(CH₃)—O-2′ and 4′-CH(CH₂OCH₃)-O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(—CH₂)-2′ (and analogs thereof see published International Application WO 2008/154401, published on Dec. 8, 2008).

Further reports related to bicyclic nucleosides can also be found in published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U. S. A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Serial Nos. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)=C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted Cs-Czalicyclic radical, halogen, OJ₁, NJ_{b 1}J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(—O)—H), substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[C(Rₐ)(R_{b})]ₙ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂—O—N(R)-2′ and 4′-CH₂—N(R)—O-2′- wherein each R is, independently, H, a protecting group or C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides include, but are not limited to, (A) α-L-methyleneoxy (4′-CH₂—O-2′) BNA , (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, and (F) methyl(methyleneoxy) (4′-CH(CH₃)—O-2′) BNA, (G) methylene-thio (4′-CH₂—S-2′) BNA, (H) methylene-amino (4′-CH₂—N(R)-2′) BNA, (I) methyl carbocyclic (4′-CH₂—CH(CH₃)-2′) BNA, and (J) propylene carbocyclic (4′-(CH₂)₃-2′) BNA as depicted below.

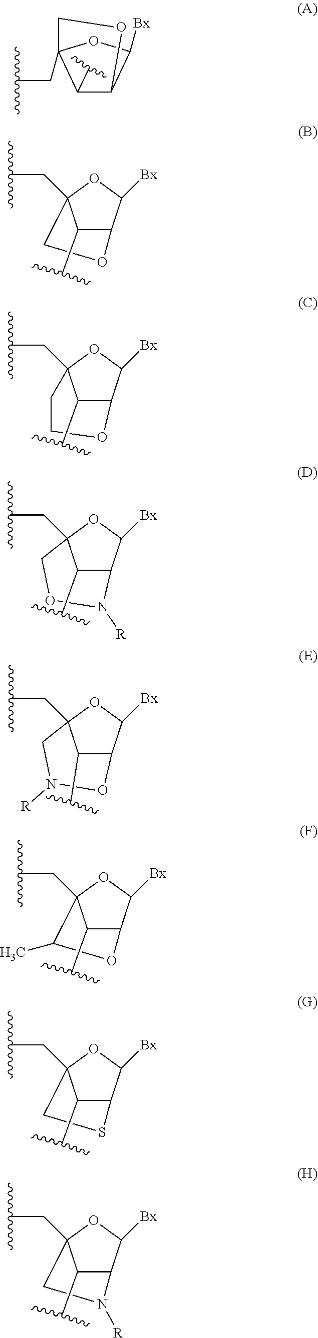

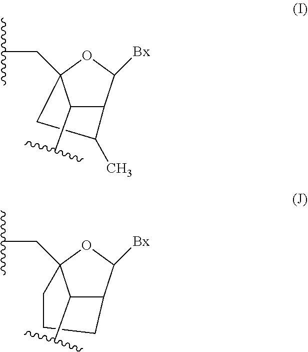

wherein Bx is the base moiety and R is independently H, a protecting group or C₁-C₁₂ alkyl.

In certain embodiments, bicyclic nucleosides are provided having Formula I:

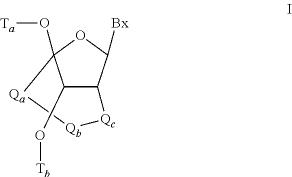

wherein:

Bx is a heterocyclic base moiety;

-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O—or —N(R_{c})—O—CH₂;

R_{c} is C₁-C₁₂ alkyl or an amino protecting group; and

Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides are provided having Formula II:

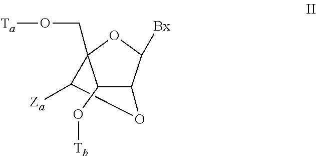

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJₑC(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides are provided having Formula III:

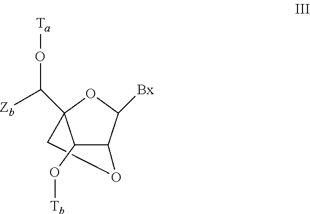

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(—O)—).

In certain embodiments, bicyclic nucleosides are provided having Formula IV:

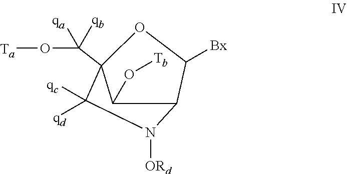

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxyl, substituted C₁-C₆ alkoxyl, acyl, substituted acyl, C₁-C₆ aminoalkyl or substituted C₁-C₆ aminoalkyl;

In certain embodiments, bicyclic nucleosides are provided having Formula V:

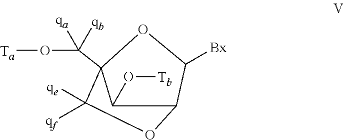

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are=C(q_{g})(qₕ);

q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides are provided having Formula VI:

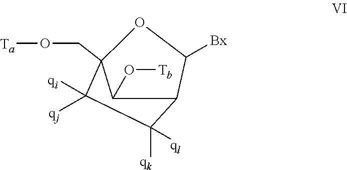

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

each qᵢ, qᵢ, qₖ and qᵢ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH—CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc., 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₃, and O(CH₂)ₙON[(CH₂)ₙCH₃]₂, where n and m are from 1 to about 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an antisense compound, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854), fluoro HNA (F-HNA) or those compounds having Formula VII:

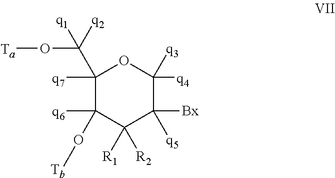

wherein independently for each of said at least one tetrahydropyran nucleoside analog of Formula VII:

Bx is a heterocyclic base moiety;

Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, substituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one of q₁, q₂, q₃, q₄, q₅, q₆ and q₇is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro. In certain embodiments, R₁ is fluoro and R₂ is H; R₁ is methoxy and R₂ is H, and R₁ is H and R₂ is methoxyethoxy.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′substituents, such as allyl, amino, azido, thio, O-allyl, O-C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modified nucleosides may further comprise other modifications, for example at other positions of the sugar and/or at the nucleobase.

As used herein, “2′-F” refers to a nucleoside comprising a sugar comprising a fluoro group at the 2′ position.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854). Such ring systems can undergo various additional substitutions to enhance activity.

Methods for the preparations of modified sugars are well known to those skilled in the art.

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

### Compositions and Methods for Formulating Pharmaceutical Compositions

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense compound targeted to a C9ORF72 nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, in one embodiment, employed in the methods described herein is a pharmaceutical composition comprising an antisense compound targeted to a C9ORF72 nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of C9ORF72 nucleic acids can be tested in vitro in a variety of cell types. Cell types used for such analyses are available from commercial vendors (e.g. American Type Culture Collection, Manassus, VA; Zen-Bio, Inc., Research Triangle Park, NC; Clonetics Corporation, Walkersville, MD) and are cultured according to the vendor's instructions using commercially available reagents (e.g. Invitrogen Life Technologies, Carlsbad, CA). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transfection reagent LIPOFECTIN (Invitrogen, Carlsbad, CA). Antisense oligonucleotides are mixed with LIPOFECTIN in OPTI-MEM 1 (Invitrogen, Carlsbad, CA) to achieve the desired final concentration of antisense oligonucleotide and a LIPOFECTIN concentration that typically ranges 2 to 12 ug/mL per 100 nM antisense oligonucleotide.

Another reagent used to introduce antisense oligonucleotides into cultured cells includes LIPOFECTAMINE (Invitrogen, Carlsbad, CA). Antisense oligonucleotide is mixed with LIPOFECTAMINE in OPTI-MEM 1 reduced serum medium (Invitrogen, Carlsbad, CA) to achieve the desired concentration of antisense oligonucleotide and a LIPOFECTAMINE concentration that typically ranges 2 to 12 ug/mL per 100 nM antisense oligonucleotide.

Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+ mRNA. Methods of RNA isolation are well known in the art. RNA is prepared using methods well known in the art, for example, using the TRIZOL Reagent (Invitrogen, Carlsbad, CA) according to the manufacturer's recommended protocols.

### Analysis of Inhibition of Target Levels or Expression

Inhibition of levels or expression of a C9ORF72 nucleic acid can be assayed in a variety of ways known in the art. For example, target nucleic acid levels can be quantitated by, e.g., Northern blot analysis, competitive polymerase chain reaction (PCR), or quantitaive real-time PCR. RNA analysis can be performed on total cellular RNA or poly(A)+ mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, CA and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, CA) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, CA). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, CA). Cyclophilin A expression is quantified by real time PCR, by being run simultaneously with the target, multiplexing, or separately. Total RNA is quantified using RIBOGREEN RNA quantification reagent (Invetrogen, Inc. Eugene, OR). Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a C9ORF72 nucleic acid. Methods for designing real-time PCR probes and primers are well known in the art, and may include the use of software such as PRIMER EXPRESS Software (Applied Biosystems, Foster City, CA).

### Analysis of Protein Levels

Antisense inhibition of C9ORF72 nucleic acids can be assessed by measuring C9ORF72 protein levels. Protein levels of C9ORF72 can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained from a variety of sources, such as the MSRS catalog of antibodies (Aerie Corporation, Birmingham, MI), or can be prepared via conventional monoclonal or polyclonal antibody generation methods well known in the art. Antibodies useful for the detection of mouse, rat, monkey, and human C9ORF72 are commercially available.

### In Vivo Testing of Antisense Compounds

Antisense compounds, for example, antisense oligonucleotides, are tested in animals to assess their ability to inhibit expression of C9ORF72 and produce phenotypic changes, such as, improved motor function and respiration. In certain embodiments, motor function is measured by rotarod, grip strength, pole climb, open field performance, balance beam, hindpaw footprint testing in the animal. In certain embodiments, respiration is measured by whole body plethysmograph, invasive resistance, and compliance measurements in the animal. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated from CNS tissue or CSF and changes in C9ORF72 nucleic acid expression are measured.

### Targeting (9ORF72

Antisense oligonucleotides described herein may hybridize to a C9ORF72 nucleic acid in any stage of RNA processing. For example, described herein are antisense oligonucleotides that are complementary to a pre-mRNA or a mature mRNA. Additionally, antisense oligonucleotides described herein may hybridize to any element of a C9ORF72 nucleic acid. For example, described herein are antisense oligonucleotides that are complementary to an exon, an intron, the 5′ UTR, the 3′ UTR, a repeat region, a hexanucleotide repeat expansion, a splice junction, an exon:exon splice junction, an exonic splicing silencer (ESS), an exonic splicing enhancer (ESE), exon 1a, exon 1b, exon 1c, exon 1d, exon 1e, exon 2, exon 3, exon 4, exon 5, exon 6, exon 7, exon 8, exon 9, exon 10, exon11, intron 1, intron 2, intron 3, intron 4, intron 5, intron 6, intron 7, intron 8, intron 9, or intron 10 of a C9ORF72 nucleic acid.

In certain embodiments, antisense oligonucleotides described herein hybridize to all variants of C9ORF72. In certain embodiments, the antisense oligonucleotides described herein selectively hybridize to certain variants of C9ORF72. In certain embodiments, the antisense oligonucleotides described herein selectively hybridize to variants of C9ORF72 containing a hexanucleotide repeat expansion. In certain embodiments, such variants of C9ORF72 containing a hexanucleotide repeat expansion include SEQ ID NO: 1-3 and 6-10. In certain embodiments, such hexanucleotide repeat expansion comprises at least 30 repeats of any of GGGGCC, GGGGGG, GGGGGC, or GGGGCG.

In certain embodiments, the antisense oligonucleotides described herein inhibit expression of all variants of C9ORF72. In certain embodiments, the antisense oligonucleotides described herein inhibit expression of all variants of C9ORF72 equally. In certain embodiments, the antisense oligonucleotides described herein preferentially inhibit expression of certain variants of C9ORF72. In certain embodiments, the antisense oligonucleotides described herein preferentially inhibit expression of variants of C9ORF72 containing a hexanucleotide repeat expansion. In certain embodiments, such variants of C9ORF72 containing a hexanucleotide repeat expansion include SEQ ID NO: 1-3 and 6-10. In certain embodiments, such hexanucleotide repeat expansion comprises at least 30 repeats of any of GGGGCC, GGGGGG, GGGGGC, or GGGGCG. In certain embodiments, the hexanucleotide repeat expansion forms nuclear foci. In certain embodiments, antisense oligonucleotides described herein are useful for reducing nuclear foci. Nuclear foci may be reduced in terms of percent of cells with foci as well as number of foci per cell.

Based on earlier studies directed to repeat expansions, it is not possible to predict if antisense oligonucleotides targeting C9ORF72 outside of the hexanucleotide repeat expansion would successfully inhibit expression of C9ORF72 for two reasons. First, the C9ORF72 repeat expansion is located in an intron and it is not known if the RNA in the foci contains only the repeats or also the flanking intronic sequence. For example, an earlier study on myotonic dystrophy type 2 (DM2), which is a disease caused by a CCTG expansion mutation in intron 1 of the ZNF9 gene, determined that large DM2 expansions did not prevent allele-specific pre-mRNA splicing, nuclear export of the transcripts, or steady-state mRNA or protein levels. The study further demonstrated that the ribonuclear inclusions found associated with the disease are enriched for the CCUG expansion, but not the flanking intronic sequences. These data suggest that the downstream molecular effects of the DM2 mutation may be triggered by the accumulation of CCUG repeat tract alone. Therefore, this study implies that targeting the CCUG repeat expansion alone would lead to amelioration of the disease, since targeting the flanking sequences, especially the region downstream of the repeat expansion, would not affect the formation of ribonuclear inclusions (Margolis et al. Hum. Mol. Genet., 2006, 15:1808-1815). Second, it is not known how fast intron 1 of C9ORF72, which contains the repeats, is excised and accumulates in foci. Thus, it is not possible to predict if targeting the pre-mRNA would result in elimination of the repeat RNA and foci.

### (9OFF72 Features

Antisense oligonucleotides described herein may hybridize to any C9ORF72 variant at any state of processing within any element of the C9ORF72 gene. For example, antisense oligonucleotides described herein may hybridize to an exon, an intron, the 5′ UTR, the 3′ UTR, a repeat region, a hexanucleotide repeat expansion, a splice junction, an exon:exon splice junction, an exonic splicing silencer (ESS), an exonic splicing enhancer (ESE), exon 1a, exon 1b, exon 1c, exon 1d, exon 1e, exon 2, exon 3, exon 4, exon 5, exon 6, exon 7, exon 8, exon 9, exon 10, exon 11, intron 1, intron 2, intron 3, intron 4, intron 5, intron 6, intron 7, intron 8, intron 9, or intron 10. For example, antisense oligonucleotides may target any of the exons characterized below in Tables 1-5 for the various C9ORF72 variants described below. Antisense oligonucleotides described herein may also target variants not characterized below and such variants are characterized in GENBANK. Moreover, antisense oligonucleotides described herein may also target elements other than exons and such elements are characterized in GENBANK.

TABLE 1
Functional Segments for NM_001256054.1 (SEQ ID NO: 1)

| | | | Start site | Stop site |
|---|---|---|---|---|
| | | | in reference | in reference |
| Exon | mRNA | mRNA | to SEQ | to SEQ |
| Number | start site | stop site | ID NO: 2 | ID NO: 2 |
| exon 1C | 1 | 158 | 1137 | 1294 |
| exon 2 | 159 | 646 | 7839 | 8326 |
| exon 3 | 647 | 706 | 9413 | 9472 |
| exon 4 | 707 | 802 | 12527 | 12622 |
| exon 5 | 803 | 867 | 13354 | 13418 |
| exon 6 | 868 | 940 | 14704 | 14776 |
| exon 7 | 941 | 1057 | 16396 | 16512 |
| exon 8 | 1058 | 1293 | 18207 | 18442 |
| exon 9 | 1294 | 1351 | 24296 | 24353 |
| exon 10 | 1352 | 1461 | 26337 | 26446 |
| exon 11 | 1462 | 3339 | 26581 | 28458 |

TABLE 2
Functional Segments for NM_018325.3 (SEQ ID NO: 4)

| | | | Start site in | Stop site in |
|---|---|---|---|---|
| | mRNA | mRNA | reference | reference |
| Exon | start | stop | to SEQ | to SEQ |
| Number | site | site | ID NO: 2 | ID NO: 2 |
| exon 1B | 1 | 63 | 1510 | 1572 |
| exon 2 | 64 | 551 | 7839 | 8326 |
| exon 3 | 552 | 611 | 9413 | 9472 |
| exon 4 | 612 | 707 | 12527 | 12622 |
| exon 5 | 708 | 772 | 13354 | 13418 |
| exon 6 | 773 | 845 | 14704 | 14776 |
| exon 7 | 846 | 962 | 16396 | 16512 |
| exon 8 | 963 | 1198 | 18207 | 18442 |
| exon 9 | 1199 | 1256 | 24296 | 24353 |
| exon 10 | 1257 | 1366 | 26337 | 26446 |
| exon 11 | 1367 | 3244 | 26581 | 28458 |

TABLE 3
Functional Segments for NM_145005.5 (SEQ ID NO: 6)

| | | | Start site in | Stop site in |
|---|---|---|---|---|
| | mRNA | mRNA | reference | reference |
| Exon | start | stop | to SEQ | to SEQ |
| Number | site | site | ID NO: 2 | ID NO: 2 |
| exon 1A | 1 | 80 | 1137 | 1216 |
| exon 2 | 81 | 568 | 7839 | 8326 |
| exon 3 | 569 | 628 | 9413 | 9472 |
| exon 4 | 629 | 724 | 12527 | 12622 |
| exon 5B | 725 | 1871 | 13354 | 14500 |
| (exon 5 into | | | | |
| intron 5) | | | | |

TABLE 4
Functional Segments for DB079375.1 (SEQ ID NO: 7)

| | | | Start site in | Stop site in |
|---|---|---|---|---|
| | mRNA | mRNA | reference | reference |
| Exon | start | stop | to SEQ | to SEQ |
| Number | site | site | ID NO: 2 | ID NO: 2 |
| exon 1E | 1 | 35 | 1135 | 1169 |
| exon 2 | 36 | 524 | 7839 | 8326 |
| exon 3 | 525 | 562 | 9413 | 9450 |
| (EST ends before | | | | |
| end of full exon) | | | | |

TABLE 5
Functional Segments for BU194591.1 (SEQ ID NO: 8)

| | | | Start site in | Stop site in |
|---|---|---|---|---|
| | mRNA | mRNA | reference | reference |
| Exon | start | stop | to SEQ | to SEQ |
| Number | site | site | ID NO: 2 | ID NO: 2 |
| exon 1D | 1 | 36 | 1241 | 1279 |
| exon 2 | 37 | 524 | 7839 | 8326 |
| exon 3 | 525 | 584 | 9413 | 9472 |
| exon 4 | 585 | 680 | 12527 | 12622 |
| exon 5B | 681 | 798 | 13354 | 13465 |
| (exon 5 into | | | | |
| intron 5) | | | | |

### Certain Indications

In certain embodiments, provided herein are methods of treating an individual comprising administering one or more pharmaceutical compositions described herein. In certain embodiments, the individual has a neurodegenerative disease. In certain embodiments, the individual is at risk for developing a neurodegenerative disease, including, but not limited to, ALS or FTD. In certain embodiments, the individual has been identified as having a C9ORF72 associated disease. In certain embodiments, the individual has been identified as having a C9ORF72 hexanucleotide repeat expansion associated disease. In certain embodiments, provided herein are methods for prophylactically reducing C9ORF72 expression in an individual. Certain embodiments include treating an individual in need thereof by administering to an individual a therapeutically effective amount of an antisense compound targeted to a C9ORF72 nucleic acid.

In one embodiment, administration of a therapeutically effective amount of an antisense compound targeted to a C9ORF72 nucleic acid is accompanied by monitoring of C9ORF72 levels in an individual, to determine an individual's response to administration of the antisense compound. An individual's response to administration of the antisense compound may be used by a physician to determine the amount and duration of therapeutic intervention.

In certain embodiments, administration of an antisense compound targeted to a C9ORF72 nucleic acid results in reduction of C9ORF72 expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a C9ORF72 nucleic acid results in improved motor function and respiration in an animal. In certain embodiments, administration of a C90RF72 antisense compound improves motor function and respiration by at least 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to C9ORF72 are used for the preparation of a medicament for treating a patient suffering or susceptible to a neurodegenerative disease including ALS and FTD.

### Certain Combination Therapies

In certain embodiments, one or more pharmaceutical compositions described herein are co-administered with one or more other pharmaceutical agents. In certain embodiments, such one or more other pharmaceutical agents are designed to treat the same disease, disorder, or condition as the one or more pharmaceutical compositions described herein. In certain embodiments, such one or more other pharmaceutical agents are designed to treat a different disease, disorder, or condition as the one or more pharmaceutical compositions described herein. In certain embodiments, such one or more other pharmaceutical agents are designed to treat an undesired side effect of one or more pharmaceutical compositions described herein. In certain embodiments, one or more pharmaceutical compositions described herein are co-administered with another pharmaceutical agent to treat an undesired effect of that other pharmaceutical agent. In certain embodiments, one or more pharmaceutical compositions described herein are co-administered with another pharmaceutical agent to produce a combinational effect. In certain embodiments, one or more pharmaceutical compositions described herein are co-administered with another pharmaceutical agent to produce a synergistic effect.

In certain embodiments, one or more pharmaceutical compositions described herein and one or more other pharmaceutical agents are administered at the same time. In certain embodiments, one or more pharmaceutical compositions described herein and one or more other pharmaceutical agents are administered at different times. In certain embodiments, one or more pharmaceutical compositions described herein and one or more other pharmaceutical agents are prepared together in a single formulation. In certain embodiments, one or more pharmaceutical compositions described herein and one or more other pharmaceutical agents are prepared separately.

In certain embodiments, pharmaceutical agents that may be co-administered with a pharmaceutical composition described herein include Riluzole (Rilutek), Lioresal (Lioresal), and Dexpramipexole.

In certain embodiments, pharmaceutical agents that may be co-administered with a C9ORF72 specific inhibitor described herein include, but are not limited to, an additional C9ORF72 inhibitor. In certain embodiments, the co-administered pharmaceutical agent is administered prior to administration of a pharmaceutical composition described herein. In certain embodiments, the co-administered pharmaceutical agent is administered following administration of a pharmaceutical composition described herein. In certain embodiments the co-administered pharmaceutical agent is administered at the same time as a pharmaceutical composition described herein. In certain embodiments the dose of a co-administered pharmaceutical agent is the same as the dose that would be administered if the co-administered pharmaceutical agent was administered alone. In certain embodiments the dose of a co-administered pharmaceutical agent is lower than the dose that would be administered if the co-administered pharmaceutical agent was administered alone. In certain embodiments the dose of a co-administered pharmaceutical agent is greater than the dose that would be administered if the co-administered pharmaceutical agent was administered alone.

In certain embodiments, the co-administration of a second compound enhances the effect of a first compound, such that co-administration of the compounds results in an effect that is greater than the effect of administering the first compound alone. In other embodiments, the co-administration results in effects that are additive of the effects of the compounds when administered alone. In certain embodiments, the co-administration results in effects that are supra-additive of the effects of the compounds when administered alone. In certain embodiments, the first compound is an antisense compound. In certain embodiments, the second compound is an antisense compound.

### EXAMPLES

### Non-Limiting Disclosure and Incorporation by Reference

While certain compounds, compositions, and methods described herein have been described with specificity in accordance with certain embodiments, the following examples serve only to illustrate the compounds described herein and are not intended to limit the same. Each of the references recited in the present application is incorporated herein by reference in its entirety.

### Example 1: Antisense Inhibition of Human C9ORF72 in HepG2 Cells

Antisense oligonucleotides were designed targeting a C9ORF72 nucleic acid and were tested for their effects on C9ORF72 mRNA in vitro. The antisense oligonucleotides were tested in a series of experiments that had similar culture conditions. The results for each experiment are presented in separate tables shown below. Cultured HepG2 cells at a density of 20,000 cells per well were transfected using electroporation with 7,000 nM antisense oligonucleotide. After a treatment period of approximately 24 hours, RNA was isolated from the cells and C9ORF72 mRNA levels were measured by quantitative real-time PCR. Human primer probe set RTS3750 (forward sequence TGTGACAGTTGGAATGCAGTGA, designated herein as SEQ ID NO: 15: reverse sequence GCCACTTAAAGCAATCTCTGTCTTG, designated herein as SEQ ID NO: 16: probe sequence TCGACTCTTTGCCCACCGCCA, designated herein as SEQ ID NO: 17) was used to measure mRNA levels. C9ORF72 mRNA levels were adjusted according to total RNA content, as measured by RIBOGREEN®. Results are presented as percent inhibition of C9ORF72, relative to untreated control cells.

The antisense oligonucleotides in Tables 6-10 were designed as 5-10-5 MOE gapmers. The gapmers are 20 nucleosides in length, wherein the central gap segment comprises ten 2′-deoxynucleosides and is flanked by wing segments on both the 5′ end and on the 3′ end comprising five nucleosides each. Each nucleoside in the 5′ wing segment and each nucleoside in the 3′ wing segment has a MOE modification. The internucleoside linkages throughout each gapmer are phosphorothioate linkages. All cytosine residues throughout each gapmer are 5-methylcytosines. “Start site” indicates the 5′-most nucleoside to which the antisense oligonucleotide is targeted in the human gene sequence. “Stop site” indicates the 3′-most nucleoside to which the antisense oligonucleotide is targeted human gene sequence. Each antisense oligonucleotide listed in Tables 6-9 is targeted to the either human C9ORF72 mRNA sequence, designated herein as SEQ ID NO: 1 (GENBANK Accession No. NM_001256054.1) or the human C9ORF72 genomic sequence, designated herein as SEQ ID NO: 2 (the complement of GENBANK Accession No. NT_008413.18 truncated from nucleosides 27535000 to 27565000), or both. ‘n/a’ indicates that the antisense oligonucleotide did not target that particular gene sequence. The antisense oligonucleotides of Table 10 are targeted to either SEQ ID NO: 3 (GENBANK Accession No. BQ068108.1) or SEQ ID NO: 4 (GENBANK Accession No. NM_018325.3).

As shown in Tables 6-10, below, several of the oligonucleotides targeting SEQ ID NO: 1 exhibit at least 50% inhibition, including those targeted to nucleobases 90-647, 728-1541, 1598-1863, 1935-2146, 2232-2251, 2429-2576, 2632-2743, 2788-2807, 2860-2879, 2949-2968, 3062-3081, 3132-3151, and 3250-3269 of SEQ ID NO 1. These include SEQ ID NOs: 32, 33, 34, 35, 36, 37, 38, 40, 41, 42, 43, 44, 45, 46, 47, 50, 51, 53, 55, 56, 57, 61, 62, 64, 66, 67, 72, 73, 75, 76, 81, 82, 85, 89, 90, 91, 92, 93, 94, 96, 97, 100, 102, 103, 109, 111, 112, 115, 116, 117, 118, 119, 120, 121, 122, 123, 124, 125, 126, 130, 131, 132, 133, 137, 139, 140, 141, 145, 146, 149, 150, 151, 152, 153, 154, 165, 166, 168, 169, 170, 171, 174, 179, 181, 182, 183, 185, 186, 187, 188, 190, 192, 195, 197, 199, 320, 321, 322, 323, 324, 325, 326, 327, 328, 329, 330, 331, and 332. Several of the oligonucleotides exhibit at least 70% inhibition, including those targeted to nucleobases 90-359, 430-479, 550-569, 617-647, 940-959, 1013-1033, 1446-1465, 1687-1706, 1844-1863, 1935-2007, and 2679-2698 of SEQ ID NO 1. These include SEQ ID NOs: 32, 33, 34, 35, 36, 40, 41, 42, 43, 44, 47, 66, 67, 85, 96, 103, 117, 119, 154, 165, 168, 186, 320, 321, 324, 327, 328, and 331. Several of the oligonucleotides exhibit at least 80% inhibition, including those targeted to nucleobases 90-265 and 310-329. These include SEQ ID NOs: 32, 33, 35, 40, 42, and 321. Several of the oligonucleotides exhibit at least 90% inhibition, including those targeted to nucleobases 190-209 and 310-329 of SEQ ID NO 1. These include SEQ ID NOs: 40 and 321.

As shown in Tables 6-20, below, several of the oligonucleotides targeting SEQ ID NO: 2 exhibit at least 50% inhibition, including those targeted to nucleobases 1552-1572, 2187-2238, 2728-2779, 3452-2471, 3752-3771, 5025-5044, 5656-5675, 6200-6219, 7594-7613, 7840-8328, 9415-9434, 12526-12545, 13357-13524, 13642-13661, 13790-14130, 14243-14335, 14699-14777, 15587-15606, 16395-16488, 18233-18373, 24306-24340, 24472-24491, 24565-24676, 26400-26424, 26606-26982, 27054-27265, 27351-27370, 27548-27998, 28068-28087, 28181-28270, and 28369-28388 of SEQ ID NO 2. These include SEQ ID NOs: 32, 33, 34, 35, 36, 37, 38, 40, 41, 42, 43, 44, 45, 46, 47, 50, 51, 53, 55, 56, 57, 64, 67, 72, 73, 75, 76, 81, 82, 85, 89, 90, 91, 92, 93, 94, 96, 97, 100, 102, 103, 111, 112, 115, 117, 118, 119, 121, 122, 123, 124, 125, 126, 130, 131, 132, 133, 137, 139, 140, 141, 145, 146, 149, 150, 151, 152, 153, 154, 165, 166, 168, 169, 170, 171, 174, 179, 181, 182, 183, 185, 186, 187, 188, 190, 192, 195, 197, 199, 205, 206, 208, 211, 212, 224, 226, 230, 231, 250, 251, 252, 256, 300, 301, 304, 306, 307, 310, 320, 321, 322, 323, 324, 325, 326, 327, 328, 329, 330, 331, and 332. Several of the oligonucleotides exhibit at least 70% inhibition, including those targeted to nucleobases 3452-2471, 7840-8159, 8230-8249, 12526-12545, 13642-13661, 14075-14094, 14316-14335, 14758-14777, 16395-16414, 16469, 16488, 24655-24674, 26963, 26982, 27054-27126, and 27798-27817 of SEQ ID NO 2. These include SEQ ID NOs: 32, 33, 34, 35, 36, 40, 41, 42, 43, 44, 47, 67, 85, 96, 103, 117, 119, 154, 165, 168, 186, 251, 306, 320, 321, 324, 327, 328, and 331. Several of the oligonucleotides exhibit at least 80% inhibition, including those targeted to nucleobases 7848-8023 of SEQ ID NO 2. These include SEQ ID NOs: 32, 33, 35, 40, 42, and 321. Several of the oligonucleotides exhibit at least 90% inhibition, including those targeted to nucleobases 7870-7889 and 7990-8009 of SEQ ID NO 2. These include SEQ ID NOs: 40 and 321.

TABLE 6

| Target | Target | | | | |
|---|---|---|---|---|---|
| Start | Start | | | | |
| Site at | Site at | | | | |
| SEQ ID | SEQ ID | | | % | SEQ ID |
| NO: 1 | NO: 2 | Sequence | ISIS No | inhibition | NO |
| 3 | 1139 | AGCGGGACACCGTAGGTTAC | 576883 | 0 | 30 |
| 44 | 1180 | GTGGGCGGAACTTGTCGCTG | 576807 | 1 | 31 |
| 90 | 7848 | GTCACATTATCCAAATGCTC | 576808 | 85 | 32 |
| 125 | 7883 | GGTGGGCAAAGAGTCGACAT | 576809 | 82 | 33 |
| 155 | 7913 | ATCTCTGTCTTGGCAACAGC | 576810 | 78 | 34 |
| 160 | 7918 | AAGCAATCTCTGTCTTGGCA | 576811 | 81 | 35 |
| 165 | 7923 | ACTTAAAGCAATCTCTGTCT | 576812 | 78 | 36 |
| 170 | 7928 | TTGCCACTTAAAGCAATCTC | 576813 | 67 | 37 |
| 205 | 7963 | CCCAGTAAGCAAAAGTAGCT | 576814 | 66 | 38 |
| 227 | 7985 | ACTCTAGGACCAAGAATATT | 576815 | 11 | 39 |
| 232 | 7990 | GCCTTACTCTAGGACCAAGA | 576816 | 78 | 40 |
| 240 | 7998 | CCAAATGTGCCTTACTCTAG | 576817 | 73 | 41 |
| 246 | 8004 | TGGAGCCCAAATGTGCCTTA | 576818 | 81 | 42 |
| 254 | 8012 | TCTGTCTTTGGAGCCCAAAT | 576819 | 76 | 43 |
| 275 | 8033 | CCATCACTGAGAAGTACCTG | 576820 | 79 | 44 |
| 281 | 8039 | ATTTCTCCATCACTGAGAAG | 576821 | 61 | 45 |
| 288 | 8046 | AAAAGTTATTTCTCCATCAC | 576822 | 57 | 46 |
| 295 | 8053 | TGGCAAGAAAAGTTATTTCT | 576823 | 70 | 47 |
| 302 | 8060 | GTGTGGTTGGCAAGAAAAGT | 576824 | 44 | 48 |
| 313 | 8071 | CTCCATTTAGAGTGTGGTTG | 576825 | 39 | 49 |
| 330 | 8088 | TGCATTTCGAAGGATTTCTC | 576826 | 65 | 50 |
| 338 | 8096 | CCACTCTCTGCATTTCGAAG | 576827 | 67 | 51 |
| 362 | 8120 | ACAAAAAACTTTACATCTAT | 576828 | 22 | 52 |
| 376 | 8134 | CCTTTTCAGACAAGACAAAA | 576829 | 53 | 53 |
| 401 | 8159 | AAGATTAATGAAACAATAAT | 576830 | 0 | 54 |
| 411 | 8169 | GTTTCCATCAAAGATTAATG | 576831 | 62 | 55 |
| 446 | 8204 | ATTGATAGTCCATATGTGCT | 576832 | 59 | 56 |
| 452 | 8210 | AGTATAATTGATAGTCCATA | 571818 | 57 | 57 |
| 481 | 8239 | GGAGGTAGAAACTAAGTTCT | 576833 | 45 | 58 |
| 516 | 8274 | ATGTGTTAATCTATCAACAC | 576834 | 48 | 59 |
| 545 | 8303 | TGCATCCATATTCTTCCTTT | 576835 | 43 | 60 |
| 552 | n/a | TTCCTTATGCATCCATATTC | 576836 | 64 | 61 |
| 559 | n/a | CTTGTCTTTCCTTATGCATC | 576837 | 57 | 62 |
| 566 | n/a | ACATTTTCTTGTCTTTCCTT | 576838 | 43 | 63 |
| 571 | 9415 | TCTGGACATTTTCTTGTCTT | 576839 | 61 | 64 |
| 578 | 9422 | ATAATCTTCTGGACATTTTC | 576840 | 37 | 65 |
| 617 | n/a | CTCTGACCCTGATCTTCCAT | 576841 | 79 | 66 |
| 628 | 12526 | TTGGAATAATACTCTGACCC | 576842 | 73 | 67 |
| 663 | 12561 | CAGTTCCATTACAGGAATCA | 576843 | 45 | 68 |
| 697 | 12595 | CTTCAGGAACACTGTGTGAT | 576844 | 20 | 69 |
| 705 | 12603 | ATCTATTTCTTCAGGAACAC | 576845 | 46 | 70 |
| 722 | n/a | AGTACTGTATCAGCTATATC | 576846 | 46 | 71 |
| 728 | 13357 | TCATTGAGTACTGTATCAGC | 576847 | 52 | 72 |
| 734 | 13363 | TCATCATCATTGAGTACTGT | 576848 | 67 | 73 |
| 740 | 13369 | CCAATATCATCATCATTGAG | 576849 | 47 | 74 |
| 755 | 13384 | TCATGACAGCTGTCACCAAT | 576850 | 51 | 75 |
| 761 | 13390 | AAGCCTTCATGACAGCTGTC | 576851 | 52 | 76 |
| 767 | 13396 | AGAAGAAAGCCTTCATGACA | 576852 | 23 | 77 |
| 773 | 13402 | TACTTGAGAAGAAAGCCTTC | 576853 | 24 | 78 |
| 778 | 13407 | ATTCTTACTTGAGAAGAAAG | 576854 | 12 | 79 |
| 782 | 13411 | AAAAATTCTTACTTGAGAAG | 576855 | 0 | 80 |
| 817 | 13446 | AGATGGTATCTGCTTCATCC | 576856 | 61 | 81 |
| 876 | 13505 | CAATCTAAGTAGACAGTCTG | 576857 | 57 | 82 |
| 911 | 13540 | TTAAGCAACAGTTCAAATAC | 576858 | 40 | 83 |
| 978 | 13607 | CTTTAAATAGCAAATGGAAT | 576859 | 26 | 84 |
| 1013 | 13642 | GCCATGATTTCTTGTCTGGG | 576860 | 79 | 85 |
| 1056 | 13685 | GCTTTAATGAGAAGTAAAAC | 576861 | 17 | 86 |
| 1091 | 13720 | TCTACAGTACAACTTAATAT | 576862 | 39 | 87 |
| 1126 | 13755 | ATAATTTTGTTCTACGCCTA | 576863 | 44 | 88 |
| 1161 | 13790 | CACTGCTGGATGGAAAAAGA | 576864 | 65 | 89 |
| 1196 | 13825 | TGGTTTAAGGGCACAAACTC | 576865 | 52 | 90 |
| 1231 | 13860 | TTGCCCACGGGTACACAGCA | 576866 | 63 | 91 |
| 1268 | 13897 | CAGATGAGGAAATAGGTGTA | 576867 | 62 | 92 |
| 1303 | 13932 | ACACATTAGGTACTATTACT | 576868 | 63 | 93 |
| 1372 | 14001 | TTTTTATGTTCCAGGCACTG | 576869 | 59 | 94 |
| 1407 | 14036 | AATAGGAAATGTTAGCTATG | 576870 | 30 | 95 |
| 1446 | 14075 | GGCACTCAACAAATACTGGC | 576871 | 72 | 96 |
| 1482 | 14111 | TACATGTAAAGCAACTAGTA | 576872 | 55 | 97 |
| 1539 | 14168 | TAAAATTTCATGAAAATCTG | 576873 | 0 | 98 |
| 1579 | 14208 | AAGTGAATACTTTATACTTT | 576874 | 0 | 99 |
| 1614 | 14243 | CATCATGAGCCTAAAGGAAA | 576875 | 51 | 100 |
| 1651 | 14280 | GGCTCTTAGGTTAAACACAC | 576876 | 43 | 101 |
| 1673 | 14302 | TGCTTCTGATTCAAGCCATT | 576877 | 65 | 102 |
| 1687 | 14316 | ATACAGGACTAAAGTGCTTC | 576878 | 74 | 103 |
| 1731 | 14360 | CAAATGGGATTTAAAATGAT | 576879 | 0 | 104 |
| 1766 | 14395 | TGACATGTAGAGAGATTAAG | 576880 | 26 | 105 |
| 1801 | 14430 | TTATTGAAATACCATCATTT | 576881 | 34 | 106 |
| 1836 | 14465 | TAGTCAGTATAATATCATTT | 576882 | 18 | 107 |

TABLE 7

| Target | Target | | | | |
|---|---|---|---|---|---|
| Start | Start | | | | |
| Site at | Site at | | | | |
| SEQ ID | SEQ ID | | | % | SEQ ID |
| NO: 1 | NO: 2 | Sequence | ISIS No | inhibition | NO |
| 851 | n/a | GCATTGAGAAGAAAGCCTTC | 571824 | 25 | 108 |
| 1337 | n/a | AAGACCTGATCCAGGAAGGC | 571836 | 53 | 109 |
| 861 | n/a | TGAGCTGATGGCATTGAGAA | 571981 | 41 | 110 |
| 890 | 14726 | ACAACGGAACAGCCACAGGT | 571983 | 66 | 111 |
| 1420 | 26405 | TTAGTGTCAAGGCTTTTCTG | 572007 | 60 | 112 |
| 75 | 1211 | GACGGCTGACACACCAAGCG | 576884 | 8 | 113 |
| 856 | n/a | TGATGGCATTGAGAAGAAAG | 576891 | 6 | 114 |
| 917 | 14753 | TTTACTTTCTCTGCACTGCT | 576892 | 68 | 115 |
| 922 | n/a | TCTTATTTACTTTCTCTGCA | 576893 | 63 | 116 |
| 940 | 16395 | GGCATAATGTTCTGACTATC | 576894 | 71 | 117 |
| 979 | 16434 | ATAACCTGGAGCATTTTCTC | 576895 | 65 | 118 |
| 1014 | 16469 | CCCTGACTCATATTTAAATG | 576896 | 70 | 119 |
| 1049 | n/a | CCAGTTGAATCCTTTAGCAG | 576897 | 51 | 120 |
| 1084 | 18233 | CATACATGACTTGCCGGAAA | 576898 | 66 | 121 |
| 1119 | 18268 | GACATCCACATCTATGTGTG | 576899 | 63 | 122 |
| 1154 | 18303 | TGTTCATGACAGGGTGGCAT | 576900 | 66 | 123 |
| 1163 | 18312 | TTATAAATATGTTCATGACA | 576901 | 51 | 124 |
| 1191 | 18340 | CAGCTCGGATCTCATGTATC | 576902 | 52 | 125 |
| 1205 | 18354 | CTCCAGAAGGCTGTCAGCTC | 576903 | 59 | 126 |
| 1238 | 18387 | GTATCCTGAGCCATGTCTTC | 576904 | 33 | 127 |
| 1273 | 18422 | AATCAGGAGTAAAGCTTTCG | 576905 | 48 | 128 |
| 1283 | n/a | AAAATATTCAAATCAGGAGT | 576906 | 23 | 129 |
| 1304 | 24306 | TCTCTGTGTAAGACATCTTG | 576907 | 51 | 130 |
| 1309 | 24311 | GAGTGTCTCTGTGTAAGACA | 576908 | 54 | 131 |
| 1314 | 24316 | CACTAGAGTGTCTCTGTGTA | 576909 | 50 | 132 |
| 1319 | 24321 | GCTTTCACTAGAGTGTCTCT | 576910 | 60 | 133 |
| 1330 | 24332 | GATCCAGGAAGGCTTTCACT | 576911 | 35 | 134 |
| 1373 | 26358 | AAAGTACTTCTGAGAGATAA | 576912 | 38 | 135 |
| 1385 | 26370 | AACTGTGCAAGGAAAGTACT | 576913 | 43 | 136 |
| 1415 | 26400 | GTCAAGGCTTTTCTGTGAAG | 576914 | 65 | 137 |
| 1472 | 26591 | AGAGATTTAAAGGGCTTTTT | 576915 | 46 | 138 |
| 1487 | 26606 | ATCTTCAGGTTCCGAAGAGA | 576916 | 53 | 139 |
| 1511 | 26630 | CCCTCTGCTGTTAAATCAAG | 576917 | 51 | 140 |
| 1522 | 26641 | TGTTAAGATCGCCCTCTGCT | 576918 | 64 | 141 |
| 1529 | 26648 | ATTATTATGTTAAGATCGCC | 576919 | 46 | 142 |
| 1535 | 26654 | AGAGCCATTATTATGTTAAG | 576920 | 36 | 143 |
| 1571 | 26690 | ATAAAAGAGTGTAGGCCTGG | 576921 | 46 | 144 |
| 1598 | 26717 | ACACTAGTGTAGAAAGGTCT | 576922 | 55 | 145 |
| 1606 | 26725 | GTTCTTGCACACTAGTGTAG | 576923 | 62 | 146 |
| 1628 | 26747 | TAAAAAGTCATTAGAACATC | 576924 | 10 | 147 |
| 1644 | 26763 | TATTAAGTTACACATTTAAA | 576925 | 20 | 148 |
| 1679 | 26798 | CTTTACCAGCGATCATGATT | 576926 | 57 | 149 |
| 1725 | 26844 | TTCTGGAGTATGATCCAGGG | 576927 | 64 | 150 |
| 1752 | 24472 | ACTTAACTGCAATTGCTGAG | 576928 | 66 | 151 |
| | 26871 | | | | |
| 1765 | 26884 | TGTAGTGTAACTTACTTAAC | 576929 | 60 | 152 |
| 1802 | 26921 | ATGCACCTGACATCCCCTCA | 576930 | 56 | 153 |
| 1844 | 26963 | CCCAAAAGCATAAATCTAGG | 576931 | 71 | 154 |
| 1876 | 24596 | ATATTTATTATATTGTAAAC | 576932 | 0 | 155 |
| | 26995 | | | | |
| 1883 | 24603 | AGCAATAATATTTATTATAT | 576933 | 1 | 156 |
| | 27002 | | | | |
| 1887 | 24607 | AGATAGCAATAATATTTATT | 576934 | 0 | 157 |
| | 27006 | | | | |
| 1889 | 24609 | AAAGATAGCAATAATATTTA | 576935 | 0 | 158 |
| | 27008 | | | | |
| 1892 | 24612 | TTAAAAGATAGCAATAATAT | 576936 | 3 | 159 |
| | 27011 | | | | |
| 1896 | 24616 | ATCTTTAAAAGATAGCAATA | 576937 | 14 | 160 |
| | 27015 | | | | |
| 1898 | 24618 | ATATCTTTAAAAGATAGCAA | 576938 | 15 | 161 |
| | 27017 | | | | |
| 1901 | 24621 | ATTATATCTTTAAAAGATAG | 576939 | 12 | 162 |
| | 27020 | | | | |
| 1905 | 24625 | TATTATTATATCTTTAAAAG | 576940 | 6 | 163 |
| | 27024 | | | | |
| 1918 | 27037 | CAAGTTTACATCCTATTATT | 576941 | 48 | 164 |
| 1935 | 24655 | AAAACAGTAGTTGTGGTCAA | 576942 | 77 | 165 |
| | 27054 | | | | |
| 1937 | 24657 | AAAAAACAGTAGTTGTGGTC | 576943 | 69 | 166 |
| | 27056 | | | | |
| 1953 | 27072 | TGAATCATGTATTTCAAAAA | 576944 | 17 | 167 |
| 1988 | 27107 | GCCAACTCAGATTTCACCTT | 576945 | 71 | 168 |
| 2036 | 27155 | CTACACACCAAAGAATGCCA | 576946 | 69 | 169 |
| 2071 | 27190 | AGTTTTCAGTTGATTGCAGA | 576947 | 58 | 170 |
| 2127 | 27246 | CATCCTATGTTCAAGCTCAC | 576948 | 51 | 171 |
| 2162 | 27281 | TAAACATCTGCTTGATCAAT | 576949 | 44 | 172 |
| 2197 | 27316 | AATCCACAAAGTAGGATCTA | 576950 | 42 | 173 |
| 2232 | 27351 | ATTAGACATTTCTACAGACT | 576951 | 56 | 174 |
| 2325 | 27444 | CTCAACTACATAGAATATCA | 576952 | 45 | 175 |
| 2371 | 27490 | TTGGCAACAATTACTAAAAC | 576953 | 48 | 176 |
| 2400 | 27519 | TCAAAAATAATGAAAATTAA | 576954 | 0 | 177 |
| 2409 | 27528 | CAATTTGGCTCAAAAATAAT | 576955 | 3 | 178 |
| 2429 | 27548 | GGCACAGGAGGTGCACATTT | 576956 | 60 | 179 |

TABLE 8

| Target | Target | | | | |
|---|---|---|---|---|---|
| Start | Start | | | | |
| Site at | Site at | | | | |
| SEQ ID | SEQ ID | | | % | SEQ ID |
| NO: 1 | NO: 2 | Sequence | ISIS No | inhibition | NO |
| 2451 | 27570 | TAGATTTTCTAAGGAGAAAA | 576957 | 8 | 180 |
| 2486 | 27605 | ACTGACCAGTGAAATCTGAA | 576958 | 50 | 181 |
| 2522 | 27641 | GGTAAGACTTAGCAAGAAGA | 576959 | 59 | 182 |
| 2557 | 27676 | TCTCAGAGTTGCAATGATTG | 576960 | 63 | 183 |
| 2597 | 27716 | AGATCTTATTAGTTAGTATA | 576961 | 18 | 184 |
| 2632 | 27751 | AGTACTCAAGGAACTATTTT | 576962 | 57 | 185 |
| 2679 | 27798 | GGCAAACAGCAACAACTTCA | 576963 | 71 | 186 |
| 2724 | 27843 | GCACTTCAGTAAAATTTCTC | 576964 | 69 | 187 |
| 2788 | 27907 | GGTCCAAACGCATTAAGAAA | 576965 | 58 | 188 |
| 2825 | 27944 | GAATTATATTAATCAGTTAT | 576966 | 0 | 189 |
| 2860 | 27979 | TGTGTTTGTGTAACTACAAT | 576967 | 67 | 190 |
| 2895 | 28014 | ATATTACTTCCAGAATTTTA | 576968 | 19 | 191 |
| 2949 | 28068 | GGCAGAAGGGCTCTATTACC | 576969 | 59 | 192 |
| 2992 | 28111 | CATTCGAACATGTCATTTTG | 576970 | 40 | 193 |
| 3027 | 28146 | CTGATTCATGATGGGAAAGC | 576971 | 34 | 194 |
| 3062 | 28181 | GTGGTTGTCTAAAACATCAA | 576972 | 58 | 195 |
| 3097 | 28216 | ATGACTGAGCTACAGTACAA | 576973 | 47 | 196 |
| 3132 | 28251 | GGGACACTACAAGGTAGTAT | 576974 | 56 | 197 |
| 3167 | 28286 | TTAAATAAGAATCTACCATG | 576975 | 12 | 198 |
| 3250 | 28369 | GCTTTAATAACTTATTTCAC | 576976 | 54 | 199 |
| 3282 | 28401 | AGGAGAAAAGATATATAACA | 576977 | 0 | 200 |
| 3288 | 28407 | CCATTTAGGAGAAAAGATAT | 576978 | 0 | 201 |
| n/a | 1343 | TTCACCCTCAGCGAGTACTG | 576979 | 0 | 202 |
| n/a | 1403 | AGGCTGCGGTTGTTTCCCTC | 576980 | 0 | 203 |
| n/a | 1800 | GCCAGATCCCCATCCCTTGT | 576981 | 11 | 204 |
| n/a | 2187 | TCACTTCCTTTAAGCAAGTC | 576982 | 52 | 205 |
| n/a | 2209 | AGTGATGCCCAAGTCACAAT | 576983 | 53 | 206 |
| n/a | 2214 | AGTCAAGTGATGCCCAAGTC | 576984 | 47 | 207 |
| n/a | 2219 | CCATCAGTCAAGTGATGCCC | 576985 | 60 | 208 |
| n/a | 2224 | GATTACCATCAGTCAAGTGA | 576986 | 29 | 209 |
| n/a | 2229 | CAACTGATTACCATCAGTCA | 576987 | 42 | 210 |
| n/a | 2728 | GCAGTTTCCAACTGATTCAG | 576988 | 58 | 211 |
| n/a | 2760 | CGTTCTTGTTTCAGATGTAC | 576989 | 57 | 212 |
| n/a | 2862 | GCCAAACAAAATATTTTATC | 576990 | 22 | 213 |
| n/a | 2995 | TAGGTAGGCTAACCTAGTCC | 576991 | 47 | 214 |
| n/a | 3196 | TCCCAGCCCAAAGAGAAGCA | 576992 | 41 | 215 |
| n/a | 3466 | GGATCATAGCTCTCGGTAAC | 576993 | 26 | 216 |
| n/a | 3540 | AATCATAAAGCCCTCACTTC | 576994 | 7 | 217 |
| n/a | 3595 | CTGATTGGTATTTAGAAAGG | 576995 | 3 | 218 |
| n/a | 3705 | ATGCAGACATGATTACATTA | 576996 | 48 | 219 |
| n/a | 4560 | TTCATCATTAAACTGAAAAT | 576997 | 0 | 220 |
| n/a | 4613 | CTTTTAGGTTAAAAAGGTGG | 576998 | 35 | 221 |
| n/a | 4986 | ATACAGAGCCTGGCAAAACA | 576999 | 30 | 222 |
| n/a | 5036 | TTCTATTTACAGAGCATTAG | 577000 | 29 | 223 |
| n/a | 5656 | GCCTTCACATTAATTCACCA | 577001 | 62 | 224 |
| n/a | 6051 | TGTGTTATTGCCCCTAAAAA | 577002 | 24 | 225 |
| n/a | 6200 | TGTATTCACTATACTATGCC | 577003 | 52 | 226 |
| n/a | 6276 | AAGTTATTTAAAGTATAGCA | 577004 | 0 | 227 |
| n/a | 6762 | GACATTGAAGTATCAAGACA | 577005 | 34 | 228 |
| n/a | 6965 | TGTTAAGTAATCTTAGAAAA | 577006 | 0 | 229 |
| n/a | 7594 | GGCATACATTTAGAAATTCA | 577007 | 60 | 230 |
| n/a | 8309 | ACCTTATGCATCCATATTCT | 577008 | 59 | 231 |
| n/a | 8784 | GAATTCTCTTGGGAACCATT | 577009 | 42 | 232 |
| n/a | 8834 | ATATTCAACTACAGGATTTA | 577010 | 13 | 233 |
| n/a | 8884 | ATGTGTTCTTTAGATACATC | 577011 | 42 | 234 |
| n/a | 9510 | CCTTATACAGATACATGCTG | 577012 | 37 | 235 |
| n/a | 9663 | TAGATGCAATTACTATTTTC | 577013 | 34 | 236 |
| n/a | 10742 | TGTACTTCCCAAACTTGAAC | 577014 | 24 | 237 |
| n/a | 10845 | CTGAAGCTCAACAACACCAA | 577015 | 49 | 238 |
| n/a | 11684 | GTCTATAGAATCAAACTGAA | 577016 | 38 | 239 |
| n/a | 11851 | TTGAATCAATACCTAACCTC | 577017 | 23 | 240 |
| n/a | 11991 | TGCCTCTTTTAGAAAAGATC | 577018 | 44 | 241 |
| n/a | 12042 | ATGGAATCATTGGTTTATCG | 577019 | 43 | 242 |
| n/a | 12069 | AAAGCTCACTTTTATTCTTT | 577020 | 37 | 243 |
| n/a | 12333 | | | | |
| n/a | 12170 | GGTGCCGCCACCATGCCCGG | 577021 | 0 | 244 |
| n/a | 12464 | GAGAGAAGCTGGGCAATAAA | 577022 | 2 | 245 |
| n/a | 12514 | TCTGACCCTGCACAATAAAG | 577023 | 0 | 246 |
| n/a | 13016 | ATAGTGTGTGATTCAAAACG | 577024 | 17 | 247 |
| n/a | 13348 | ACTGTATCAGCTATCTAAAA | 577025 | 22 | 248 |
| n/a | 14540 | TTATTTGTATAGGAACCTAC | 577026 | 44 | 249 |
| n/a | 14699 | TGTGAGCTGATGGCACTGTA | 577027 | 61 | 250 |
| n/a | 14758 | CCTTATTTACTTTCTCTGCA | 577028 | 71 | 251 |
| n/a | 15587 | GGAATAAGGTCACTAGTTCG | 577029 | 69 | 252 |
| n/a | 17187 | ATTTGCAACAATTTTTAAAT | 577030 | 8 | 253 |
| n/a | 21808 | ATAAACTACCAATGATATCC | 577031 | 13 | 254 |
| n/a | 24337 | TACCTGATCCAGGAAGGCTT | 577032 | 40 | 255 |
| n/a | 24565 | TTCCCGAAGCATAAATCTAG | 577033 | 53 | 256 |
| n/a | 25549 | TTGAGAAGCATGAAATTCCA | 577034 | 48 | 257 |

TABLE 9

| Target | Target | | | | |
|---|---|---|---|---|---|
| Start | Start | | | | |
| Site at | Site at | | | | |
| SEQ ID | SEQ ID | | | % | SEQ ID |
| NO: 1 | NO: 2 | Sequence | ISIS No | inhibition | NO |
| 310 | 7990 | GCCTTACTCTAGGACCAAGA | 576816 | 90 | 40 |
| 75 | 1211 | GACGGCTGACACACCAAGCG | 576884 | 0 | 113 |
| 2 | 1138 | GCGGGACACCGTAGGTTACG | 577035 | 0 | 258 |
| 10 | 1146 | CTTTCCTAGCGGGACACCGT | 577036 | 1 | 259 |
| 18 | 1154 | GCACCTCTCTTTCCTAGCGG | 577037 | 0 | 260 |
| 26 | 1162 | TGTTTGACGCACCTCTCTTT | 577038 | 0 | 261 |
| 34 | 1170 | CTTGTCGCTGTTTGACGCAC | 577039 | 0 | 262 |
| 42 | 1178 | GGGCGGAACTTGTCGCTGTT | 577040 | 0 | 263 |
| 83 | 1219 | GCAGCAGGGACGGCTGACAC | 577041 | 0 | 264 |
| 95 | 1231 | AGAAGCAACCGGGCAGCAGG | 577042 | 0 | 265 |
| 103 | 1239 | CCCAAAAGAGAAGCAACCGG | 577043 | 0 | 266 |
| 111 | 1247 | ACCCCGCCCCCAAAAGAGAA | 577044 | 1 | 267 |
| 119 | 1255 | CTTGCTAGACCCCGCCCCCA | 577045 | 0 | 268 |
| 127 | 1263 | CACCTGCTCTTGCTAGACCC | 577046 | 0 | 269 |
| 135 | 1271 | TAAACCCACACCTGCTCTTG | 577047 | 0 | 270 |
| 139 | 1275 | CTCCTAAACCCACACCTGCT | 577048 | 0 | 271 |
| n/a | 1283 | ACACACACCTCCTAAACCCA | 577049 | 0 | 272 |
| n/a | 1291 | AAACAAAAACACACACCTCC | 577050 | 5 | 273 |
| n/a | 1299 | GGTGGGAAAAACAAAAACAC | 577051 | 1 | 274 |
| n/a | 1326 | CTGTGAGAGCAAGTAGTGGG | 577052 | 3 | 275 |
| n/a | 1334 | AGCGAGTACTGTGAGAGCAA | 577053 | 0 | 276 |
| n/a | 1342 | TCACCCTCAGCGAGTACTGT | 577054 | 0 | 277 |
| n/a | 1358 | TCAGGTCTTTTCTTGTTCAC | 577055 | 0 | 278 |
| n/a | 1366 | AATCTTTATCAGGTCTTTTC | 577056 | 16 | 279 |
| n/a | 1374 | TTCTGGTTAATCTTTATCAG | 577057 | 22 | 280 |
| n/a | 1382 | TTGTTTTCTTCTGGTTAATC | 577058 | 19 | 281 |
| n/a | 1390 | TTCCCTCCTTGTTTTCTTCT | 577059 | 28 | 282 |
| n/a | 1398 | GCGGTTGTTTCCCTCCTTGT | 577060 | 17 | 283 |
| n/a | 1406 | TACAGGCTGCGGTTGTTTCC | 577061 | 28 | 284 |
| n/a | 1414 | GAGCTTGCTACAGGCTGCGG | 577062 | 23 | 285 |
| n/a | 1422 | GAGTTCCAGAGCTTGCTACA | 577063 | 14 | 286 |
| n/a | 1430 | CGACTCCTGAGTTCCAGAGC | 577064 | 0 | 287 |
| n/a | 1446 | CCCGGCCCCTAGCGCGCGAC | 577065 | 0 | 288 |
| n/a | 1454 | GCCCCGGCCCCGGCCCCTAG | 577066 | 0 | 289 |
| n/a | 1465 | ACCACGCCCCGGCCCCGGCC | 577067 | 0 | 290 |
| n/a | 1473 | CCGCCCCGACCACGCCCCGG | 577068 | 0 | 291 |
| n/a | 1481 | CCCCGGGCCCGCCCCGACCA | 577069 | 0 | 292 |
| n/a | 1495 | CGCCCCGGGCCCGCCCCCGG | 577070 | 0 | 293 |
| n/a | 1503 | CGCAGCCCCGCCCCGGGCCC | 577071 | 0 | 294 |
| n/a | 1511 | ACCGCAACCGCAGCCCCGCC | 577072 | 0 | 295 |
| n/a | 1519 | GCGCAGGCACCGCAACCGCA | 577073 | 18 | 296 |
| n/a | 1520 | GGCGCAGGCACCGCAACCGC | 577074 | 17 | 297 |
| n/a | 1536 | CGCCTCCGCCGCCGCGGGCG | 577075 | 32 | 298 |
| n/a | 1544 | ACCGCCTGCGCCTCCGCCGC | 577076 | 43 | 299 |
| n/a | 1552 | CACTCGCCACCGCCTGCGCC | 577077 | 52 | 300 |
| n/a | 1553 | CCACTCGCCACCGCCTGCGC | 577078 | 52 | 301 |
| n/a | 1853 | GGTCCCCGGGAAGGAGACAG | 577079 | 41 | 302 |
| n/a | 2453 | AACAACTGGTGCATGGCAAC | 577080 | 42 | 303 |
| n/a | 2753 | GTTTCAGATGTACTATCAGC | 577081 | 63 | 304 |
| n/a | 3053 | AAGGTGAAGTTCATATCACT | 577082 | 10 | 305 |
| n/a | 3452 | GGTAACTTCAAACTCTTGGG | 577083 | 70 | 306 |
| n/a | 3752 | GGTTCATGAGAGGTTTCCCA | 577084 | 53 | 307 |
| n/a | 4052 | TACTGAATTGCTTAGTTTTA | 577085 | 25 | 308 |
| n/a | 4425 | CTAACAGAATAAGAAAAAAA | 577086 | 0 | 309 |
| n/a | 5025 | GAGCATTAGATGAGTGCTTT | 577087 | 52 | 310 |
| n/a | 5325 | TGCATTCCTAAGCAATGTGT | 577088 | 28 | 311 |
| n/a | 5661 | TCTAGGCCTTCACATTAATT | 577089 | 37 | 312 |
| n/a | 5961 | CCTGTCTATGCCTAGGTGAA | 577090 | 19 | 313 |
| n/a | 6261 | TAGCACATACAATTATTACA | 577091 | 38 | 314 |
| n/a | 6566 | GAGGAGAAGAACATAAACGC | 577092 | 20 | 315 |
| n/a | 6866 | TACCACAAGTCTGGAGCCAT | 577093 | 27 | 316 |
| n/a | 7166 | GATACTGGATTGTTGAAACT | 577094 | 1 | 317 |
| n/a | 7466 | TAGTATGACTGGAGATTTGG | 577095 | 1 | 318 |
| n/a | 7766 | ATCAAAACCCCAAATGATTT | 577096 | 13 | 319 |
| 160 | 7840 | ATCCAAATGCTCCGGAGATA | 577097 | 78 | 320 |
| 190 | 7870 | TCGACATCACTGCATTCCAA | 577098 | 95 | 321 |
| 220 | 7900 | CAACAGCTGGAGATGGCGGT | 577099 | 56 | 322 |
| 250 | 7930 | ATTTGCCACTTAAAGCAATC | 577100 | 62 | 323 |
| 340 | 8020 | GTACCTGTTCTGTCTTTGGA | 577101 | 76 | 324 |
| 370 | 8050 | CAAGAAAAGTTATTTCTCCA | 577102 | 65 | 325 |
| 400 | 8080 | GAAGGATTTCTCCATTTAGA | 577103 | 50 | 326 |
| 430 | 8110 | TTACATCTATAGCACCACTC | 577104 | 73 | 327 |
| 460 | 8140 | TCACTCCCTTTTCAGACAAG | 577105 | 73 | 328 |
| 490 | 8170 | AGTTTCCATCAAAGATTAAT | 577106 | 55 | 329 |
| 520 | 8200 | ATAGTCCATATGTGCTGCGA | 577107 | 57 | 330 |
| 550 | 8230 | AACTAAGTTCTGTCTGTGGA | 577108 | 71 | 331 |
| 580 | 8260 | CAACACACACTCTATGAAGT | 577109 | 54 | 332 |
| 610 | 8290 | TTCCTTTCCGGATTATATGT | 577110 | 0 | 333 |

TABLE 10

| Target | Target | | | | |
|---|---|---|---|---|---|
| SEQ ID | Start | | | % | SEQ ID |
| NO | Site | ISIS No | Sequence | inhibition | NO |
| 3 | 751 | 576885 | TTTCCATTACAGGAATCACT | 63 | 334 |
| 3 | 807 | 576886 | ATCAGCCTATATCTATTTCC | 15 | 335 |
| 3 | 855 | 576887 | TCAATGACCAGGCGGTCCCC | 0 | 336 |
| 3 | 905 | 576888 | CTTTTTATGGAAAAGGAAAA | 0 | 337 |
| 3 | 984 | 576889 | TGTTTCCCCAAAAATTTCTG | 0 | 338 |
| 4 | 50 | 576890 | AGATATCCACTCGCCACCGC | 42 | 339 |

### Example 2: Dose-Dependent Antisense Inhibition of Human C9ORF72 in HepG2 Cells

Antisense oligonucleotides from the study described above exhibiting significant in vitro inhibition of C9ORF72 mRNA were selected and tested at various doses in HepG2 cells. The antisense oligonucleotides were tested in a series of experiments that had similar culture conditions. The results for each experiment are presented in separate tables shown below. Cells were plated at a density of 20,000 cells per well and transfected using electroporation with 82.3 nM, 246.9 nM, 740.7 nM, 2,222.2 nM, 6,666.7 nM, or 20,000 nM concentrations of antisense oligonucleotide. After a treatment period of approximately 16 hours, RNA was isolated from the cells and C9ORF72 mRNA levels were measured by quantitative real-time PCR. Human C9ORF72 primer probe set RTS3750 was used to measure mRNA levels. C9ORF72 mRNA levels were adjusted according to total RNA content, as measured by RIBOGREEN®. Results are presented as percent inhibition of C9ORF72, relative to untreated control cells.

The half maximal inhibitory concentration (ICso) of each oligonucleotide is also presented in Tables 11-13. As illustrated, C90RF72 mRNA levels were reduced in a dose-dependent manner in the antisense oligonucleotide treated cells.

TABLE 11

| | 82.3 | 246.9 | 740.7 | 2222.2 | 6666.7 | 20000.0 | IC₅₀ |
|---|---|---|---|---|---|---|---|
| ISIS No | nM | nM | nM | nM | nM | nM | ₍μM) |
| 576816 | 5 | 23 | 49 | 76 | 91 | 96 | 0.9 |
| 576817 | 8 | 2 | 6 | 29 | 58 | 83 | 4.7 |
| 576818 | 0 | 22 | 31 | 68 | 87 | 90 | 1.4 |
| 576819 | 0 | 12 | 44 | 72 | 81 | 86 | 1.4 |
| 576820 | 18 | 24 | 52 | 78 | 91 | 93 | 0.7 |
| 576841 | 23 | 19 | 29 | 52 | 75 | 85 | 1.6 |
| 576842 | 6 | 12 | 13 | 37 | 53 | 83 | 4.1 |
| 576860 | 9 | 24 | 54 | 70 | 83 | 87 | 1.0 |
| 576878 | 1 | 9 | 26 | 61 | 77 | 83 | 2.0 |
| 576931 | 16 | 21 | 24 | 49 | 77 | 83 | 1.8 |
| 576942 | 6 | 16 | 26 | 57 | 78 | 85 | 1.8 |

TABLE 12

| | 82.3 | 246.9 | 740.7 | 2222.2 | 6666.7 | 20000.0 | IC₅₀ |
|---|---|---|---|---|---|---|---|
| ISIS No | nM | nM | nM | nM | nM | nM | ₍μM) |
| 576894 | 9 | 30 | 38 | 61 | 75 | 84 | 1.3 |
| 576896 | 17 | 17 | 28 | 47 | 66 | 76 | 2.5 |
| 576927 | 3 | 26 | 40 | 60 | 79 | 81 | 1.5 |
| 576943 | 37 | 37 | 55 | 77 | 84 | 82 | 0.4 |
| 576945 | 20 | 41 | 56 | 73 | 83 | 84 | 0.6 |
| 576946 | 8 | 28 | 46 | 69 | 81 | 88 | 1.0 |
| 576963 | 0 | 0 | 25 | 51 | 63 | 83 | 2.9 |
| 576964 | 11 | 18 | 37 | 58 | 73 | 77 | 1.8 |
| 576967 | 19 | 31 | 48 | 68 | 77 | 85 | 0.9 |
| 577028 | 6 | 19 | 25 | 59 | 79 | 88 | 1.6 |
| 577029 | 7 | 22 | 44 | 67 | 77 | 85 | 1.3 |

TABLE 13

| | 82.3 | 246.9 | 740.7 | 2222.2 | 6666.7 | 20000.0 | IC₅₀ |
|---|---|---|---|---|---|---|---|
| ISIS No | nM | nM | nM | nM | nM | nM | ₍μM) |
| 576960 | 0 | 12 | 28 | 49 | 58 | 78 | 3.2 |
| 576974 | 25 | 45 | 65 | 70 | 65 | 78 | 0.5 |
| 576816 | 18 | 36 | 53 | 82 | 91 | 95 | 0.6 |
| 577097 | 22 | 20 | 31 | 63 | 82 | 94 | 1.1 |
| 577101 | 16 | 23 | 39 | 62 | 80 | 89 | 1.2 |
| 577105 | 0 | 4 | 30 | 48 | 78 | 92 | 2.0 |
| 577104 | 4 | 1 | 16 | 56 | 80 | 92 | 2.0 |
| 577108 | 0 | 0 | 24 | 52 | 76 | 83 | 2.9 |
| 577083 | 0 | 0 | 24 | 50 | 73 | 74 | 3.0 |
| 577078 | 0 | 0 | 10 | 15 | 30 | 75 | 10.8 |
| 577077 | 0 | 0 | 22 | 22 | 51 | 83 | 5.0 |

### Example 3: Dose-Dependent Antisense Inhibition of Human C9ORF72 in HepG2 Cells

Antisense oligonucleotides from the study described above exhibiting significant in vitro inhibition of C9ORF72 mRNA were selected and tested at various doses in HepG2 cells. The antisense oligonucleotides were tested in a series of experiments that had similar culture conditions. The results for each experiment are presented in separate tables shown below. Cells were plated at a density of 20,000 cells per well and transfected using electroporation with 246.9 nM, 740.7 nM, 2,222.2 nM, 6,666.7 nM, or 20,000 nM concentrations of antisense oligonucleotide. After a treatment period of approximately 16 hours, RNA was isolated from the cells and C9ORF72 total mRNA levels, as well as mRNA levels of the exon 1 transcript, were measured by quantitative real-time PCR. Human C9ORF72 primer probe set RTS3750 was used to measure total C9ORF72 mRNA levels. Primer probe set RTS3905 (forward sequence GGGTCTAGCAAGAGCAGGTG, designated herein as SEQ ID NO: 18; reverse sequence GTCTTGGCAACAGCTGGAGAT, designated herein as SEQ ID NO: 19; probe sequence TGATGTCGACTCTTTGCCCACCGC, designated herein as SEQ ID NO: 20) was used to measure exon 1 message transcript. C9ORF72 mRNA levels were adjusted according to total RNA content, as measured by RIBOGREEN®. Results are presented as percent inhibition of C9ORF72, relative to untreated control cells.

The half maximal inhibitory concentration (IC₅₀) of each oligonucleotide is also presented in Tables 14 and 15. As illustrated, C9ORF72 mRNA levels were reduced in a dose-dependent manner in the antisense oligonucleotide treated cells. ‘n.d.’ indicates that there is no data for that particular dose.

TABLE 14
% inhibition of total C9ORF72 mRNA levels

| | 246.9 | 740.7 | 2222.2 | 6666.7 | 20000.0 | IC₅₀ |
|---|---|---|---|---|---|---|
| ISIS No | nM | nM | nM | nM | nM | ₍μM) |
| 576816 | 29 | 53 | 84 | 90 | 92 | 0.60 |
| 576820 | 20 | 42 | 70 | 87 | 75 | 1.19 |
| 576860 | 25 | 53 | 72 | 86 | 85 | 0.80 |
| 576974 | 36 | 49 | 64 | 65 | 68 | 0.95 |
| 577041 | 3 | 0 | 0 | 0 | 0 | >20.00 |
| 577042 | 0 | 2 | 0 | 3 | 0 | >20.00 |
| 577061 | 0 | 3 | 0 | 4 | 0 | >20.00 |
| 577065 | 7 | 0 | 1 | 6 | 0 | >20.00 |
| 577069 | 3 | 0 | 3 | 0 | 0 | >20.00 |
| 577073 | 7 | 0 | 8 | 11 | 0 | >20.00 |
| 577074 | 0 | 7 | 11 | 15 | 0 | >20.00 |
| 577078 | 0 | 2 | 20 | 65 | 81 | 5.22 |
| 577083 | 0 | 19 | 55 | 71 | 75 | 3.35 |
| 577088 | 6 | 11 | 49 | 61 | 74 | 3.93 |
| 577097 | 3 | 38 | 62 | 78 | 82 | 1.94 |

TABLE 15
% inhibition of C9ORF72 exon 1 mRNA levels

| | 246.9 | 740.7 | 2222.2 | 6666.7 | 20000.0 | IC₅₀ |
|---|---|---|---|---|---|---|
| ISIS No | nM | nM | nM | nM | nM | ₍μM) |
| 576794 | 42 | 67 | n.d. | 93 | 87 | 0.27 |
| 576816 | 45 | 78 | 93 | n.d. | n.d. | 0.26 |
| 576820 | 54 | 65 | 92 | 98 | 94 | <0.247 |
| 576860 | 43 | 36 | 71 | 95 | 91 | 0.66 |
| 577041 | 0 | 0 | 49 | 4 | 31 | >20.00 |
| 577042 | 9 | 15 | 0 | 33 | 12 | >20.00 |
| 577061 | 8 | 36 | 70 | 67 | 76 | 2.03 |
| 577065 | 20 | 55 | 67 | 82 | 62 | 1.06 |
| 577069 | 22 | 24 | 61 | 74 | 70 | 2.16 |
| 577073 | 4 | 62 | 69 | 82 | 81 | 1.21 |
| 577074 | 8 | 49 | 69 | 85 | 85 | 1.29 |
| 577078 | 0 | 21 | 59 | 81 | n.d. | 1.90 |
| 577083 | 30 | 43 | 85 | 88 | 92 | 0.71 |
| 577088 | 38 | 44 | 79 | 87 | 91 | 0.61 |
| 577097 | 17 | 47 | 52 | 94 | 89 | 1.27 |

### Example 4: Antisense Inhibition of Human C9ORF72 in HepG2 Cells

Antisense oligonucleotides were designed targeting the hexanucleotide repeat expansion of a C9ORF72 nucleic acid and were tested for their effects on C9ORF72 mRNA in vitro. The antisense oligonucleotides were tested in a series of experiments that had similar culture conditions. The results for each experiment are presented in separate tables shown below. ISIS 576816 and ISIS 577065 were included in these assays for comparison. Cultured C9ORF72 fibroblasts at a density of 35,000 cells per well were transfected using electroporation with 7,000 nM antisense oligonucleotide. After a treatment period of approximately 24 hours, RNA was isolated from the cells and C9ORF72 mRNA levels were measured by quantitative real-time PCR. Human primer probe sets RTS3750, RTS 3905, or RTS4097 (forward sequence CAAGCCACCGTCTCACTCAA, designated herein as SEQ ID NO: 24: reverse sequence GTAGTGCTGTCTACTCCAGAGAGTTACC, designated herein as SEQ ID NO: 25; probe sequence CTTGGCTTCCCTCAAAAGACTGGCTAATGT, designated herein as SEQ ID NO: 26) were used to measure mRNA levels. RTS3750 targets exon 2 of the mRNA transcripts and, therefore, measures total mRNA transcripts. RTS3905 targets the hexanucleotide repeat expansion containing transcript and, therefore, measures only mRNA transcripts that contain the hexanucleotide repeat expansion. RTS4097 targets the gene sequence at a site 3′ of the hexanucleotide repeat expansion. mRNA levels were adjusted according to total RNA content, as measured by RIBOGREEN®. Results are presented as percent inhibition of C9ORF72, relative to untreated control cells. ‘n.d.’ indicates that there is no data for that particular antisense oligonucleotide.

The antisense oligonucleotides in Table 16 were designed as uniform MOE oligonucleotides, or 3-10-3 MOE, 4-10-3 MOE, 4-10-4 MOE, 5-10-4 MOE, or 5-10-5 MOE gapmers. The uniform MOE oligonucleotides are 20 nucleosides in length, wherein each nucleoside comprises a 2′-MOE group. The 3-10-3 MOE gapmers are 16 nucleosides in length, wherein the central gap segment comprises ten 2′-deoxynucleosides and is flanked by wing segments on both the 5′ end and on the 3′ end comprising three nucleosides each. The 4-10-3 gapmers are 17 nucleosides in length, wherein the central gap segment comprises ten 2′-deoxynucleosides and is flanked by wing segments on both the 5′ end and on the 3′ end comprising four and three nucleosides, respectively. The 4-10-4 gapmers are 18 nucleosides in length, wherein the central gap segment comprises ten 2′-deoxynucleosides and is flanked by wing segments on both the 5′ end and on the 3′ end comprising four nucleosides each. The 5-10-4 gapmers are 19 nucleosides in length, wherein the central gap segment comprises ten 2′-deoxynucleosides and is flanked by wing segments on both the 5′ end and on the 3′ end comprising five and four nucleosides, respectively. The 5-10-5 gapmers are 20 nucleosides in length, wherein the central gap segment comprises ten 2′-deoxynucleosides and is flanked by wing segments on both the 5′ end and on the 3′ end comprising five nucleosides each. Each nucleoside in the 5′ wing segment and each nucleoside in the 3′ wing segment comprises a 2′-MOE group. The internucleoside linkages throughout each oligonucleotide are phosphorothioate linkages. All cytosine residues throughout each oligonucleotide are 5-methylcytosines. “Start site” indicates the 5′-most nucleoside to which the antisense oligonucleotide is targeted in the human gene sequence. “Stop site” indicates the 3′-most nucleoside to which the antisense oligonucleotide is targeted human gene sequence. Each antisense oligonucleotide listed in Table 16 is targeted to the human C9ORF72 genomic sequence, designated herein as SEQ ID NO: 2 (the complement of GENBANK Accession No. NT_008413.18 truncated from nucleosides 27535000 to 27565000) or SEQ ID NO: 13, which is an expanded version of the hexanucleotide repeat from intron 1 of the C9ORF72 gene.

The data indicates that certain antisense oligonucleotides preferentially inhibit levels of C9ORF72 mRNA transcript levels that contain the hexanucleotide repeat.

TABLE 16

| Target | Target | | | | | | | |
|---|---|---|---|---|---|---|---|---|
| Start | Start | | | | | | | |
| Site on | Site on | | | | % inhi- | % inhi- | % inhi- | SEQ |
| SEQ ID | SEQ ID | | | ISIS | bition | bition | bition | ID |
| NO: 2 | NO: 13 | Motif | Sequence | NO | (RTS3750) | (RTS3905) | (RTS4097) | NO |
| 1457 | 1 | Uniform | CCGGCCCCGGCCC | 573674 | 0 | 34 | 0 | 340 |
| | 7 | MOE | CGGCCCC | | | | | |
| | 13 | | | | | | | |
| 1458 | 2 | Uniform | CCCGGCCCCGGCC | 573675 | 0 | 28 | 0 | 341 |
| | 8 | MOE | CCGGCCC | | | | | |
| | 14 | | | | | | | |
| 1459 | 3 | Uniform | CCCCGGCCCCGGC | 573676 | 0 | 34 | 0 | 342 |
| | 9 | MOE | CCCGGCC | | | | | |
| | 15 | | | | | | | |
| 1460 | 4 | Uniform | GCCCCGGCCCCGG | 573677 | 4 | 41 | 0 | 343 |
| | 10 | MOE | CCCCGGC | | | | | |
| | 16 | | | | | | | |
| n/a | 5 | Uniform | GGCCCCGGCCCCG | 573678 | 12 | 11 | 6 | 344 |
| | 11 | MOE | GCCCCGG | | | | | |
| | 17 | | | | | | | |
| n/a | 6 | Uniform | CGGCCCCGGCCCC | 573679 | 0 | 0 | 0 | 345 |
| | 12 | MOE | GGCCCCG | | | | | |
| 1457 | 1 | Uniform | CGGCCCCGGCCCC | 573680 | 10 | 6 | 0 | 346 |
| | 7 | MOE | GGCCCC | | | | | |
| | 13 | | | | | | | |
| 1458 | 2 | Uniform | CCGGCCCCGGCCC | 573681 | 13 | 23 | 0 | 347 |
| | 8 | MOE | CGGCCC | | | | | |
| | 14 | | | | | | | |
| 1459 | 3 | Uniform | CCCGGCCCCGGCC | 573682 | 2 | 48 | 0 | 348 |
| | 9 | MOE | CCGGCC | | | | | |
| | 15 | | | | | | | |
| 1460 | 4 | Uniform | CCCCGGCCCCGGC | 573683 | 0 | 38 | 0 | 349 |
| | 10 | MOE | CCCGGC | | | | | |
| | 16 | | | | | | | |
| 1461 | 5 | Uniform | GCCCCGGCCCCGG | 573684 | 0 | 0 | 0 | 350 |
| | 11 | MOE | CCCCGG | | | | | |
| | 17 | | | | | | | |
| n/a | 6 | Uniform | GGCCCCGGCCCCG | 573685 | 0 | 27 | 0 | 351 |
| | 12 | MOE | GCCCCG | | | | | |
| | 18 | | | | | | | |
| 1457 | 1 | Uniform | GGCCCCGGCCCCG | 573686 | 0 | 40 | 0 | 352 |
| | 7 | MOE | GCCCC | | | | | |
| | 13 | | | | | | | |
| | 19 | | | | | | | |
| 1458 | 2 | Uniform | CGGCCCCGGCCCC | 573687 | 0 | 0 | 0 | 353 |
| | 8 | MOE | GGCCC | | | | | |
| | 14 | | | | | | | |
| 1459 | 3 | Uniform | CCGGCCCCGGCCC | 573688 | 22 | 0 | 0 | 354 |
| | 9 | MOE | CGGCC | | | | | |
| | 15 | | | | | | | |
| 1460 | 4 | Uniform | CCCGGCCCCGGCC | 573689 | 0 | 22 | 0 | 355 |
| | 10 | MOE | CCGGC | | | | | |
| | 16 | | | | | | | |
| 1461 | 5 | Uniform | CCCCGGCCCCGGC | 573690 | 15 | 43 | 0 | 356 |
| | 11 | MOE | CCCGG | | | | | |
| | 17 | | | | | | | |
| 1462 | 6 | Uniform | GCCCCGGCCCCGG | 573691 | 10 | 16 | 0 | 357 |
| | 12 | MOE | CCCCG | | | | | |
| | 18 | | | | | | | |
| 1457 | 1 | Uniform | GCCCCGGCCCCGG | 573692 | 6 | 65 | 0 | 358 |
| | 7 | MOE | CCCC | | | | | |
| 1463 | 13 | | | | | | | |
| | 19 | | | | | | | |
| 1458 | 2 | Uniform | GGCCCCGGCCCCG | 573693 | 9 | 0 | 0 | 359 |
| | 8 | MOE | GCCC | | | | | |
| | 14 | | | | | | | |
| | 20 | | | | | | | |
| 1459 | 3 | Uniform | CGGCCCCGGCCCC | 573694 | 10 | 0 | 0 | 360 |
| | 9 | MOE | GGCC | | | | | |
| | 15 | | | | | | | |
| 1460 | 4 | Uniform | CCGGCCCCGGCCC | 573695 | 3 | 42 | 0 | 361 |
| | 10 | MOE | CGGC | | | | | |
| | 16 | | | | | | | |
| 1461 | 5 | Uniform | CCCGGCCCCGGCC | 573696 | 0 | 23 | 0 | 362 |
| | 11 | MOE | CCGG | | | | | |
| | 17 | | | | | | | |
| 1462 | 6 | Uniform | CCCCGGCCCCGGC | 573697 | 0 | 28 | 0 | 363 |
| | 12 | MOE | CCCG | | | | | |
| | 18 | | | | | | | |
| 1457 | 1 | Uniform | CCCCGGCCCCGGC | 573698 | 1 | 68 | 0 | 364 |
| | 7 | MOE | CCC | | | | | |
| 1463 | 13 | | | | | | | |
| | 19 | | | | | | | |
| 1458 | 2 | Uniform | GCCCCGGCCCCGG | 573699 | 0 | 31 | 0 | 365 |
| | 8 | MOE | CCC | | | | | |
| 1464 | 14 | | | | | | | |
| | 20 | | | | | | | |
| 1459 | 3 | Uniform | GGCCCCGGCCCCG | 573700 | 7 | 2 | 2 | 366 |
| | 9 | MOE | GCC | | | | | |
| | 15 | | | | | | | |
| | 21 | | | | | | | |
| 1460 | 4 | Uniform | CGGCCCCGGCCCC | 573701 | 15 | 1 | 8 | 367 |
| | 10 | MOE | GGC | | | | | |
| | 16 | | | | | | | |
| 1461 | 5 | Uniform | CCGGCCCCGGCCC | 573702 | 26 | 0 | 0 | 368 |
| | 11 | MOE | CGG | | | | | |
| | 17 | | | | | | | |
| 1462 | 6 | Uniform | CCCGGCCCCGGCC | 573703 | 12 | 52 | 10 | 369 |
| | 12 | MOE | CCG | | | | | |
| | 18 | | | | | | | |
| 1457 | 1 | 5-10-5 | CCGGCCCCGGCCC | 573716 | 0 | 93 | 46 | 340 |
| | 7 | MOE | CGGCCCC | | | | | |
| | 13 | | | | | | | |
| 1458 | 2 | 5-10-5 | CCCGGCCCCGGCC | 573717 | 0 | 98 | 0 | 341 |
| | 8 | MOE | CCGGCCC | | | | | |
| | 14 | | | | | | | |
| 1459 | 3 | 5-10-5 | CCCCGGCCCCGGC | 573718 | 0 | 98 | 2 | 342 |
| | 9 | MOE | CCCGGCC | | | | | |
| | 15 | | | | | | | |
| 1460 | 4 | 5-10-5 | GCCCCGGCCCCGG | 573719 | 0 | 68 | 19 | 343 |
| | 10 | MOE | CCCCGGC | | | | | |
| | 16 | | | | | | | |
| n/a | 5 | 5-10-5 | GGCCCCGGCCCCG | 573720 | 13 | 90 | 18 | 344 |
| | 11 | MOE | GCCCCGG | | | | | |
| | 17 | | | | | | | |
| n/a | 6 | 5-10-5 | CGGCCCCGGCCCC | 573721 | 0 | 98 | 18 | 345 |
| | 12 | MOE | GGCCCCG | | | | | |
| 1457 | 1 | 5-10-4 | CGGCCCCGGCCCC | 573722 | 0 | 97 | 0 | 346 |
| | 7 | MOE | GGCCCC | | | | | |
| | 13 | | | | | | | |
| 1458 | 2 | 5-10-4 | CCGGCCCCGGCCC | 573723 | 0 | n.d. | 8 | 347 |
| | 8 | MOE | CGGCCC | | | | | |
| | 14 | | | | | | | |
| 1459 | 3 | 5-10-4 | CCCGGCCCCGGCC | 573724 | 0 | 94 | 28 | 348 |
| | 9 | MOE | CCGGCC | | | | | |
| | 15 | | | | | | | |
| 1460 | 4 | 5-10-4 | CCCCGGCCCCGGC | 573725 | 0 | 94 | 7 | 349 |
| | 10 | MOE | CCCGGC | | | | | |
| | 16 | | | | | | | |
| 1461 | 5 | 5-10-4 | GCCCCGGCCCCGG | 573726 | 0 | n.d. | 28 | 350 |
| | 11 | MOE | CCCCGG | | | | | |
| | 17 | | | | | | | |
| n/a | 6 | 5-10-4 | GGCCCCGGCCCCG | 573727 | 0 | 98 | 40 | 351 |
| | 12 | MOE | GCCCCG | | | | | |
| | 18 | | | | | | | |
| 1457 | 1 | 4-10-4 | GGCCCCGGCCCCG | 573728 | 0 | 97 | 19 | 352 |
| | 7 | MOE | GCCCC | | | | | |
| | 13 | | | | | | | |
| | 19 | | | | | | | |
| 1458 | 2 | 4-10-4 | CGGCCCCGGCCCC | 573729 | 0 | n.d. | 36 | 353 |
| | 8 | MOE | GGCCC | | | | | |
| | 14 | | | | | | | |
| 1459 | 3 | 4-10-4 | CCGGCCCCGGCCC | 573730 | 0 | 94 | 24 | 354 |
| | 9 | MOE | CGGCC | | | | | |
| | 15 | | | | | | | |
| 1460 | 4 | 4-10-4 | CCCGGCCCCGGCC | 573731 | 0 | 97 | 13 | 355 |
| | 10 | MOE | CCGGC | | | | | |
| | 16 | | | | | | | |
| 1461 | 5 | 4-10-4 | CCCCGGCCCCGGC | 573732 | 0 | 97 | 1 | 356 |
| | 11 | MOE | CCCGG | | | | | |
| | 17 | | | | | | | |
| 1462 | 6 | 4-10-4 | GCCCCGGCCCCGG | 573733 | 0 | n.d. | 0 | 357 |
| | 12 | MOE | CCCCG | | | | | |
| | 18 | | | | | | | |
| 1457 | 1 | 4-10-3 | GCCCCGGCCCCGG | 573734 | 0 | 96 | 0 | 358 |
| | 7 | MOE | CCCC | | | | | |
| 1463 | 13 | | | | | | | |
| | 19 | | | | | | | |
| 1458 | 2 | 4-10-3 | GGCCCCGGCCCCG | 573735 | 0 | 94 | 21 | 359 |
| | 8 | MOE | GCCC | | | | | |
| | 14 | | | | | | | |
| | 20 | | | | | | | |
| 1459 | 3 | 4-10-3 | CGGCCCCGGCCCC | 573736 | 0 | 93 | 43 | 360 |
| | 9 | MOE | GGCC | | | | | |
| | 15 | | | | | | | |
| 1460 | 4 | 4-10-3 | CCGGCCCCGGCCC | 573737 | 0 | 96 | 19 | 361 |
| | 10 | MOE | CGGC | | | | | |
| | 16 | | | | | | | |
| 1461 | 5 | 4-10-3 | CCCGGCCCCGGCC | 573738 | 0 | n.d. | 24 | 362 |
| | 11 | MOE | CCGG | | | | | |
| | 17 | | | | | | | |
| 1462 | 6 | 4-10-3 | CCCCGGCCCCGGC | 573739 | 0 | n.d. | 34 | 363 |
| | 12 | MOE | CCCG | | | | | |
| | 18 | | | | | | | |
| 1457 | 1 | 3-10-3 | CCCCGGCCCCGGC | 573740 | 0 | n.d. | 4 | 364 |
| | 7 | MOE | CCC | | | | | |
| 1463 | 13 | | | | | | | |
| | 19 | | | | | | | |
| 1458 | 2 | 3-10-3 | GCCCCGGCCCCGG | 573741 | 0 | 95 | 6 | 365 |
| | 8 | MOE | CCC | | | | | |
| 1464 | 14 | | | | | | | |
| | 20 | | | | | | | |
| 1459 | 3 | 3-10-3 | GGCCCCGGCCCCG | 573742 | 23 | 97 | 49 | 366 |
| | 9 | MOE | GCC | | | | | |
| | 15 | | | | | | | |
| | 21 | | | | | | | |
| 1460 | 4 | 3-10-3 | CGGCCCCGGCCCC | 573743 | 0 | 96 | 0 | 367 |
| | 10 | MOE | GGC | | | | | |
| | 16 | | | | | | | |
| 1461 | 5 | 3-10-3 | CCGGCCCCGGCCC | 573744 | 0 | 94 | 34 | 368 |
| | 11 | MOE | CGG | | | | | |
| | 17 | | | | | | | |
| 1462 | 6 | 3-10-3 | CCCGGCCCCGGCC | 573745 | 0 | 94 | 34 | 368 |
| | 12 | MOE | CCG | | | | | |
| | 18 | | | | | | | |
| 7990 | n/a | 5-10-5 | GCCTTACTCTAGG | 576816 | 83 | 91 | 29 | 40 |
| | | MOE | ACCAAGA | | | | | |
| 1446 | n/a | 5-10-5 | CCCGGCCCCTAGC | 577065 | 0 | 87 | 34 | 288 |
| | | MOE | GCGCGAC | | | | | |

### Example 5: In Vivo Rodent Inhibition and Tolerability with Treatment of C9ORF72 Antisense Oligonucleotides

In order to assess the tolerability of inhibition of C9ORF72 expression in vivo, antisense oligonucleotides targeting a murine C9ORF72 nucleic acid were designed and assessed in mouse and rat models.

ISIS 571883 was designed as a 5-10-5 MOE gapmer, 20 nucleosides in length, wherein the central gap segment comprises ten 2′-deoxynucleosides and is flanked by wing segments on both the 5′ end and on the 3′ end comprising five nucleosides each. Each nucleoside in the 5′ wing segment and each nucleoside in the 3′ wing segment has a MOE modification. The internucleoside linkages are phosphorothioate linkages. All cytosine residues throughout the gapmer are 5-methylcytosines. ISIS 571883 has a target start site of nucleoside 33704 on the murine C9ORF72 genomic sequence, designated herein as SEQ ID NO: 11 (the complement of GENBANK Accession No. NT_166289.1 truncated from nucleosides 3587000 to 3625000).

ISIS 603538 was designed as a 5-10-5 MOE gapmer, 20 nucleosides in length, wherein the central gap segment comprises ten 2′-deoxynucleosides and is flanked by wing segments on both the 5′ end and on the 3′ end comprising five nucleosides each. Each nucleoside in the 5′ wing segment and each nucleoside in the 3′ wing segment has a MOE modification. The internucleoside linkages are either phosphorothioate linkages or phosphate ester linkages (Gs Ao Co Co Gs Cs Ts Ts Gs As Gs Ts Ts Ts Gs Co Co Ao Cs A: wherein ‘s’ denotes a phosphorothioate internucleoside linkage, ‘o’ denotes a phosphate ester linkage; and A, G, C, T denote the relevant nucleosides). All cytosine residues throughout the gapmer are 5-methylcytosines. ISIS 603538 has a target start site of nucleoside 2872 on the rat C9ORF72 mRNA sequence, designated herein as SEQ ID NO: 12 (GENBANK Accession No. NM_001007702.1).

### Mouse Experiment 1

Groups of 4 C57BL/6 mice each were injected with 50 μg, 100 μg, 300 μg, 500 μg, or 700 μg of ISIS 571883 administered via an intracerebroventricular bolus injection. A control group of four C57/BL6 mice were similarly treated with PBS. Animals were anesthetized with 3% isofluorane and placed in a stereotactic frame. After sterilizing the surgical site, each mouse was injected −0.2 mm anterio-posterior from the bregma na d 3 mm dorsoventral to the bregma with the above-mentioned doses of ISIS 571883 using a Hamilton syringe. The incision was closed with sutures. The mice were allowed to recover for 14 days, after which animals were euthanized according to a humane protocol approved by the Institutional Animal Care and Use Committee. Brain and spinal cord tissue were harvested and snap frozen in liquid nitrogen. Prior to freezing, brain tissue was cut transversely five sections using a mouse brain matrix.

### RNA Analysis

RNA was extracted from a 2-3 mm brain section posterior to the injection site, from brain frontal cortex and from the lumbar section of the spinal cord tissue for analysis of C9ORF72 mRNA expression. C9ORF72 mRNA expression was measured by RT-PCR. The data is presented in Table 17. The results indicate that treatment with increasing doses of ISIS 571883 resulted in dose-dependent inhibition of C9ORF72 mRNA expression.

The induction of the microglial marker AIF-1 as a measure of CNS toxicity was also assessed. The data is presented in Table 18. The results indicate that treatment with increasing doses of ISIS 571883 did not result in significant increases in AIF-1 mRNA expression. Hence, the injection of ISIS 571883 was deemed tolerable in this model.

TABLE 17
Percentage inhibition of C9ORF72 mRNA
expression compared to the PBS control

| Dose | Posterior | | Spinal |
|---|---|---|---|
| (μg) | brain | Cortex | cord |
| 50 | 22 | 8 | 46 |
| 100 | 22 | 12 | 47 |
| 300 | 55 | 47 | 67 |
| 500 | 61 | 56 | 78 |
| 700 | 65 | 65 | 79 |

TABLE 18
Percentage expression of AIF-1 mRNA
expression compared to the PBS control

| Dose | Posterior | Spinal |
|---|---|---|
| (μg) | brain | cord |
| 50 | 102 | 89 |
| 100 | 105 | 111 |
| 300 | 107 | 98 |
| 500 | 131 | 124 |
| 700 | 122 | 116 |

### Mouse Experiment 2

Groups of 4 C57BL/6 mice each were injected with 500 μg of ISIS 571883 administered via an intracerebroventricular bolus injection in a procedure similar to that described above. A control group of four C57/BL6 mice were similarly treated with PBS. The mice were tested at regular time points after ICV administration.

### Behavior Analysis

Two standard assays to assess motor behavior were employed; the rotarod assay and grip strength assay. In case of the rotarod assays, the time of latency to fall was measured. The data for the assays is presented in Tables 19 and 20. The results indicate that there were no significant changes in the motor behavior of the mice as a result of antisense inhibition of ISIS 571883 or due to the ICV injection. Hence, antisense inhibition of C9ORF72 was deemed tolerable in this model.

TABLE 19
Latency to fall (sec) in the rotarod assay

| Weeks after | | ISIS |
|---|---|---|
| injection | PBS | 571883 |
| 0 | 66 | 66 |
| 4 | 91 | 70 |
| 8 | 94 | 84 |

TABLE 20
Mean hindlimb grip strength (g) in the grip strength assay

| Weeks after | | ISIS |
|---|---|---|
| injection | PBS | 571883 |
| 0 | 57 | 63 |
| 1 | 65 | 51 |
| 2 | 51 | 52 |
| 3 | 51 | 51 |
| 4 | 59 | 72 |
| 5 | 60 | 64 |
| 6 | 61 | 72 |
| 7 | 67 | 68 |
| 8 | 66 | 70 |
| 9 | 63 | 61 |
| 10 | 48 | 46 |

### Rat Experiment

Groups of 4 Sprague-Dawley rats each were injected with 700 μg, 1,000 μg, or 3,000 μg of ISIS 603538 administered via an intrathecal bolus injection. A control group of four Sprague-Dawley rats were similarly treated with PBS. Animals were anesthetized with 3% isofluorane and placed in a stereotactic frame. After sterilizing the surgical site, each rat was injected with 30 μL of ASO solution administered via 8 cm intrathecal catheter 2 cm into the spinal canal with a 50 μL flush. The rats were allowed to recover for 4 weeks, after which animals were euthanized according to a humane protocol approved by the Institutional Animal Care and Use Committee.

### RNA Analysis

RNA was extracted from a 2-3 mm brain section posterior to the injection site, from brain frontal cortex, and from the cervical and lumbar sections of the spinal cord tissue for analysis of C9ORF72 mRNA expression. C9ORF72 mRNA expression was measured by RT-PCR. The data is presented in Table 21. The results indicate that treatment with increasing doses of ISIS 603538 resulted in dose-dependent inhibition of C9ORF72 mRNA expression.

The induction of the microglial marker AIF-1 as a measure of CNS toxicity was also assessed. The data is presented in Table 22. The results indicate that treatment with increasing doses of ISIS 603538 did not result in significant increases in AIF-1 mRNA expression. Hence, the injection of ISIS 603538 was deemed tolerable in this model.

TABLE 21
Percentage inhibition of C9ORF72 mRNA
expression compared to the PBS control

| Dose | Brain | | Spinal cord | Spinal cord |
|---|---|---|---|---|
| (μg) | (1 mm section) | Cortex | (lumbar) | (cervical) |
| 700 | 21 | 4 | 86 | 74 |
| 1000 | 53 | 49 | 88 | 82 |
| 3000 | 64 | 62 | 88 | 80 |

TABLE 22
Percentage expression of AIF-1 mRNA
expression compared to the PBS control

| Dose | Brain | | Spinal cord | Spinal cord |
|---|---|---|---|---|
| (μg) | (1 mm section) | Cortex | (lumbar) | (cervical) |
| 700 | 97 | 119 | 98 | 89 |
| 1000 | 105 | 113 | 122 | 96 |
| 3000 | 109 | 141 | 156 | 115 |

### Body Weight Analysis

Body weights of the rats were measured at regular time point intervals. The data is presented in Table 23. The results indicate that treatment with increasing doses of ISIS 603538 did not have any significant changes in the body weights of the rats.

TABLE 23
Body weights of the rats (% initial body weight)

| | Dose | | | | | |
|---|---|---|---|---|---|---|
| | (μg) | Week 1 | Week 2 | Week 3 | Week 4 | Week 5 |
| PBS | | 100 | 94 | 103 | 105 | 109 |
| ISIS | 700 | 100 | 94 | 98 | 103 | 107 |
| 603538 | 1000 | 100 | 95 | 97 | 101 | 103 |
| | 3000 | 100 | 92 | 98 | 102 | 105 |

### Example 6: Preferential inhibition of human C90RF72 expression in two patient fibroblast lines

Two different fibroblast cell lines from human patients (F09-152 and F09-229) were analyzed with antisense oligonucleotides that target the C9ORF72 pre-mRNA sequence before exon 1B; i.e. antisense oligonucleotides that target the hexanucleotide repeat expansion containing transcript and antisense oligonucleotides that target downstream of exon 1. The target start and stop sites and the target regions with respect to SEQ ID NOs: 1 and 2 for each oligonucleotide are provided in Table 24. ISIS 577061 and ISIS 577065 target C9ORF72 upstream of exon 1B and just upstream of the hexanucleotide repeat. The rest of the ISIS oligonucleotides of Table 24 target C9ORF72 downstream of exon 1B and the hexanucleotide repeat.

TABLE 24
Target Start and Stop sites of ISIS oligonucleotides used
in a dose response assay in C9ORF72 patient fibroblasts

| | Target | Target | |
|---|---|---|---|
| | Start Site | Start Site | |
| ISIS | at SEQ | at SEQ | |
| No | ID NO: 1 | ID NO: 2 | Target Region |
| 577061 | n/a | 1406 | Upstream of |
| | | | exon 1B |
| 577065 | n/a | 1446 | Upstream of |
| | | | exon 1B |
| 577083 | n/a | 3452 | Downstream |
| | | | of exon 1B |
| 576816 | 232 | 7990 | Exon 2 |
| 576974 | 3132 | 28251 | Exon 11 |

Cells were plated at a density of 20,000 cells per well and transfected using electroporation with 246.9 nM, 740.7 nM, 2,222.2 nM, 6,666.7 nM, and 20,000.0 nM concentrations of antisense oligonucleotide. After a treatment period of approximately 16 hours, RNA was isolated from the cells and C9ORF72 mRNA levels were measured by quantitative real-time PCR. Two primer probe sets were used: (1) human C9ORF72 primer probe set RTS3750, which measures total mRNA levels, and (2) RTS3905, which targets the hexanucleotide repeat expansion containing transcript, which measures only mRNA transcripts that contain the hexanucleotide repeat expansion. C9ORF72 mRNA levels were adjusted according to total RNA content, as measured by RIBOGREEN®. Results are presented as percent inhibition of C9ORF72, relative to untreated control cells.

As illustrated in Table 25, below, the two oligonucleotides that target upstream of exon 1B and, therefore, target mRNA transcripts containing the hexanucleotide repeat expansion (ISIS 577061 and ISIS 577065), do not inhibit total mRNA levels of C9ORF72 (as measured by RTS3750) as well as ISIS 576974, 576816, and 577083, which target downstream of exon 1B and, therefore, do not target the mRNA transcript containing the hexanucleotide repeat expansion. Expression levels of the C9ORF72 mRNA transcript containing the hexanucleotide repeat expansion are low (about 10% of the total C9ORF72 expression products), therefore, oligonucleotides targeting the mRNA transcript containing the hexanucleotide repeat expansion do not robustly inhibit total C9ORF72 mRNA (as measured by RTS3905), as suggested by Table 25 below. Thus. ISIS 577061 and ISIS 577065 preferentially inhibit expression of mRNA transcripts containing the hexanucleotide repeat expansion.

TABLE 25
Percent inhibition of C9ORF72 total mRNA in F09-152 patient
fibroblasts in a dose response assay as measured with RTS3750

| | 246.9 | 740.7 | 2222.2 | 6666.7 | 20000.0 |
|---|---|---|---|---|---|
| ISIS No | nM | nM | nM | nM | nM |
| 577061 | 6 | 11 | 0 | 18 | 10 |
| 577065 | 10 | 11 | 30 | 29 | 0 |
| 576974 | 61 | 69 | 72 | 83 | 83 |
| 576816 | 35 | 76 | 82 | 91 | 93 |
| 577083 | 28 | 38 | 52 | 75 | 80 |

TABLE 26
Percent inhibition of C9ORF72 mRNA transcripts containing
the hexanucleotide repeat expansion in F09-152 patient
fibroblasts in a dose response assay as measured with RTS3905

| | 246.9 | 740.7 | 2222.2 | 6666.7 | 20000.0 |
|---|---|---|---|---|---|
| ISIS No | nM | nM | nM | nM | nM |
| 577061 | 4 | 28 | 58 | 81 | 87 |
| 577065 | 25 | 54 | 70 | 90 | 94 |
| 576974 | 57 | 77 | 81 | 93 | 92 |
| 576816 | 37 | 77 | 91 | 97 | 98 |
| 577083 | 37 | 53 | 74 | 93 | 94 |

TABLE 27
Percent inhibition of C9ORF72 total mRNA in F09-229 patient
fibroblasts in a dose response assay as measured with RTS3750

| | 246.9 | 740.7 | 2222.2 | 6666.7 | 20000.0 |
|---|---|---|---|---|---|
| ISIS No | nM | nM | nM | nM | nM |
| 577061 | 0 | 0 | 0 | 17 | 7 |
| 577065 | 8 | 17 | 17 | 16 | 3 |
| 576974 | 43 | 58 | 85 | 85 | 74 |
| 576816 | 45 | 70 | 85 | 81 | 89 |
| 577083 | 22 | 45 | 56 | 76 | 78 |

TABLE 28
Percent inhibition of C9ORF72 mRNA transcripts containing
the hexanucleotide repeat expansion in F09-229 patient
fibroblasts in a dose response assay as measured with RTS3905

| | 246.9 | 740.7 | 2222.2 | 6666.7 | 20000.0 |
|---|---|---|---|---|---|
| ISIS No | nM | nM | nM | nM | nM |
| 577061 | 14 | 36 | 70 | 87 | 89 |
| 577065 | 26 | 48 | 92 | 91 | 98 |
| 576974 | 63 | 87 | 91 | 92 | 91 |
| 576816 | 62 | 81 | 96 | 98 | 100 |
| 577083 | 36 | 64 | 82 | 98 | 96 |

1-41. (canceled)

42. A compound comprising a modified oligonucleotide consisting of 12 to 50 linked nucleosides and comprising a nucleobase sequence comprising at least 8, at least 9, at least 10, at least 11, at least 12, at least 13, at least 14, at least 15, at least 16, at least 17, at least 18, at least 19, or at least 20 contiguous nucleobases of SEQ ID NO: 30-107.

43. The compound of claim 42, wherein the human C9ORF72 nucleic acid has a nucleobase sequence having at least 85% sequence identity to any one of SEQ ID NOs: 1-10.

44. The compound of claim 42, wherein the modified oligonucleotide is a gapmer.

45. The compound of claim 42, wherein the modified oligonucleotide comprises at least one modified internucleoside linkage.

46. The compound of claim 45, wherein the modified internucleoside linkage is a phosphorothioate internucleoside linkage.

47. The compound of claim 42, wherein the modified oligonucleotide comprises at least one modified nucleoside.

48. The compound of claim 42, wherein the modified oligonucleotide comprises at least one modified nucleoside having a modified sugar.

49. The compound of claim 48, wherein the at least one modified nucleoside having a modified sugar comprises a bicyclic sugar moiety or a non-bicyclic 2′-modified sugar moiety.

50. The compound of claim 49, wherein:
(a) the bicyclic sugar comprises a chemical bridge between the 4′ and 2′ positions of the sugar, wherein the chemical bridge is selected from: 4′-CH(R)—O-2′ and 4′-(CH2)2-O-2′, wherein R is independently H, C₁-C₆ alkyl, and C₁-C₆ alkoxy; or
(b) the non-bicyclic 2′-modified sugar moiety comprises a 2′-O-methoxyethyl group or a 2′-O-methyl group.

51. A compound comprising a single-stranded antisense oligonucleotide consisting of 12 to 30 linked nucleosides and comprising a nucleobase sequence comprising at least 8, at least 9, at least 10, at least 11, at least 12, at least 13, at least 14, at least 15, at least 16, at least 17, at least 18, at least 19, or at least 20 contiguous nucleobases of SEQ ID NO: 180-257.

52. The compound of claim 51, wherein the human C9ORF72 nucleic acid has a nucleobase sequence having at least 85% sequence identity to any one of SEQ ID NOs: 1-10.

53. The compound of claim 51, wherein the modified oligonucleotide is a gapmer.

54. The compound of claim 51, wherein the modified oligonucleotide comprises at least one modified internucleoside linkage.

55. The compound of claim 54, wherein the modified internucleoside linkage is a phosphorothioate internucleoside linkage.

56. The compound of claim 51, wherein the modified oligonucleotide comprises at least one modified nucleoside.

57. The compound of claim 51, wherein the modified oligonucleotide comprises at least one modified nucleoside having a modified sugar.

58. The compound of claim 57, wherein the at least one modified nucleoside having a modified sugar comprises a bicyclic sugar moiety or a non-bicyclic 2′-modified sugar moiety.

59. The compound of claim 58, wherein:
(a) the bicyclic sugar comprises a chemical bridge between the 4′ and 2′ positions of the sugar, wherein the chemical bridge is selected from: 4′-CH(R)13 O-2′ and 4′-(CH2)2-O-2′, wherein R is independently H, C₁-C₆ alkyl, and C₁-C₆ alkoxy; or (b) the non-bicyclic 2′-modified sugar moiety comprises a 2′-O-methoxyethyl group or a 2′-O-methyl group.

60. A method of treating, preventing, or ameliorating a neurodegenerative disease, comprising administering to a subject having or at risk for developing the neurodegenerative disease a therapeutically effective amount of a modified oligonucleotide consisting of 12 to 50 linked nucleosides and having a nucleobase sequence comprising at least 8 contiguous nucleobases complementary to an equal length portion of a human C9ORF72 nucleic acid or an equal length portion of a human C9ORF72 homolog nucleic acid.

61. The method of claim 60, wherein the neurodegenerative disease is any of amyotrophic lateral sclerosis (ALS), frontotemporal dementia (FTD), corticalbasal degeneration syndrome (CBD), atypical Parkinsonian syndrome, and olivopontocellar degeneration (OPCD).


SEQUENCE LISTING (370 sequences)

SEQ ID NO: 1 - RNA, 3339 nt, Homo sapiens
  source 1..3339: mol_type = mRNA
acgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttg

SEQ ID NO: 2 - DNA, 30001 bp, Homo sapiens
  source 1..30001: mol_type = genomic DNA
caaagaaaagggggaggttttgttaaaaaagagaaatgttacatagtgctctttgagaaaattcattggcactattaaggatctgaggagctggtgagtttcaactggtgagtgatggtggtagataaaattagagctgcagcaggtcattttagcaactattagataaaactggtctcaggtcacaacgggcagttgcagcagctggacttggagagaattacactgtgggagcagtgtcatttgtcctaagtgcttttctaccccctacccccactattttagttgggtataaaaagaatgacccaatttgtatgatcaactttcacaaagcatagaacagtaggaaaagggtctgtttctgcagaaggtgtagacgttgagagccattttgtgtatttattcctccctttcttcctcggtgaatgattaaaacgttctgtgtgatttttagtgatgaaaaagattaaatgctactcactgtagtaagtgccatctcacacttgcagatcaaaaggcacacagtttaaaaaacctttgtttttttacacatctgagtggtgtaaatgctactcatctgtagtaagtggaatctatacacctgcagaccaaaagacgcaaggtttcaaaaatctttgtgttttttacacatcaaacagaatggtacgtttttcaaaagttaaaaaaaaacaactcatccacatattgcaactagcaaaaatgacattccccagtgtgaaaatcatgcttgagagaattcttacatgtaaaggcaaaattgcgatgactttgcaggggaccgtgggattcccgcccgcagtgccggagctgtcccctaccagggtttgcagtggagttttgaatgcacttaacagtgtcttacggtaaaaacaaaatttcatccaccaattatgtgttgagcgcccactgcctaccaagcacaaacaaaaccattcaaaaccacgaaatcgtcttcactttctccagatccagcagcctcccctattaaggttcgcacacgctattgcgccaacgctcctccagagcgggtcttaagataaaagaacaggacaagttgccccgccccatttcgctagcctcgtgagaaaacgtcatcgcacatagaaaacagacagacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggaggtgtgtgtttttgtttttcccaccctctctccccactacttgctctcacagtactcgctgagggtgaacaagaaaagacctgataaagattaaccagaagaaaacaaggagggaaacaaccgcagcctgtagcaagctctggaactcaggagtcgcgcgctaggggccggggccggggccggggcgtggtcggggcgggcccgggggcgggcccggggcggggctgcggttgcggtgcctgcgcccgcggcggcggaggcgcaggcggtggcgagtgggtgagtgaggaggcggcatcctggcgggtggctgtttggggttcggctgccgggaagaggcgcgggtagaagcgggggctctcctcagagctcgacgcatttttactttccctctcatttctctgaccgaagctgggtgtcgggctttcgcctctagcgactggtggaattgcctgcatccgggccccgggcttcccggcggcggcggcggcggcggcggcgcagggacaagggatggggatctggcctcttccttgctttcccgccctcagtacccgagctgtctccttcccggggacccgctgggagcgctgccgctgcgggctcgagaaaagggagcctcgggtactgagaggcctcgcctgggggaaggccggagggtgggcggcgcgcggcttctgcggaccaagtcggggttcgctaggaacccgagacggtccctgccggcgaggagatcatgcgggatgagatgggggtgtggagacgcctgcacaatttcagcccaagcttctagagagtggtgatgacttgcatatgagggcagcaatgcaagtcggtgtgctccccattctgtgggacatgacctggttgcttcacagctccgagatgacacagacttgcttaaaggaagtgactattgtgacttgggcatcacttgactgatggtaatcagttgtctaaagaagtgcacagattacatgtccgtgtgctcattgggtctatctggccgcgttgaacaccaccaggctttgtattcagaaacaggagggaggtcctgcactttcccaggaggggtggccctttcagatgcaatcgagattgttaggctctgggagagtagttgcctggttgtggcagttggtaaatttctattcaaacagttgccatgcaccagttgttcacaacaagggtacgtaatctgtctggcattacttctacttttgtacaaaggatcaaaaaaaaaaaagatactgttaagatatgatttttctcagactttgggaaacttttaacataatctgtgaatatcacagaaacaagactatcatataggggatattaataacctggagtcagaatacttgaaatacggtgtcatttgacacgggcattgttgtcaccacctctgccaaggcctgccactttaggaaaaccctgaatcagttggaaactgctacatgctgatagtacatctgaaacaagaacgagagtaattaccacattccagattgttcactaagccagcatttacctgctccaggaaaaaattacaagcaccttatgaagttgataaaatattttgtttggctatgttggcactccacaatttgctttcagagaaacaaagtaaaccaaggaggacttctgtttttcaagtctgccctcgggttctattctacgttaattagatagttcccaggaggactaggttagcctacctattgtctgagaaacttggaactgtgagaaatggccagatagtgatatgaacttcaccttccagtcttccctgatgttgaagattgagaaagtgttgtgaactttctggtactgtaaacagttcactgtccttgaagtggtcctgggcagctcctgttgtggaaagtggacggtttaggatcctgcttctctttgggctgggagaaaataaacagcatggttacaagtattgagagccaggttggagaaggtggcttacacctgtaatgccagagctttgggaggcggaggcaagaggatcacttgaagccaggagttcaagctcaacctgggcaacgtagaccctgtctctacaaaaaattaaaaacttagccgggcgtggtgatgtgcacctgtagtcctagctacttgggaggctgaggcaggagggtcatttgagcccaagagtttgaagttaccgagagctatgatcctgccagtgcattccagcctggatgacaaaacgagaccctgtctctaaaaaacaagaagtgagggctttatgattgtagaattttcactacaatagcagtggaccaaccacctttctaaataccaatcagggaagagatggttgattttttaacagacgtttaaagaaaaagcaaaacctcaaacttagcactctactaacagttttagcagatgttaattaatgtaatcatgtctgcatgtatgggattatttccagaaagtgtattgggaaacctctcatgaaccctgtgagcaagccaccgtctcactcaatttgaatcttggcttccctcaaaagactggctaatgtttggtaactctctggagtagacagcactacatgtacgtaagataggtacataaacaactattggttttgagctgatttttttcagctgcatttgcatgtatggatttttctcaccaaagacgatgacttcaagtattagtaaaataattgtacagctctcctgattatacttctctgtgacatttcatttcccaggctatttcttttggtaggatttaaaactaagcaattcagtatgatctttgtccttcattttctttcttattctttttgtttgtttgtttgtttgtttttttcttgaggcagagtctctctctgtcgcccaggctggagtgcagtggcgccatctcagctcattgcaacctctgccacctccgggttcaagagattctcctgcctcagcctcccgagtagctgggattacaggtgtccaccaccacacccggctaattttttgtatttttagtagaggtggggtttcaccatgttggccaggctggtcttgagctcctgacctcaggtgatccacctgcctcggcctaccaaagagctgggataacaggtgtgacccaccatgcccggcccattttttttttcttattctgttaggagtgagagtgtaactagcagtataatagttcaattttcacaacgtggtaaaagtttccctataattcaatcagattttgctccagggttcagttctgttttaggaaatacttttattttcagtttaatgatgaaatattagagttgtaatattgcctttatgattatccacctttttaacctaaaagaatgaaagaaaaatatgtttgcaatataattttatggttgtatgttaacttaattcattatgttggcctccagtttgctgttgttagttatgacagcagtagtgtcattaccatttcaattcagattacattcctatatttgatcattgtaaactgactgcttacattgtattaaaaacagtggatattttaaagaagctgtacggcttatatctagtgctgtctcttaagactattaaattgatacaacatatttaaaagtaaatattacctaaatgaatttttgaaattacaaatacacgtgttaaaactgtcgttgtgttcaaccatttctgtacatacttagagttaactgttttgccaggctctgtatgcctactcataatatgataaaagcactcatctaatgctctgtaaatagaagtcagtgctttccatcagactgaactctcttgacaagatgtggatgaaattctttaagtaaaattgtttactttgtcatacatttacagatcaaatgttagctcccaaagcaatcatatggcaaagataggtatatcatagtttgcctattagctgctttgtattgctattattataaatagacttcacagttttagacttgcttaggtgaaattgcaattctttttactttcagtcttagataacaagtcttcaattatagtacaatcacacattgcttaggaatgcatcattaggcgattttgtcattatgcaaacatcatagagtgtacttacacaaacctagatagtatagcctttatgtacctaggccgtatggtatagtctgttgctcctaggccacaaacctgtacaactgttactgtactgaatactatagacagttgtaacacagtggtaaatatttatctaaatatatgcaaacagagaaaaggtacagtaaaagtatggtataaaagataatggtatacctgtgtaggccacttaccacgaatggagcttgcaggactagaagttgctctgggtgagtcagtgagtgagtggtgaattaatgtgaaggcctagaacactgtacaccactgtagactataaacacagtacgctgaagctacaccaaatttatcttaacagtttttcttcaataaaaaattataactttttaactttgtaaactttttaattttttaacttttaaaatacttagcttgaaacacaaatacattgtatagctatacaaaaatattttttctttgtatccttattctagaagcttttttctattttctattttaaatttttttttttacttgttagtcgtttttgttaaaaactaaaacacacacactttcacctaggcatagacaggattaggatcatcagtatcactcccttccacctcactgccttccacctccacatcttgtcccactggaaggtttttaggggcaataacacacatgtagctgtcacctatgataacagtgctttctgttgaatacctcctgaaggacttgcctgaggctgttttacatttaacttaaaaaaaaaaaaagtagaaggagtgcactctaaaataacaataaaaggcatagtatagtgaatacataaaccagcaatgtagtagtttattatcaagtgttgtacactgtaataattgtatgtgctatactttaaataacttgcaaaatagtactaagaccttatgatggttacagtgtcactaaggcaatagcatattttcaggtccattgtaatctaatgggactaccatcatatatgcagtctaccattgactgaaacgttacatggcacataactgtatttgcaagaatgatttgttttacattaatatcacataggatgtacctttttagagtggtatgtttatgtggattaagatgtacaagttgagcaaggggaccaagagccctgggttctgtcttggatgtgagcgtttatgttcttctcctcatgtctgttttctcattaaattcaaaggcttgaacgggccctatttagcccttctgttttctacgtgttctaaataactaaagcttttaaattctagccatttagtgtagaactctctttgcagtgatgaaatgctgtattggtttcttggctagcatattaaatatttttatctttgtcttgatacttcaatgtcgttttaaacatcaggatcgggcttcagtattctcataaccagagagttcactgaggatacaggactgtttgcccattttttgttatggctccagacttgtggtatttccatgtctttttttttttttttttttttgaccttttagcggctttaaagtatttctgttgttaggtgttgtattacttttctaagattacttaacaaagcaccacaaactgagtggctttaaacaacagcaatttattctctcacaattctagaagctagaagtccgaaatcaaagtgttgacaggggcatgatcttcaagagagaagactctttccttgcctcttcctggcttctggtggttaccagcaatcctgagtgttcctttcttgccttgtagtttcaacaatccagtatctgccttttgtcttcacatggctgtctaccatttgtctctgtgtctccaaatctctctccttataaacacagcagttattggattaggccccactctaatccagtatgaccccattttaacatgattacacttatttctagataaggtcacattcacgtacaccaagggttaggaattgaacatatctttttgggggacacaattcaacccacaagtgtcagtctctagctgagcctttcccttcctgtttttctcctttttagttgctatgggttaggggccaaatctccagtcatactagaattgcacatggactggatatttgggaatactgcgggtctattctatgagctttagtatgtaacatttaatatcagtgtaaagaagcccttttttaagttatttctttgaatttctaaatgtatgccctgaatataagtaacaagttaccatgtcttgtaaaatgatcatatcaacaaacatttaatgtgcacctactgtgctagttgaatgtctttatcctgataggagataacaggattccacatctttgacttaagaggacaaaccaaatatgtctaaatcatttggggttttgatggatatctttaaattgctgaacctaatcattggtttcatatgtcattgtttagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggtaagtgatttttcagcttattaatcatgttaacctatctgttgaaagcttattttctggtacatataaatcttatttttttaattatatgcagtgaacatcaaacaataaatgttatttattttgcatttaccctattagatacaaatacatctggtctgatacctgtcatcttcatattaactgtggaaggtacgaaatggtagctccacattatagatgaaaagctaaagcttagacaaataaagaaacttttagaccctggattcttcttgggagcctttgactctaataccttttgtttccctttcattgcacaattctgtcttttgcttactactatgtgtaagtataacagttcaaagtaatagtttcataagctgttggtcatgtagcctttggtctctttaacctctttgccaagttcccaggttcataaaatgaggaggttgaatggaatggttcccaagagaattccttttaatcttacagaaattattgttttcctaaatcctgtagttgaatatataatgctatttacatttcagtatagttttgatgtatctaaagaacacattgaattctccttcctgtgttccagtttgatactaacctgaaagtccattaagcattaccagttttaaaaggcttttgcccaatagtaaggaaaaataatatcttttaaaagaataattttttactatgtttgcaggcttacttccttttttctcacattatgaaactcttaaaatcaggagaatcttttaaacaacatcataatgtttaatttgaaaagtgcaagtcattcttttcctttttgaaactatgcagatgttacattgactgttttctgtgaagttatctttttttcactgcagaataaaggttgttttgattttattttgtattgtttatgagaacatgcatttgttgggttaatttcctacccctgcccccattttttccctaaagtagaaagtatttttcttgtgaactaaattactacacaagaacatgtctattgaaaaataagcaagtatcaaaatgttgtgggttgtttttttaaataaattttctcttgctcaggaaagacaagaaaatgtccagaagattatcttagaaggcacagagagaatggaagatcaggtatatgcaaattgcatactgtcaaatgtttttctcacagcatgtatctgtataaggttgatggctacatttgtcaaggccttggagacatacgaataagcctttaatggagcttttatggaggtgtacagaataaactggaggaagatttccatatcttaaacccaaagagttaaatcagtaaacaaaggaaaatagtaattgcatctacaaattaatatttgctccctttttttttctgtttgcccagaataaattttggataacttgttcatagtaaaaataaaaaaaattgtctctgatatgttctttaaggtactacttctcgaacctttccctagaagtagctgtaacagaaggagagcatatgtacccctgaggtatctgtctggggtgtaggcccaggtccacacaatatttcttctaagtcttatgttgtatcgttaagactcatgcaatttacattttattccataactattttagtattaaaatttgtcagtgatatttcttaccctctcctctaggaaaatgtgccatgtttatcccttggctttgaatgcccctcaggaacagacactaagagtttgagaagcatggttacaagggtgtggcttcccctgcggaaactaagtacagactatttcactgtaaagcagagaagttcttttgaaggagaatctccagtgaagaaagagttcttcacttttacttccatttcctcttgtgggtgaccctcaatgctccttgtaaaactccaatattttaaacatggctgttttgcctttctttgcttctttttagcatgaatgagacagatgatactttaaaaaagtaattaaaaaaaaaaacttgtgaaaatacatggccataatacagaacccaatacaatgatctcctttaccaaattgttatgtttgtacttttgtagatagctttccaattcagagacagttattctgtgtaaaggtctgacttaacaagaaaagatttccctttacccaaagaatcccagtccttatttgctggtcaataagcagggtccccaggaatggggtaactttcagcaccctctaacccactagttattagtagactaattaagtaaacttatcgcaagttgaggaaacttagaaccaactaaaattctgcttttactgggattttgttttttcaaaccagaaacctttacttaagttgactactattaatgaattttggtctctcttttaagtgctcttcttaaaaatgttatcttactgctgagaagttcaagtttgggaagtacaaggaggaatagaaacttaagagattttcttttagagcctcttctgtatttagccctgtaggatttttttttttttttttttttttggtgttgttgagcttcagtgaggctattcattcacttatactgataatgtctgagatactgtgaatgaaatactatgtatgcttaaacctaagaggaaatattttcccaaaattattcttcccgaaaaggaggagttgccttttgattgagttcttgcaaatctcacaacgactttattttgaacaatactgtttggggatgatgcattagtttgaaacaacttcagttgtagctgtcatctgataaaattgcttcacagggaaggaaatttaacacggatctagtcattattcttgttagattgaatgtgtgaattgtaattgtaaacaggcatgataattattactttaaaaactaaaaacagtgaatagttagttgtggaggttactaaaggatggtttttttttaaataaaactttcagcattatgcaaatgggcatatggcttaggataaaacttccagaagtagcatcacatttaaattctcaagcaacttaataatatggggctctgaaaaactggttaaggttactccaaaaatggccctgggtctgacaaagattctaacttaaagatgcttatgaagactttgagtaaaatcatttcataaaataagtgaggaaaaacaactagtattaaattcatcttaaataatgtatgatttaaaaaatatgtttagctaaaaatgcatagtcatttgacaatttcatttatatctcaaaaaatttacttaaccaagttggtcacaaaactgatgagactggtggtggtagtgaataaatgagggaccatccatatttgagacactttacatttgtgatgtgttatactgaattttcagtttgattctatagactacaaatttcaaaattacaatttcaagatgtaataagtagtaatatcttgaaatagctctaaagggaatttttctgttttattgattcttaaaatatatgtgctgattttgatttgcatttgggtagattatacttttatgagtatggaggttaggtattgattcaagttttccttacctatttggtaaggatttcaaagtctttttgtgcttggttttcctcatttttaaatatgaaatatattgatgacctttaacaaattttttttatctcaaattttaaaggagatcttttctaaaagaggcatgatgacttaatcattgcatgtaacagtaaacgataaaccaatgattccatactctctaaagaataaaagtgagctttagggccgggcatggtcagaaatttgacaccaacctggccaacatggcgaaaccccgtctctactaaaaatacaaaaatcagccgggcatggtggcggcacctatagtcccagctacttgggaggatgagacaggagagtcacttgaacctgggaggagaggttgcagtgagctgagatcacgccattgcactccagcctgagcaatgaaagcaaaactccatctcaaaaaaaaaaaaagaaaagaaagaataaaagtgagctttggattgcatataaatcctttagacatgtagtagacttgtttgatactgtgtttgaacaaattacgaagtattttcatcaaagaatgttattgtttgatgttatttttattttttattgcccagcttctctcatattacgtgattttcttcacttcatgtcactttattgtgcagggtcagagtattattccaatgcttactggagaagtgattcctgtaatggaactgctttcatctatgaaatcacacagtgttcctgaagaaatagatgtaagtttaaatgagagcaattatacactttatgagttttttggggttatagtattattatgtatattattaatattctaattttaatagtaaggactttgtcatacatactattcacatacagtattagccactttagcaaataagcacacacaaaatcctggattttatggcaaaacagaggcatttttgatcagtgatgacaaaattaaattcattttgtttatttcattacttttataattcctaaaagtgggaggatcccagctcttataggagcaattaatatttaatgtagtgtcttttgaaacaaaactgtgtgccaaagtagtaaccattaatggaagtttacttgtagtcacaaatttagtttccttaatcatttgttgaggacgttttgaatcacacactatgagtgttaagagatacctttaggaaactattcttgttgttttctgattttgtcatttaggttagtctcctgattctgacagctcagaagaggaagttgttcttgtaaaaattgtttaacctgcttgaccagctttcacatttgttcttctgaagtttatggtagtgcacagagattgttttttggggagtcttgattctcggaaatgaaggcagtgtgttatattgaatccagacttccgaaaacttgtatattaaaagtgttatttcaacactatgttacagccagactaatttttttattttttgatgcattttagatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggctcaataagaaaatttctgtgaaaggtctctagaaaatgtaggttcctatacaaataaaagataacattgtgcttatagcttcggtgtttatcatataaagctattctgagttatttgaagagctcacctacttttttttgtttttagtttgttaaattgttttataggcaatgtttttaatctgttttctttaacttacagtgccatcagctcacacttgcaaacctgtggctgttccgttgtagtaggtagcagtgcagagaaagtaaataaggtagtttattttataatctagcaaatgatttgactctttaagactgatgatatatcatggattgtcatttaaatggtaggttgcaattaaaatgatctagtagtataaggaggcaatgtaatctcatcaaattgctaagacaccttgtggcaacagtgagtttgaaataaactgagtaagaatcatttatcagtttattttgatagctcggaaataccagtgtcagtagtgtataaatggttttgagaatatattaaaatcagatatataaaaaaaattactcttctatttcccaatgttatctttaacaaatctgaagatagtcatgtacttttggtagtagttccaaagaaatgttatttgtttattcatcttgatttcattgtcttcgctttccttctaaatctgtcccttctagggagctattgggattaagtggtcattgattattatactttattcagtaatgtttctgaccctttccttcagtgctacttgagttaattaaggattaatgaacagttacatttccaagcattagctaataaactaaaggattttgcacttttcttcactgaccattagttagaaagagttcagagataagtatgtgtatctttcaatttcagcaaacctaattttttaaaaaaagttttacataggaaatatgttggaaatgatactttacaaagatattcataatttttttttgtaatcagctactttgtatatttacatgagccttaatttatatttctcatataaccatttatgagagcttagtatacctgtgtcattatattgcatctacgaactagtgaccttattccttctgttacctcaaacaggtggctttccatctgtgatctccaaagccttaggttgcacagagtgactgccgagctgctttatgaagggagaaaggctccatagttggagtgtttttttttttttttttaaacatttttcccatcctccatcctcttgagggagaatagcttaccttttatcttgttttaatttgagaaagaagttgccaccactctaggttgaaaaccactcctttaacataataactgtggatatggtttgaatttcaagatagttacatgcctttttatttttcctaatagagctgtaggtcaaatattattagaatcagatttctaaatcccacccaatgacctgcttattttaaatcaaattcaataattaattctcttctttttggaggatctggacattctttgatatttcttacaacgaatttcatgtgtagacccactaaacagaagctataaaagttgcatggtcaaataagtctgagaaagtctgcagatgatataattcacctgaagagtcacagtatgtagccaaatgttaaaggttttgagatgccatacagtaaatttaccaagcattttctaaatttatttgaccacagaatccctattttaagcaacaactgttacatcccatggattccaggtgactaaagaatacttatttcttaggatatgttttattgataataacaattaaaatttcagatatctttcataagcaaatcagtggtctttttacttcatgttttaatgctaaaatattttcttttatagatagtcagaacattatgcctttttctgactccagcagagagaaaatgctccaggttatgtgaagcagaatcatcatttaaatatgagtcagggctctttgtacaaggcctgctaaaggtatagtttctagttatcacaagtgaaaccacttttctaaaatcatttttgagactctttatagacaaatcttaaatattagcatttaatgtatctcatattgacatgcccagagactgacttcctttacacagttctgcacatagactatatgtcttatggatttatagttagtatcatcagtgaaacaccatagaataccctttgtgttccaggtgggtccctgttcctacatgtctagcctcaggacttttttttttttaacacatgcttaaatcaggttgcacatcaaaaataagatcatttctttttaactaaatagatttgaattttattgaaaaaaaattttaaacatctttaagaagcttataggatttaagcaattcctatgtatgtgtactaaaatatatatatttctatatataatatatattagaaaaaaattgtatttttcttttatttgagtctactgtcaaggagcaaaacagagaaatgtaaattagcaattatttataatacttaaagggaagaaagttgttcaccttgttgaatctattattgttatttcaattatagtcccaagacgtgaagaaatagctttcctaatggttatgtgattgtctcatagtgactactttcttgaggatgtagccacggcaaaatgaaataaaaaaatttaaaaattgttgcaaatacaagttatattaggcttttgtgcattttcaataatgtgctgctatgaactcagaatgatagtatttaaatatagaaactagttaaaggaaacgtagtttctatttgagttatacatatctgtaaattagaacttctcctgttaaaggcataataaagtgcttaatacttttgtttcctcagcaccctctcatttaattatataattttagttctgaaagggacctataccagatgcctagaggaaatttcaaaactatgatctaatgaaaaaatatttaatagttctccatgcaaatacaaatcatatagttttccagaaaatacctttgacattatacaaagatgattatcacagcattataatagtaaaaaaatggaaatagcctctttcttctgttctgttcatagcacagtgcctcatacgcagtaggttattattacatggtaactggctaccccaactgattaggaaagaagtaaatttgttttataaaaatacatactcattgaggtgcatagaataattaagaaattaaaagacacttgtaattttgaatccagtgaatacccactgttaatatttggtatatctctttctagtctttttttcccttttgcatgtattttctttaagactcccacccccactggatcatctctgcatgttctaatctgcttttttcacagcagattctaagcctctttgaatatcaacacaaacttcaacaacttcatctatagatgccaaataataaattcatttttatttacttaaccacttcctttggatgcttaggtcattctgatgttttgctattgaaaccaatgctatactgaacacttctgtcactaaaactttgcacacactcatgaatagcttcttaggataaatttttagagatggatttgctaaatcagagaccattttttaaaattaaaaaacaattattcatatcgtttggcatgtaagacagtaaattttccttttattttgacaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgtacgtaatgctctgcctgctggtactgtagtcaagcaatatgaaattgtgtcttttacgaataaaaacaaaacagaagttgcatttaaaaagaaagaaatattaccagcagaattatgcttgaagaaacatttaatcaagcatttttttcttaaatgttcttctttttccatacaattgtgtttaccctaaaataggtaagattaacccttaaagtaaatatttaactatttgtttaataaatatatattgagctcctaggcactgttctaggtaccgggcttaatagtggccaaccagacagccccagccccagcccctacattgtgtatagtctattatgtaacagttattgaatggacttattaacaaaaccaaagaagtaattctaagtcttttttttcttgacatatgaatataaaatacagcaaaactgttaaaatatattaatggaacatttttttactttgcattttatattgttattcacttcttatttttttttaaaaaaaaaagcctgaacagtaaattcaaaaggaaaagtaatgataattaattgttgagcatggacccaacttgaaaaaaaaaatgatgatgataaatctataatcctaaaaccctaagtaaacacttaaaagatgttctgaaatcaggaaaagaattatagtatacttttgtgtttctcttttatcagttgaaaaaaggcacagtagctcatgcctgtaagaacagagctttgggagtgcaaggcaggcggatcacttgaggccaggagttccagaccagcctgggcaacatagtgaaaccccatctctacaaaaaataaaaaagaattattggaatgtgtttctgtgtgcctgtaatcctagctattccgaaagctgaggcaggaggatcttttgagcccaggagtttgaggttacagggagttatgatgtgccagtgtactccagcctggggaacaccgagactctgtcttatttaaaaaaaaaaaaaaaaaaatgcttgcaataatgcctggcacatagaaggtaacagtaagtgttaactgtaataacccaggtctaagtgtgtaaggcaatagaaaaattggggcaaataagcctgacctatgtatctacagaatcagtttgagcttaggtaacagacctgtggagcaccagtaattacacagtaagtgttaaccaaaagcatagaataggaatatcttgttcaagggacccccagccttatacatctcaaggtgcagaaagatgacttaatataggacccattttttcctagttctccagagtttttattggttcttgagaaagtagtaggggaatgttttagaaaatgaattggtccaactgaaattacatgtcagtaagtttttatatattggtaaattttagtagacatgtagaagttttctaattaatctgtgccttgaaacattttcttttttcctaaagtgcttagtattttttccgttttttgattggttacttgggagcttttttgaggaaatttagtgaactgcagaatgggtttgcaaccatttggtatttttgttttgttttttagaggatgtatgtgtattttaacatttcttaatcatttttagccagctatgtttgttttgctgatttgacaaactacagttagacagctattctcattttgctgatcatgacaaaataatatcctgaatttttaaattttgcatccagctctaaattttctaaacataaaattgtccaaaaaatagtattttcagccactagattgtgtgttaagtctattgtcacagagtcattttacttttaagtatatgtttttacatgttaattatgtttgttatttttaattttaactttttaaaataattccagtcactgccaatacatgaaaaattggtcactggaattttttttttgacttttattttaggttcatgtgtacatgtgcaggtgtgttatacaggtaaattgcgtgtcatgagggtttggtgtacaggtgatttcattacccaggtaataagcatagtacccaataggtagttttttgatcctcacccttctcccaccctcaagtaggccctggtgttgctgtttccttctttgtgtccatgtatactcagtgtttagctcccacttagaagtgagaacatgcggtagttggttttctgttcctggattagttcacttaggataatgacctctagctccatctggtttttatggctgcatagtattccatggtgtatatgtatcacattttctttatccagtctaccattgataggcatttaggttgattccctgtctttgttatcatgaatagtgctgtgatgaacatacacatgcatgtgtctttatggtagaaaaatttgtattcctttaggtacatatagaataatggggttgctagggtgaatggtagttctattttcagttatttgagaaatcttcaaactgcttttcataatagctaaactaatttacagtcccgccagcagtgtataagtgttcccttttctccacaaccttgccaacatctgtgattttttgactttttaataatagccattcctagagaattgatttgcaattctctattagtgatattaagcattttttcatatgctttttagctgtctgtatatattcttctgaaaaattttcatgtcctttgcccagtttgtagtggggtgggttgttttttgcttgttaattagttttaagttccttccagattctgcatatccctttgttggatacatggtttgcagatatttttctcccattgtgtaggttgtcttttactctgttgatagtttcttttgccatgcaggagctcgttaggtcccatttgtgtttgtttttgttgcagttgcttttggcgtcttcatcataaaatctgtgccagggcctatgtccagaatggtatttcctaggttgtcttccagggtttttacaattttagattttacgtttatgtctttaatccatcttgagttgatttttgtatatggcacaaggaaggggtccagtttcactccaattcctatggctagcaattatcccagcaccatttattgaatacggagtcctttccccattgcttgttttttgtcaactttgttgaagatcagatggttgtaagtgtgtggctttatttcttggctctctattctccattggtctatgtgtctgtttttataacagtaccctgctgttcaggttcctatagccttttagtataaaatcggctaatgtgatgcctccagctttgttctttttgcttaggattgctttggctatttgggctcctttttgggtccatattaattttaaaacagttttttctggttttgtgaaggatatcattggtagtttataggaatagcattgaatctgtagattgctttgggcagtatggccattttaacaatattaattcttcctatctatgaatatggaatgtttttccatgtgtttgtgtcatctctttatacctgatgtataaagaaaagctggtattattcctactcaatctgttccaaaaaattgaggaggaggaactcttccctaatgaggccagcatcattctgataccaaaacctggcagagacacaacagaaaaaagaaaacttcaggccaatatccttgatgaatatagatgcaaaaatcctcaacaaaatactagcaaaccaaatccagcagcacatcaaaaagctgatctactttgatcaagtaggctttatccctgggatgcaaggttggttcaacatacacaaatcaataagtgtgattcatcacataaacagagctaaaaacaaaaaccacaagattatctcaataggtagagaaaaggttgtcaataaaatttaacatcctccatgttaaaaaccttcagtaggtcaggtgtagtgactcacacctgtaatcccagcactttgggaggccaaggcgggcatatctcttaagcccaggagttcaagacgagcctaggcagcatggtgaaaccccatctctacaaaaaaaaaaaaaaaaaaaaattagcttggtatggtgacatgcacctatagtcccagctattcaggaggttgaggtgggaggattgtttgagcccgggaggcagaggttggcagcgagctgagatcatgccaccgcactccagcctgggcaacggagtgagaccctgtctcaaaaaagaaaaatcacaaacaatcctaaacaaactaggcattgaaggaacatgcctcaaaaaaataagaaccatctatgacagacccatagccaatatcttaccaaatgggcaaaagctggaagtattctccttgagaaccgtaacaagacaaggatgtccactctcaccactccttttcagcatagttctggaagtcctagccagagcaatcaggaaagagaaagaaagaaagacattcagataggaagagaagaagtcaaactatttctgtttgcaggcagtataattctgtacctagaaaatctcatagtctctgcccagaaactcctaaatctgttaaaaatttcagcaaagttttggcattctctatactccaacaccttccaaagtgagagcaaaatcaagaacacagtcccattcacaatagccgcaaaacgaataaaatacctaggaatccagctaaccagggaggtgaaagatctctatgagaattacaaaacactgctgaaagaaatcagagatgacacaaacaaatggaaatgttcttttttaacaccttgctttatctaattcacttatgatgaagatactcattcagtggaacaggtataataagtccactcgattaaatataagccttattctctttccagagcccaagaaggggcactatcagtgcccagtcaataatgacgaaatgctaatatttttcccctttacggtttctttcttctgtagtgtggtacactcgtttcttaagataaggaaacttgaactaccttcctgtttgcttctacacatacccattctctttttttgccactctggtcaggtataggatgatccctaccactttcagttaaaaactcctcctcttactaaatgttctcttaccctctggcctgagtagaacctagggaaaatggaagagaaaaagatgaaagggaggtggggcctgggaagggaataagtagtcctgtttgtttgtgtgtttgctttagcacctgctatatcctaggtgctgtgttaggcacacattattttaagtggccattatattactactactcactctggtcgttgccaaggtaggtagtactttcttggatagttggttcatgttacttacagatggtgggcttgttgaggcaaacccagtggataatcatcggagtgtgttctctaatctcactcaaatttttcttcacattttttggtttgttttggtttttgatggtagtggcttatttttgttgctggtttgttttttgtttttttttgagatggcaagaattggtagttttatttattaattgcctaagggtctctactttttttaaaagatgagagtagtaaaatagattgatagatacatacatacccttactggggactgcttatattctttagagaaaaaattacatattagcctgacaaacaccagtaaaatgtaaatatatccttgagtaaataaatgaatgtatattttgtgtctccaaatatatatatctatattcttacaaatgtgtttatatgtaatatcaatttataagaacttaaaatgttggctcaagtgagggattgtggaaggtagcattatatggccatttcaacatttgaacttttttcttttcttcattttcttcttttcttcaggaatatttttcaagatgtcttacacagagacactctagtgaaagccttcctggatcaggtaaatgttgaacttgagattgtcagagtgaatgatatgacatgttttcttttttaatatatcctacaatgcctgttctatatatttatattcccctggatcatgccccagagttctgctcagcaattgcagttaagttagttacactacagttctcagaagagtctgtgagggcatgtcaagtgcatcattacattggttgcctcttgtcctagatttatgcttcgggaattcagacctttgtttacaatataataaatattattgctatcttttaaagatataataataagatataaagttgaccacaactactgttttttgaaacatagaattcctggtttacatgtatcaaagtgaaatctgacttagcttttacagatataatatatacatatatatatcctgcaatgcttgtactatatatgtagtacaagtatatatatatgtttgtgtgtgtatatatatatagtacgagcatatatacatattaccagcattgtaggatatatatatgtttatatattaaaaaaaagttataaacttaaaaccctattatgttatgtagagtatatgttatatatgatatgtaaaatatataacatatactctatgatagagtgtaatatattttttatatatattttaacatttataaaatgatagaattaagaattgagtcctaatctgttttattaggtgctttttgtagtgtctggtctttctaaagtgtctaaatgatttttccttttgacttattaatggggaagagcctgtatattaacaattaagagtgcagcattccatacgtcaaacaacaaacattttaattcaagcattaacctataacaagtaagttttttttttttttttgagaaagggaggttgtttatttgcctgaaatgactcaaaaatatttttgaaacatagtgtacttatttaaataacatctttattgtttcattcttttaaaaaatatctacttaattacacagttgaaggaaatcgtagattatatggaacttatttcttaatatattacagtttgttataataacattctggggatcaggccaggaaactgtgtcatagataaagctttgaaataatgagatccttatgtttactagaaattttggattgagatctatgaggtctgtgacatattgcgaagttcaaggaaaattcgtaggcctggaatttcatgcttctcaagctgacataaaatccctcccactctccacctcatcatatgcacacattctactcctacccacccactccaccccctgcaaaagtacaggtatatgaatgtctcaaaaccataggctcatcttctaggagcttcaatgttatttgaagatttgggcagaaaaaattaagtaatacgaaataacttatgtatgagttttaaaagtgaagtaaacatggatgtattctgaagtagaatgcaaaatttgaatgcatttttaaagataaattagaaaacttctaaaaactgtcagattgtctgggcctggtggcttatgcctgtaatcccagcactttgggagtccgaggtgggtggatcacaaggtcaggagatcgagaccatcctgccaacatggtgaaaccccgtctctactaagtatacaaaaattagctgggcgtggcagcgtgtgcctgtaatcccagctacctgggaggctgaggcaggagaatcgcttgaacccaggaggtgtaggttgcagtgagtcaagatcgcgccactgcactttagcctggtgacagagctagactccgtctcaaaaaaaaaaaaaaatatcagattgttcctacacctagtgcttctataccacactcctgttagggggcatcagtggaaatggttaaggagatgtttagtgtgtattgtctgccaagcactgtcaacactgtcatagaaacttctgtacgagtagaatgtgagcaaattatgtgttgaaatggttcctctccctgcaggtctttcagctgaaacctggcttatctctcagaagtactttccttgcacagtttctacttgtccttcacagaaaagccttgacactaataaaatatatagaagacgatacgtgagtaaaactcctacacggaagaaaaacctttgtacattgtttttttgttttgtttcctttgtacattttctatatcataatttttgcgcttcttttttttttttttttttttttttttccattatttttaggcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttgcctctttcagttgttcattcagaaaaaaatactatgatatttgaagactgatcagcttctgttcagctgacagtcatgctggatctaaactttttttaaaattaattttgtcttttcaaagaaaaaatatttaaagaagctttataatataatcttatgttaaaaaaactttctgcttaactctctggatttcattttgatttttcaaattatatattaatatttcaaatgtaaaatactatttagataaattgtttttaaacattcttattattataatattaatataacctaaactgaagttattcatcccaggtatctaatacatgtatccaaagtaaaaatccaaggaatctgaacactttcatctgcaaagctaggaataggtttgacattttcactccaagaaaaagtttttttttgaaaatagaatagttgggatgagaggtttctttaaaagaagactaactgatcacattactatgattctcaaagaagaaaccaaaacttcatataatactataaagtaaatataaaatagttccttctatagtatatttctataatgctacagtttaaacagatcactcttatataatactattttgattttgatgtagaattgcacaaattgatatttctcctatgatctgcagggtatagcttaaagtaacaaaaacagtcaaccacctccatttaacacacagtaacactatgggactagttttattacttccattttacaaatgaggaaactaaagcttaaagatgtgtaatacaccgcccaaggtcacacagctggtaaaggtggatttcatcccagacagttacagtcattgccatgggcacagctcctaacttagtaactccatgtaactggtactcagtgtagctgaattgaaaggagagtaaggaagcaggttttacaggtctacttgcactattcagagcccgagtgtgaatccctgctgtgctgcttggagaagttacttaacctatgcaaggttcattttgtaaatattggaaatggagtgataatacgtacttcaccagaggatttaatgagaccttatacgatccttagttcagtacctgactagtgcttcataaatgctttttcatccaatctgacaatctccagcttgtaattggggcatttagaacatttaatatgattattggcatggtaggttaaagctgtcatcttgctgttttctatttgttctttttgttttctccttacttttggatttttttattctactatgtcttttctattgtcttattaactatactctttgatttattttagtggttgttttagggttatacctctttctaatttaccagtttataaccagtttatatactacttgacatatagcttaagaaacttactgttgttgtctttttgctgttatggtcttaacgtttttatttctacaaacattataaactccacactttattgttttttaattttacttatacagtcaattatcttttaaagatatttaaatataaacattcaaaacaccccaat

SEQ ID NO: 3 - RNA, 1031 nt, Homo sapiens
  source 1..1031: mol_type = mRNA
attcccgggatacgtaacctacggtgtcccgctaggaaagagaggtgcgtcaaacagcgacaagttccgcccacgtaaaagatgacgcttggtgtgtcagccgtccctgctgcccggttgcttctcttttgggggcggggtctagcaagagcaggtgtgggtttaggagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaaatgtccagaagattatcttagaaggcacagagagaatggaagatcagggtcagagtattattccaatgcttactggagaagtgattcctgtaatggaaactgctttcctctatgaaattcccccgggttcctggaggaaatagatataggctgatacagttacccaatgatggatgaatattgggggaccgcctggtcattgaaaggctttcttttctccaggaaagaaatttttttccttttccataaaaagcttgggaatggaagacaacaattcccattctttttttgcgttccacccctatgtgacaacagaaatttttggggaaacaacaacgaaaaaattttatcccgcgcgca

SEQ ID NO: 4 - RNA, 3244 nt, Homo sapiens
  source 1..3244: mol_type = mRNA
gggcggggctgcggttgcggtgcctgcgcccgcggcggcggaggcgcaggcggtggcgagtggatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaatgccatcagctcacacttgcaaacctgtggctgttccgttgtagtaggtagcagtgcagagaaagtaaataagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcaggatacgatcatctacactgacgaaagctttactcctgatttgaatatttttcaagatgtcttacacagagacactctagtgaaagccttcctggatcaggtctttcagctgaaacctggcttatctctcagaagtactttccttgcacagtttctacttgtccttcacagaaaagccttgacactaataaaatatatagaagacgatacgcagaagggaaaaaagccctttaaatctcttcggaacctgaagatagaccttgatttaacagcagagggcgatcttaacataataatggctctggctgagaaaattaaaccaggcctacactcttttatctttggaagacctttctacactagtgtgcaagaacgagatgttctaatgactttttaaatgtgtaacttaataagcctattccatcacaatcatgatcgctggtaaagtagctcagtggtgtggggaaacgttcccctggatcatactccagaattctgctctcagcaattgcagttaagtaagttacactacagttctcacaagagcctgtgaggggatgtcaggtgcatcattacattgggtgtctcttttcctagatttatgcttttgggatacagacctatgtttacaatataataaatattattgctatcttttaaagatataataataggatgtaaacttgaccacaactactgtttttttgaaatacatgattcatggtttacatgtgtcaaggtgaaatctgagttggcttttacagatagttgactttctatcttttggcattctttggtgtgtagaattactgtaatacttctgcaatcaactgaaaactagagcctttaaatgatttcaattccacagaaagaaagtgagcttgaacataggatgagctttagaaagaaaattgatcaagcagatgtttaattggaattgattattagatcctactttgtggatttagtccctgggattcagtctgtagaaatgtctaatagttctctatagtccttgttcctggtgaaccacagttagggtgttttgtttattttattgttcttgctattgttgatattctatgtagttgagctctgtaaaaggaaattgtattttatgttttagtaattgttgccaactttttaaattaattttcattatttttgagccaaattgaaatgtgcacctcctgtgccttttttctccttagaaaatctaattacttggaacaagttcagatttcactggtcagtcattttcatcttgttttcttcttgctaagtcttaccatgtacctgctttggcaatcattgcaactctgagattataaaatgccttagagaatatactaactaataagatctttttttcagaaacagaaaatagttccttgagtacttccttcttgcatttctgcctatgtttttgaagttgttgctgtttgcctgcaataggctataaggaatagcaggagaaattttactgaagtgctgttttcctaggtgctactttggcagagctaagttatcttttgttttcttaatgcgtttggaccattttgctggctataaaataactgattaatataattctaacacaatgttgacattgtagttacacaaacacaaataaatattttatttaaaattctggaagtaatataaaagggaaaatatatttataagaaagggataaaggtaatagagcccttctgccccccacccaccaaatttacacaacaaaatgacatgttcgaatgtgaaaggtcataatagctttcccatcatgaatcagaaagatgtggacagcttgatgttttagacaaccactgaactagatgactgttgtactgtagctcagtcatttaaaaaatatataaatactaccttgtagtgtcccatactgtgttttttacatggtagattcttatttaagtgctaactggttattttctttggctggtttattgtactgttatacagaatgtaagttgtacagtgaaataagttattaaagcatgtgtaaacattgttatatatcttttctcctaaatggagaattttgaataaaatatatttgaaattttg

SEQ ID NO: 5 - RNA, 761 nt, Homo sapiens
  source 1..761: mol_type = mRNA
cacgaggctttgatatttcttacaacgaatttcatgtgtagacccactaaacagaagctataaaagttgcatggtcaaataagtctgagaaagtctgcagatgatataattcacctgaagagtcacagtatgtagccaaatgttaaaggttttgagatgccatacagtaaatttaccaagcattttctaaatttatttgaccacagaatccctattttaagcaacaactgttacatcccatggattccaggtgactaaagaatacttatttcttaggatatgttttattgataataacaattaaaatttcagatatctttcataagcaaatcagtggtctttttacttcatgttttaatgctaaaatattttcttttatagatagtcagaacattatgcctttttctgactccagcagagagaaaatgctccaggttatgtgaagcagaatcatcatttaaatatgagtcagggctctttgtacaaggcctgctaaaggattcaactggaagctttgtgctgcctttccggcaagtcatgtatgctccatatcccaccacacacatagatgtggatgtcaatactgtgaagcagatgccaccctgtcatgaacatatttataatcagcgtagatacatgagatccgagctgacagccttctggagagccacttcagaagaagacatggctcangatacgatcatctacactgacgaaagctntactcctgatttgaatatttttcaagatgtcttacacag

SEQ ID NO: 6 - RNA, 1901 nt, Homo sapiens
  source 1..1901: mol_type = mRNA
acgtaacctacggtgtcccgctaggaaagagaggtgcgtcaaacagcgacaagttccgcccacgtaaaagatgacgcttgatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatggaactgctttcatctatgaaatcacacagtgttcctgaagaaatagatatagctgatacagtactcaatgatgatgatattggtgacagctgtcatgaaggctttcttctcaagtaagaatttttcttttcataaaagctggatgaagcagataccatcttatgctcacctatgacaagatttggaagaaagaaaataacagactgtctacttagattgttctagggacattacgtatttgaactgttgcttaaatttgtgttatttttcactcattatatttctatatatatttggtgttattccatttgctatttaaagaaaccgagtttccatcccagacaagaaatcatggccccttgcttgattctggtttcttgttttacttctcattaaagctaacagaatcctttcatattaagttgtactgtagatgaacttaagttatttaggcgtagaacaaaattattcatatttatactgatctttttccatccagcagtggagtttagtacttaagagtttgtgcccttaaaccagactccctggattaatgctgtgtacccgtgggcaaggtgcctgaattctctatacacctatttcctcatctgtaaaatggcaataatagtaatagtacctaatgtgtagggttgttataagcattgagtaagataaataatataaagcacttagaacagtgcctggaacataaaaacacttaataatagctcatagctaacatttcctatttacatttcttctagaaatagccagtatttgttgagtgcctacatgttagttcctttactagttgctttacatgtattatcttatattctgttttaaagtttcttcacagttacagattttcatgaaattttacttttaataaaagagaagtaaaagtataaagtattcacttttatgttcacagtcttttcctttaggctcatgatggagtatcagaggcatgagtgtgtttaacctaagagccttaatggcttgaatcagaagcactttagtcctgtatctgttcagtgtcagcctttcatacatcattttaaatcccatttgactttaagtaagtcacttaatctctctacatgtcaatttcttcagctataaaatgatggtatttcaataaataaatacattaattaaatgatattatactgactaattgggctgttttaaggcaaaaaaaaaaaaaaaaaaaaaaaaaaaa

SEQ ID NO: 7 - RNA, 562 nt, Homo sapiens
  source 1..562: mol_type = mRNA
agacgtaacctacggtgtcccgctaggaaagagagatatctccggagcatttggataatgtgacagttggaatgcagtgatgtcgactctttgcccaccgccatctccagctgttgccaagacagagattgctttaagtggcaaatcacctttattagcagctacnttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaagg

SEQ ID NO: 8 - RNA, 798 nt, Homo sapiens
  source 1..798: mol_type = mRNA
gggctctcttttgggggcggggtctagcaagagcagatatctccggagcatttggataatgtgacagttggaatgcagtgatgtcgactctttgcccaccgccatctccagctgttgccaagacagagattgctttaagtggcaaatcacctttattagcagctacttttgcttactgggacaatattcttggtcctagagtaaggcacatttgggctccaaagacagaacaggtacttctcagtgatggagaaataacttttcttgccaaccacactctaaatggagaaatccttcgaaatgcagagagtggtgctatagatgtaaagttttttgtcttgtctgaaaagggagtgattattgtttcattaatctttgatggaaactggaatggggatcgcagcacatatggactatcaattatacttccacagacagaacttagtttctacctcccacttcatagagtgtgtgttgatagattaacacatataatccggaaaggaagaatatggatgcataaggaaagacaagaaaatgtccagaagattatcttagaaggcacagagagaatggaagatcagggtcagagtattattccaatgcttactggagaagtgattcctgtaatgggactgctttcatctatgaaatcacacagtgttcctgaagaaatagatatagctgatacagtactccatgatgatgatatttggtgacagctgtcatgaaaggctttcttctcaagtaggaattttttcttttcataaaagctgggatgaagccagattcccatct

SEQ ID NO: 9 - RNA, 169 nt, Homo sapiens
  source 1..169: mol_type = mRNA
aaacagcgacaagttccgcccacgtaaaagatgatgcttggtgtgtcagccgtccctgctgcccggttgcttctcttttgggggcggggtctagcaagagcagatatctccggagcatttggataatgtgacagttggaatgcggtgatgtcgactctttgcccaccgc

SEQ ID NO: 10 - RNA, 176 nt, Homo sapiens
  source 1..176: mol_type = mRNA
aaaacgtcatcgcacatagaaaacagacagacgtaacctacggtgtcccgctaggaaagagaggtgcgtcaaacagcgacaagttccgcccacgtaaaagatgacgcttgatatctccggagcatttggataatgtgacagttggaatgcagtgatgtcgactctttgcccaccgc

SEQ ID NO: 11 - DNA, 38001 bp, Mus musculus
  source 1..38001: mol_type = genomic DNA
caaacacacacacacacacacacacacacacacacacacacacacacacacacacactggcatatcaagtctctgttaggctaggcgcatcctctcccactgaggtcagacaagactgcccagctagaagaacatatcccacggacaggcaacagcttttgggacagccacgctccagttgtttgggactcataaaagactaaactcacacctgctacaaaagtgcagggaggcctaggtccagcctgtgtgtgctctttgcttagtggtgtctctgagagccctaaggatcaaagtttgttgactctgttggtcttcctgtggagttcctatccccttcgtccccctccccaccctgcaatctttccctcaactcttctttaggcctggtggtggtggtgtggtggcatggaggaggtggtggagggggtgggggtctttaatcccggttcttgtgagaccgaagcagggacgatttcggagctctgtgagtttgaggccagcctggtctatagatctagttccaggacagtcagagctacatagagaaaccctgccccgagggggggggggcgcggggaatggttaaagattattgcaggacccagctgatctgtggaagaggtaacgggtgtttatgtttttcgaaactcattgaacaatgcacttcaattgtgcgcactttagaaatataaagccaccacgcgaaaagctgcgccccaacttaaaggcaatttccaaggtacttctgggtccttgcggttcagtggctgtctaggttcagaaacgaaactggatccccgccccgcccccccgccccccccctccccagcgccctgaggcagtttcgatttcctatggccagccggctaggcagctttttcatcgggactccttggaaagtccccacttcgttcatctctggcggatttgcgggagccagggcgctcatcgatcgcctggagccacagaatgacagcggagcagcggcagaatctgcaagcattcagagactatatcaagaagattctggaccccacctacatcctcagctacatgagttcctggctcgaggatggtgagtggtccccaactggggctctcaggctctccaccttagcgaggggaaaacatcactcagatcagaaacaattgaaggctctgcccccccccctcccccgcgctgtccttaagttaatttgtgtaacccggtgtatgtgagactcccaggccatattagagtagacagcatagggatttgatggtcaggaacaaaattcctgcaagctgtagtaacttgcataaggatgccactcttttctttctttcaatgctggggaaatagtttgtttctcttatttacaccttctagactgctgtgtgcctccctttgtcctgtcatgagaaactgagaaatcagaatgcgcccgcccctccttagattcctgtacagagcaaagagcaaggctttgggctcgggccaaaggtggaggtgggggccgcaggaagcaagaggactgactgacacgcacatttctgtcaaaggatgttgctcacaggaagtccgtggaagaaaactttctccagactccgtgtgttcagagtttaacacagttgttcatatctagctttggggatttgattggtggataatagactctttgtaaattgcactgggtgtttccacctgagcaaacagacctccccacctcacccccacccccagggagaagggagagggcgtttgaaggggtgaccgagggcgtgcggcagctacttttcattttgccagttaaagcctagatgtctttcctggcgttggacgacggtggcaactgcaggttaattctgactctcttgagttccgaagcctaacaggctatgcagagaggagtaaaagagcactacccagggctacccacatcccggttgtgttagagagaagcagcaaaaaagccctaatgattgggggcggggtctgaggagaggaaacccacccaagaggtttcttaacaccagggtcacttgcctttcaatcctttaatctgatctttagtcatttacattagcatacaaagtaactagtttcaatactgaaacaaagtaactagtttgttcagccattcctgccattgctctttgttcttatttaattgcctcttctgtggctcttccaccccctttacctgtccctctctggatgccctcccccccaaatggtaccccgttctgctttcttataacatgaggttcatcacactccctccctccctccctcccccatttaaagtatcatcctttcctctcagggtgcctgttttagtttcatgaattttagggttttggttttttgtctgtttagttatgagatttttttaaaaatgtggattatgttgaatttgtagattgttcttggtgctagaggcctttttatagtattatttccacccatcttgggagatctttctgaaatcttccagtgtcttcaagaatttttttttcccactgccttagaagtttgcattgtagctatcgttcacctctttggttagggtttgttgttatttgtttgtttgaggctattgtgaatagaactccctccttcccccatatctttctgggccaggttgttcttagtatgtaagtaagctactggtttctgtatgtttatttagaaccctgcctcttggttgactttttatgagggctgagagtttgtggtagtctttggggggtctttataggattatataagaatcatttgactcattcctttcctatttgtctaacttttgtttgtttgtttgtttgtttttttgagacagggtttctctgtatagccctggcagtcctggaactcactttgtagaccaggctggcctcgaactcagaaatctgcctgcctctgcctcccgagtgctgggattaaaggcgtgcaccaccacacccggccatcatttccaagttaaagatttgatctacattagacgccgccacgcagaaaaccttgagacttggtggaaaggccaaaggccattaaaataaattttcttttttctttcttccattctttcctttattccttccttcctttctttttgttttctttcttttctttttcctttttcctgagacagggtttctctgtatagccctggcatcctggaactcactctgtagaccaggctggcctcgaactcagaaatccaccagcctttgcctcccaagtgttgggattaaaggcattcgccaccactgcccaaatattttatttatttatttatttatttatttttatatatgtgatgagtacactggaaattccatcaaaaagagcaggtttgactggtgtcactagatttactattgatagggatccctaaaggagagctaaggtaaagggctctccctctcctaggtcttctgcataccttccttgagtgttctgggccagatctcctaagctctaagaatgtgctgaaaacacactgggaactggctccctccttgggaatttgtactccctctgctgtgggaaacttggatataagaggctacaggaggacagtgagttataccccaggcacagagttagcgtgtacattcaaaacgcataccattttgaaagtagcagctgctagcatttcctgtcacctggtcaacctggtctctttagctgccccaccccttccacttttctgctgtgtttcttttactctcttagcaaaaattggaatgaaagaccacaaatgtatttgtaattcaaaatgcttgctgcatcagctatactcgttactgttgccatagggcgttcattcccacccacccccaaccccttagtccagcagttgcttcagagttttgaagaagaggaggaagcctttcttcttccatgtgacaccctccactgcgacttctgcttactgtggggaacttgagtggaggacgggagtgtgcatagatgaaagagtggaggacgggagtgtgcatagatgaaggagtggaggacgggagtgtgcatacatgaaggagtggaggacgggagtgtgcatacatgaaggagtggaggacgggagtgtgcatacatgaaggagtggaggacgggagtgtgcatacatgaaggagtggaggatgggagtgtgcatacatgaaggagtggaggacgggagtgtgcatacatgaaggagtggaggacgggagtgtgcatacatgaaggagaggaggacgggagtgtgcatagatgaaggagaggaggacgggagtgtgcatagatgaaggagaggaggacgggagtgtgcatagatgaaggagaggaggacgggagtgtgcatagatgaaggagaggaggacgggagtgtgcatagatgaaggagtggaggacgggagtgtgcatacatgaaggagtggaggacgggagtgtgcatacatgaaggagtggaggacgggagtgtgcatacatgaaggagtggaggacgggagtgtgcatacatgaaggagtggaggacgggagtgtgcatacatgaaggagaggaggacgggagtgtgcatagatgaaggagaggaggacgggagtgtgcatagatgaaggagaggaggacgggagtgtgcatagatgaaggagaggaggacgggagtgtgcatacatgaaggaaaggaggacgggagtgtgcatagatgaaggagtggaggacgggagtgtgcatacatgaaggagtggaggacgggagtgtgcatacatgaaggagtggaggacgggagtgtgcatatgaaggagtggaggacgggagtgtgcatacatgaaggagtggaggacgggagtgtgcatagatgaaggagaggaggacgggagtgtgcatagatgaaggagaggaggacgggagagtgcatagatgaaggagtggaggacgggagtgtgcatacatgaaggagtggaggacgggagtgtgcatacatgaaggagtggaggacgggagtgtgcatagatgaaggagaggaggacgggagtgtgcatagatgaaggagaggaggacgggagagtgcatagatgaaggagtggaggacgggagtgtgcatagatgaaggagtggaggacgagagtgtgcatacatgaaggagtggaggacgggagtgtgcgaggatggatgagtggagtctgctgcctctcaaaggtcttctggttccatgagttgttatgactcccagacccacatgggaaggtctggtctgttatcttccagtgactagtgcttctgcaggctactcacttgcccttgcttctgtttgcagaggaggtgcagtacattcaggctgagaagaacaacaagggcccaatggaagctgcctcactcttcctccagtacctgttgaagctgcagtcagagggctggttccaggcctttttggatgccctgtaccatgcaggttggttccttcttcttcctcacagttcagagtacttcactctgctgcctcagaaggctgaggggagaaaagtgactcgttctgtgacatctgtgtgtggcttctgcctcaggcgggaaatgtaaagactattttgaatcagataagagaatggtttataccagaaatatccaaagcaatctacagagttgtaactactaggagaggtgacaatattagtagcatgccggtatctttcaagaggagaacgagtaaataaatcggttttataatgtttacagtgctccattatactgcaatgaagcgtgtggacatgtctgtaaatgacaacccagctgaactgtaggcacgcagcatttaaatttgaatatcataaactataatagctataaagttccacatgagtcaaactaaacatatagggaaggaaactggatcttgggcgaccctggctgaccagtcctggggagtaagcttaataaactctccctgtctgactgagatcggtgtcctgtggtttgtgaggcaattcctggactctaacacttaggcaattacatttcttgcccctctgccactctagcttattcactggtgaaagaaggagaatactttagtgttaccaacaatgggggggggggggcgggggatgggaaatgggaaaaagcaggcccggcccagtgtagtaagaaagaaacaccaaagaaagccaagggctcctgttgctttcattgtattggagtgtttgtcagtcggctgggggatggggtggggggtggggaagcacacctttaatcccagctctggggaggcagaggcaggcagatccctgtgagctccaccagtcatggctggcctcagcaagaactgtatccatcacatcctaacacaggtgtgttgaattaacatggtactgttaaagcaaacacgctgccttcctcgggtgctgcggtccctaggaagccacacattggcagcatgttggcagcagttgtataaaaactaatgcttttttttccttttctttttaattcggtaaaagggtttaaatgtcatttgttataaaacttggtttcctgctatttccaggattaacaattgacttattctttctatttcctgctttatagaccatcattttgatacattatctatttgcatctcagtgatacatgcttatcttacccttttatttcgttttaagaattgtgtgtgtgtgtgtgtgtgtgtgtgtgtgtgtgtgtgtgtgtgtgtgtgtgtgtctgagagtgggcatgcatttatgagtgcattgcctagaggtcagacattcccctggagctggagttaatggcagttgtgagggactgacgtgggtgctgggatctgaccccagtcccctgcaagaacacgatgaaccttacttgctaagccatctccccagcccttagctgttgcagttactctccattccaaataagccctggcaatgaaaacaagacttaattcatatgaatacatgctgtgcacctagattgggcagatctaccgctacactaccatcttctccatctatgagactcccccttttttttttctttttttcttttttgtggttttggttttttgagacagggtttctctgtatagccctggctgtcctggaactcactttgtagaccaggctggcctcgaactcagaaatccgcctgcctctgcctcccaagtgctgggaattaaggcgtgcgccaccaggcggtttctccaggctgtgtgcttctgctccacttttcttcctcctcctctgtggtatcctctccctcttcctctttctccttctctcttcccaccttcctctccaacttccctttatcagcccaatcaccagctctcctttattttactaattgaggtgggaagcaggtttacaggaaatcaccggagtgctgactcattccttgttcgcagccactcaatgcagaatggaattaccatcaaatataattagccccagggctatccacaacacttacctagcacatcaaatggcccagcaggggatcaagagaaaaggaaactcaacttctgcttattttcctcatctcttatgtagccccatcagagaagctgttgttttccttttgtgggctctaactaatttgaatattatatttaagatctattctcttaagtaaaaatggcacagctaactttaactgtaaaattatatgaggtttactaggaaaagtcttgagtttaagcaagaaagggaattttaaaacatttgtattggaacataagtgctggaacatctctctttgcaagtgaggtgctttgtgtgtacaaccctaagagtttcttttttttttttttttaatttatttacttcatatttcagacagatctcatttcaggtggttgtgagccaccatgtggttgctgggatttgaactcaggacctctggaagagcagtcagtgctcctctgcgctgagccatctctccagcccccctaagagtttcataaaggaatagtctgcattaataaattcagaaaaggctcagaatataaagccaatatcatcaagtaggtttccagtttatgtattaacaaataatgcaaaaaagattttaagcaagcgattccttttataacagcaccaagaacaataaagttaggaatgccagtgctcctaactgctgaaccacctcagcaatgcaaacttttacatcttagcactaagtccagctcctaattcgtgaatgtaagatgtcattcatgtccgtgtccctatggttcgtttcagaagtggtttatggtcttcgggtcataggtctttcccctgctcagctttgcttattcctaactttatttaaagttctcactgttgttataaaaggaatcacttggggctggcgagatggctcagtgggtaagagcacccgactgctcttccgaaggtcctgagttcaaatcccagcaaccacatggtggctcacaaccatccgtaacgagatctgactccctcttctggagtgtcggaagacagctacagtgtatttacatataataaataaataaataaataaatcttcaaaaaaattctaaaaaaatatggaaaaaaaaggaatcacttagttaaaaatctcattcctagccggttgtggtggcacatacctttaatcccagcacttgggtggcagaggcaggcggatttctaagtttgaggtctacaaagtgagttccaggtctctgaaaaccacaaaaaaaaaaaaaaaaaatagcactggctgctcttccgaaggttctgagttcaaatccctccaaccacatagtggctcacaaccatatgtaatgggatctgatgccctcttctggtgtgtctgaagacagtaacagtgtacttacatataataaataaataaatctttgggtgggagtgagcggggctggagagagaaggaaaagtatctgaagacaactacagtgtacttacatataatagataaataaatctttaaaaaaatcaataaatgaaagatgccaatattacccagagttggcacagtgatacctttcataatgccaaattttggtggcaggattgtttgtttattaaacaggaatagaaaaatttactctcaaatttgtatgaaatcttaaatggtcaaaatattggaaagagaaactcacttggaaaccttgggggacttatacttcctggtgtcaaaacagtacagaacctccataaagccagataattagaccattagcccagaagtaaactctgaaggatatggccaaaggttcttcaacaagggtaccatgaccacccaaaagggggaaaaaaaacccagtccctttaatatgaaaatacattggggtaagtgggtatacatatgggaatgagaggtatcaggcataatcttgtgctatgatgtgaatttgaaatgttctcatcacattcatgttttgagtgcttggtccccagcttgtggggttttaggaggtagagcctagctagccaaagtaacacgtgtatacgttcatgcatgtgtgcacacaggtatgagggtacttgtgtgtagcccagaggctgacattcagtgtcttccttaggagctctccaccgtatgtttttgggaatggatctctcattagacccagaatttaccctctcgggctcgactggctgggattatagggtcatgctgctacacttggctttttacatgatagctggggacaggaactcaggtcccagccttgtgtggtgagcacttttctactaagcaccttcttggtcctggagctattttgattgttttagttttttgggtctataggggggagaaaaaaaaaaaaaccacattgtcttcccagggccttgaatgaagtaaatgagggtctgagaggcaggcacgcctggtggatctgtccaaaaaccccagagtacggcattcttggattcttttagtcagaagtcattttccttctccatttgcccattgacttaatcttttcttggaatggtgtggaaggaaacacttttcaagggcaggatgtaagatttgtatttcctctggtcttctttactgtttcctcttgagaagataaacatgatgaatttgactaatttaaaagtaaattgagatgacaaagagatggctctgtgattaagagcacttgctgatcttgcagaggacccaggtttggttcctagacttacatggtagctcacaaccatctgtaacttcagttccaggaatctgaccctctcttctgctctccaaagataccagacacactcacgatacacagacacatgcaaagtaaaatagaaataaatattaaaaaaaaatatattgtgggttgttgttaaagtgcgtgaggggcattttgaagattttattctaaggtcaaatacaaggcctcatatctgtccttaggacttgaccctgaaagataatgaattttaggagacctaaactgttgggtaccaaaaatgagtattactcccattttggaaaatcatgaatagctgtattagttgactttaactactgtgagtaaatgcccgaggaaataaaaagcagaaaaatgagagccaagaggatctgtagcttctgcacccgtgcgtggtgaggcagggcaccatggcgggagcatgtgggaaaagccgcactttctggtagacaggtggcaaagggtgccgggcaggggccctggacaagatgcattcttcaaagcacatctccagtgactcacttctcccagagagggtacagttctcagttgctccttctgtatgaactcaatgtgctgccagtggtgacgccaggactcgaaggatgtggtcaccaggtcccgcagggtgtggtcacctctaaagactgtcaccagctggtgtccaagcctgcaacctgtcagcctcatggtctggctccccagactgtccagtgactgaggccatttgcagatggttttcagttcccttgccactgatttgaacaggattcccatgattttgacttcaaagcatttttatgttggatttgcttaagaaatccccatttctcttttctttttcaggttactgtggactttgtgaagccatcgaaagttgggactttcaaaaaattgaaaagttagaggaacacagattacttttaagacgtttagaaccagaatttaaggccacagttgatccaaatgatatcctttctgaactatccgaatgtttgattaatcaggaatgtgaagaaatcagacaggtaaaccaatgccaggtactaaatttgaagaaaaatgcagagacattggaaatgcccatttttctgtcttgttttaggcccaaggataattgaaacccataaaagctctcatctagcagatataatgactagaatagaatttttaaagtgaatggggtaatttttgtgctagactattagaaaattatttaacctatttgcagttaaagttgcccccttactttaaaaaaaatagtggtttatgcataatgcaaatcacaccaaacagtgcaacaattaaaaggaaaaatatgtcaggctcttgggcatagatacatttattacagtctcgcagtcacttaactagtgatgtgatgccaggcagttctctaagcatctgtggggtttttgttgttgttgttgttgttgtttgtttgtttgtttttcatgtctaaagtaagaaaatttatcttttgttttttgtttttttgtttttttagatttctttattttattttattatatgtgagtacactgtagctgtcttcagacactccagaagagggcgtcagatcttgttacggatggttgtgagccactatgtggttgctgggatttgaactcaggaccttcggaagaacagtcggtgctctcaacccctgagccatctctccagcccccttttgtttttgtttttgtttttgtttttgtttttttgtttgtttggtttttttgtcgttgttgttgtttgtttgttttgttttttcgagatagggtttctctgtgtagccctggctgtcctggaactcactctgtagaccaggctggccttgaactcagaaatccgcctgcctctgcctcccgagtactgggattaaaggcatgcaccaccacgcccgacgaaaatgtaccttattagcactcttttagggctaaatgagaggtcatgcacaaaatgtgtatgtcagcttgatgcatagcagtctatgcacaatgcatttcagttatcattagaaagaaaagtcatagaacatctgcttagaaaagagacctgctgctgtgctgttaggcatttccaaatggctctgtgtgccgatacatccttagggtgaatggttagcgtctgggttaacgcttttaccccaggattgctcttggtcagggatataaggattcagaagatgagaacatttgccttggcatattgataacacattataaaggacaaaggtgaagaaaggaatatcttaaaagctagtgctggacagggcaaaaagatgatgctaactaagccctactcaactatacttcacagtgatttcaatcagataccgcttccacaaaagcttgccagaggaaaggctgagctgcctgatcagtgtgctgcatttgtctcccccagatccgagacactaaagggagaatggcaggtgcggagaagatggccgaatgtcttatcagatccgacaaggaaaactggccaaaggtcttgcaacttgctttggagaaagacaacagcaagtttagtgaattgtggattgttgataaaggtggggtgctccaagaaagaaccctggaccctgctgcgctcctcccagttctccccactttactttccatcagaggcgctgttcacttcagataccaaaggctatatccctaggatacaagcagtggaaagctgaattctgggaggaagggaactacatggcatggaattaacccgaccaggtcaaagaatctagggaaggcttccagccccaatttgttatcagagaaatagcttgagaattctagacctaaaggttcaaactgcaagacttacctccctatcagagcagaggctgagtgttgggggtgatagctatggactggtgctcttgcccggaagccatctggactccgacagagcaagagtaaacgaagattttctgtgtttaagccaacctcatttggcttccggaaactcacttcttgctttaaacagaccttgataaatacctgagtttctagtttcctttctcacctagatttccttagaacataaattattccagaaactctctacatcgttggtcagagatggaatcctgtctctttagtgtgctcaggaatgacgcccctgcgttattggcgtgagttccggagtggggaggggctccggatgcaaactgctgagagccccgggttccacacttggagtcgcgtagttccagatgaaactggaattcaattgccaagttgagcttcaaactcagaataatccttgcagttgttttaagccgtcaaagtggggctctctagatggctcagtggataaggttcctgccactgatcctgaagacccaaattcaacgtccagggcctacatgatagaaccaatccccaaacagtgtcctcatccctcggcacactcactgtgtcgtgtgtgacacacacagtaaacaaatccatttcaaaaataaataaaatgttaagaaagtgcaagaccgtgattgtaagagctcaacggaaatttagatgtttagtgttagtgttaggactttttgggacttccccaaccaaaaccataatcacattgcgcatgcttttaatcccagcactcaggaggcagaggcaggtggatttctgagttcgaggccagcctggtctacagagtgagttccaggacagccagggctatacagagaaaccctgcctcgaccaccacccccttccaaaaaaaaaaaaaaaagattctaagctgtaagctgttatttgtgtttatgattgtttgcttgcctgtttatcacaaaggtttcaaaagggctgaaagcaaggctgatgaggatgatggagcggaggcgtccagcatccagattttcattcaggaagagccagagtgtcagaatctcagtcagaatcccgggcctccttcaggtaccaagcatcgtttgctctcatccatgatggtgtcccccagcactttgatgccctttgaaaaaaagtctttttaaaggatgattaagaaaagaaagaatttgtggggcaatagggacttcataattagaatccctgctcctgtcttccatggcctctgcatggccttcaaccctccccctcctctcccctcccctccccctccctccagtatgtatgccttcatctgtaccgtgttcccagaacttcagtgtccatgacttctcaaagcagccttgctctctaaagaacacttctgctcactaagcaatggctttgagaatctgggctgacagctggttttcctcggctgtttttgatgatctgttcttactttgttccaagtggctttgttttgaattaggccattcttgctgtcctttttcttgataaagtttccacgattaagaaagaattcatggggctggagagatagatggttcagcgtttaagagcaccgactgttctttcagagatcccgagttcaattcctagcaaccacatagtgactccagcgtctgggttaatgtttttaccccatctgtaatgggctctggtgtactcttctggtgtgtcagaggacagcgacaatgtgtatattcatatacattaaataataaataaatcttcaaagagaaaagaaggaaggaagaagtaacagagagagagagagagagagagagagagagagagagagagagagagagagagagaacacactttggccaagatcccaaacctcaaacaggggcattgttgctagagtcagaactcatgtccactgaatggcagttgcaccatgattccttgtagcatgaacccttcgataactttgtcccctctatattacagaagcgtcttctaataatttacacagcccattgaaaccaagaaattaccaactggagcttgccctgcctgccaagaaagggaaaaatacaataatatgtgcccctactggtaagtcagttgctgtcactcacagaactctctggcttcgctttttcttccccctttgggggctgtaaaaggaggagttttccccgtggcccatgctgcccatgggagagctggtctagcagcttaaggaacctggacagcgataaggagggagataagtgtcttctttagtttgcttttggttcttgctacctgagtgcacgttacttaggaagtagcttggcacttttcagccattgtttaaactgtcattgttagtgcggaggagggattattagtttatttgtatcccagtggtcatagagaagccaaaataagtaccattctggaaaaacagctaacacaggttatctgttggtttttttttcttttctttttttttcttttttcttccctactaaaaggttgtggaaaaacctttgtgtcgcttcttatatgtgaacaccatcttaaaaaattcccatgtggacaaaaagggaaagtggtcttcttcgctaaccaaattcctgtctatgagcagcaggcaactgtgttctcacgatattttgaaagacttgggtatgtactactacaatcaatctaactgctttgatttttggtttttgttttgtttatgtttgtattttaaattctagcccctttggctggttttgggggctttgttgtgtctggttttgggggctttgttgtgtctggttttgggggctttgttgtgtctggttttgggggctttgttgtgtgttttctgagacagtgtatcacgtagccttgagttgtctccaacccactgtgtagctgaggttggcctagaagagatgatcttcttgcttctacaagtgctgagattacagtgtgcactggcatgcctggctgttctctgattttctttcttttttttttttttaagatttatttatttattatatgtaagtacactgtagctgtcttcagacactccagaagagggcatcagatctcattatgggtggttgtgagccaccatgtggttgctgggatttgaacttcggaccttcggaagagcagtcaggtgctcttacccactgagccatctcaccagccctctctgattttcaaagctatgattaaaggaaaatcgccatggacttaacttttagaggtagttcctttgtgcaataacatttttggtttaactttaccagaaatgctaagccctcatgtcatgctctgacagttaatgaacttggtggccaaatttaacatgtaggcgatacacaggtcatccttaatgatgttatacttgattggctattactcttttcaaaatcatttctctcttaatgacttgaaagaataaatacactgtgatcagctataacctcttgcatttcctgactccccggctttgtgtcaggcctgtgagaaagttcaaggtactacccagttgtactcttttgggcttgggctgacttctttaattgctgctctgacctagacttctactttgtctccttgttcattcacatcaaggttgatgataagggatttctgtcattccccaggtacaacattgcgagcatttctggggcaacatctgatagcgtctcagtgcagcacatcattgaagacaatgatatcatcatcctgacaccccagattcttgtgaacaatctcaacaacggagccatcccctcgttgtctgtcttcactctgatgatatttgatgagtgtcataacactagcaaaaaccacccatacaatcagatcatgttcagatacctagaccacaaacttggagagtcacgggacccactgcctcaggtatttccaatcttctaagaagaaccacagtttttcagagtcccacttagttgctcttttgtagccacatttgagcttgccctcctcggggtctcagtccatcggtacaactcagtggtcaatgttggttcattcatttgaccaacagttgtcccttggtgtccagggtagatgcccttcacaaaaaacaaaatctaggctgcttaagtctcttgtatgagatgacattgcatttatatataatctacacactttcctcttgcatactttaaatcttctctagattactgatattgtacagtatgatgaaaattttatataaatagttctagtactgtaatttttagggaataaaggtgggaaattcatacatgtttagtacttatgaagttttaaaaaatatttttgatccatggttgtatgaattcacatttatataacctctggatttggagggccagctgtataaaccatgggcttccatggaccttgtgcattgttctaggctctgggacaccaatacagaagatatagtcctggctctcatacagttaagtttgcagggagccaggaacatagtagtcacagcttatcatgaggtatgctgcagagacaggtaaagggtgttgtcagaacataaggggtaataaggcatagaaatgaagggaactgacaggggcttgccaggtagatagcttctggtttctagtaagagggtgctgtgtgtccaggggctcagggaaatggaagggcctgacatgccccagaaccttaaaactctacagtatcacttgagggtagagtgtgaagcagggaacaacagtcaagctgattgttatgacagatgacccagagacaccaggagggcagagcgtcagtgggcaagtggatggcttagcacagggaacaagcagcagccttctgatgtcatatgagaagagtcacttcagagtcattcttacatgtgacaagaggagtacaaattctccttctgtctatcataggagagggggtgctttgctgaaagtcaacgatatagaaacaggagggggctagagaggatgaggacggtttgactcaggcactgatagatgcaataaagaatgacggtagtgtatctatcaggggccccaagaagctgtaaaccatgaattatatacaattcttttgctccaacaataacctttttaggacgtgcaggttaaaggacatttagtacaggacccacagtttgttattctcgagtatcgttgctaggaagcagatttcttaccgtccagctaatcatttaggtgaatgcttactgaagggtgttatcatactgaatctacacagctctcttgtacacgactcactgattgttgaaggtatttgtccaggcgcacaaaatgcatgtgatatgaatgagcctggaatggactttttcttcccattgtgatgtttagtaagagactgggggataaaaaaaacagggtagccctgcctggaaaggtttcctctctgttctggatgacacgctagatttatttccgagctttgctccaggggggtctttgtgctggagaatgtcagagagccagtggtggggtgctccttacaggtcgttgggctgactgcctccgtcggcgttggagatgctaagaccgcggaggaagccatgcaacatatctgtaaactctgtgccgccctggatgcctccgtgattgccacagtcagagacaacgttgcagaactggaacaggtcgtttataagccccagaaaagtaagtggaggtcagcagcccacacctcgcgactttgtaaccttctgtcccctctgcgtcagagacagtggatgaagtttgatgctgtatttgtttggtaaaagcatagtggttacattgcctatctttctccctagtcaacctcttctccctagcgacgcatgagtctcaaaggtagccagaaagggacaaacatccctactctttaccagcagctgagtgaaggaggcagtgggaagattcaagcattttgaaagcctcaatagctagtggcggaatcaggtctctgtgctccgggccctaggcaggggctatgtggccatcttgttcttgtatgtatctgatcattgtagtggcatgacccgaatcatgacagttcaaaaggccagaacatgtttttaaaatgagcttcattagaagatggttattacttattaactacctgtgtaagcagggaggtaccgtagttacccacggctggatcttggcctgagcactcgttctgtgagttgacagcaggatcaatggcagggtcaatggcaggatgagcaatgggggggtgggggttgggatggcacaaccctggttctttctgagagtcccccgtggagagtgtgaagaaggtgcctccccacccacgcccaccccttagcaacactcaagggtttttctacagtttgagcccttggagcttagtctacttcaaagtcattttgtgtcactttctccgtctatgcaaaccctctacgagctattctgagggtgtgtcccagctcctgcgcgccttcctttttcccttattattcatcttgcggcagcttccccggagagaatgaggtttcctcccctctttgagagatgccttcctggcctgcacctgcttcccagggctctgatgggcgggtttaggagcacacctttgtttcctttaaggagtgggtgggttggggagcagggggaggggggagggaggggatagggggtttttggaggggaaaccaggaaagggaataacatttgaaatgtaaataacgaaaatatctttaaagaaaagaaaagaaaaggaaagaaagaaagaacctgccttctgtgcagcatagggtagctcttgtcagctctctgtcactgaaacaggacatgtgacaggcagttctttttctgccaaaagtacacaaatgtgaacgataagctcaatgggggcactcttgggggctcggaggtgcgcaggagaataggaaatcaggaaaacggggctggagtatggtatttgccgaaaccagaaggctgccagacctgccacagtagaggcaccaggaaagctgactgagacgctggcttagactagaccaggagagacactagaatcagaagcagttccgaggtcagaggcttctgaccgcctgctgtgatttgggccacgtgagcttggagcctgtggctttaaaggacttacccaggatggagcagcttcgggaaatggctgcataggacctgggtttccttcagcttactcacatgcctttgaccccagtttccaggaaagtggcatcccggacttcgaacacgtttaaatgcatcatctctcagctgatgaaggagacagagaagctagccaaggatgtctccgaggaacttggtaagcctgtgccaagtcctggagagagaaatctcatgtttcctgtcccttccatttagaggtactcatggattgctcgttagtgtcttcagttttgggtgagattatactcagaggtggactgacttatttattcacacatatttctttctgtctctgtatcttctttatctcttcattcttttttgccatcatttttttctccattcctttttaaaaagatttatttttatttgatatgtgtagatgtttttgtctgcatgtatgtatgtatatcacatgtatcagatacccgggaactggagttacagacagttgtgagctaccacatctgctgggaatcgaacccatgtcctctagaagggtagctggtgcgcataaccactgaggagcccccatttctctagctgtttttaagacaaggtttttttccctgtgtccctggttggcctgaaacttgctatgaagacaaggctggctttgaacttgcaggggtccccttgcttcagcttctgagtcctggggtctctggcaagcgccaccatacctggctcagatatagactttcttaatcctaggttgtttaggaaccttataggagttctttaattctctcttgcctttttctttttaaatacaaaacacatccacctggacatacatacctgagaaatactgtctttaaatcatcttctaaatttcctttcttcctttttttcccctcttgagatagaatctctgtgttcagtgtaggctggccttgaactgggaactctgcccctcctcctcctcctcctcctcccaagatgtgcatcatcactgagctgccattagagtgccattgtcccttccaagagcagttcccccagtgacctaacactctctcactgtcctcagctcttgaaagtgtcaccacctcctaacctcacacactgaggaccaaccagccttttgccacatgagcatccagaaggcacttagacagtagctaaggcacagcactgggggaggagtttgaataatgaatccactatgggtcctaaagtagtagggtagcaagcatgctctctcctctagagttttggaaactctctgtaaggtaaagagtaaagagaccaggtagtcagtacatggctcacctaggaacaagataacatggtctgactaaagtggtggatggacagacggacaggaatagagttgtatgacttacttttttgttttgtttttgtttttaaaacagtctgtctgtatagctctgactgtcctggaactctctttgctggcctcaaactcacagagatctgcctgcctctgcctccggaggcattcacactttagaatcttttcccacctcctcacattgagtatctgtcaatagctgcctcacttcttctggaacttggacgtttttcattgtgaactgggtgtggtggcaccatctctactcccagcagctgggagcctgagtctgaggccagcttaggctacatactgagctcctgtccttggggcggaggctgggaagaacttgtcactgtttcttgttggtacccgtcctgtgttctgttattgcaaatgtgagggaagccatttaacacacaaatgcatttcacttctttgaactgtactgtgcttgtctcaagaagcccaggacacaaaacaatagagcaagcatctggggctgttcccacttcgcctttccccccctacccacaccaatcttcccctgagtctgaatcgctgtgaatcccacagtagaaccaagcagtcaagacatgcacatgcgcacacagatgcttccgggataactgtgtttgactccgccttgtggttggtgctgcaagtgctgctctgagatcaggtgtttgggcttcatagcaacatagagcatgctgggaagggtcctggtgctcccatttttatataactgtctccgatgaagctcttgagacgtgctactctaatggtatcttcattttgaaaggcaaagtgtgtccctccttctcttcctcctcctccttcttccttacccctcttcttcgttctctgttatttctgaactactttggctgtcagccccttaagcctgcagagcatagacaccacagagctaggcttgaattcttgcctcacccacacaatatgagctttatgacattgggggtaaattagttttccttttatagaagatttatttacttaaaaaaaaattatgtgcatgtgcatgtgcgggatggtgttgttgcctccaggggtcagaagagggcgctgaatgccctggaactggatttacaggtcgttggaagccacccaatgtgagtgcagggaactgaacttgggtcctctacaagggcctaactattgagccaccacttctgctccttactcaatctttctgaatctgtttcctcttttttttttttaaagatttatttatttattatatgtaagtacactgtagctaagctatcttcagacactccagaagagggagtcggatcttgttacggatggttgtgaaccaccatgtggttgctgggacttgaactcaggacctttggaagaacagtcggtgttcttatccactgagccatctctccagcctgtttcctctttaaaaaaaaaaattaaataatgacctcatgaaattagaaaatttcaatgcaattatgaagcttgattttgggtcatttagtaaatagttattttacacactcctcccccccaccccccgcgcacgcacacaggcacacacacacacacacacacacacacacacacacacacacatagcttaagacccagtctacttcaggataaacatctttcttataatgaataagaaagaaaatcagaggaccggtgcttgcaaatcttttatttatctatttatgttcttaccctgtaggaaagctttttcaaattcaaaacagagaattcggcacccagaaatatgaacagtggattgtcggcgtccacaaagcgtgctcagtgtttcagatggcagacaaagaggaggagagccgggtctgcaaagcgctcttcctgtacacatcacatttgcgggtacattgctgctctccagggcttattctcatcaccgcgcctcctgggatctgtactgaggcagctgagagaacatcagcgtctcaagtctaagagcttagtgaggaacttttcccgaaagtcatcactaaccttatttgttttctgaaacttatcatcaagtctccaaaaactggattaaaggctcagagtctatgccacacctccctccagcttgtgactggtgaccaccatctaactgagctcaaaaaagtggctcctgtggccatatcctgaagctttcgtggtcttaattttgttataaagtcatatattagaatctcaggggctctggttaacacagagggaaggagtaactgtaagagccctcagctctgtttgctatgctctgggaactatttaaagacttactccacaccatgggattgtgggatctaacgcttaatggactttcagcataggtggtaagggccatcgttatgcaaggcccatgtacactttaagtatgacttggaatttaaggggaatgtcaaagctaacttgcttttgttattgtttctcaagatatgctgtttctcctctcccaaggtggagttttataatccaaagtgaatctacttttaattttctagctgagccaaaaatagaagccagcttttggttcagaggtttttattgtagacccactaagggccattcgccattaaaccctcagctgtactgtatgagaaagattttctgcaaaccagttttgtgctaaatacagcgagttgaacttgagtgtagtgaccatatgcgacctcagaaatgtattgagaatcacttttcatttcaaacagaaatacaacgatgcactcatcatcagtgaggatgcacagatgacagacgctctaaattacctcaaagccttcttccacgatgtccgagaagcagcattcgatgagaccgagcgagagcttactcggaggtttgaaggtgagggagatttctgaagtcaggagtccctggggtctggtggcttttgtggcagtgtgcacatcgtagttagcatacgtagccatcatgttgggtttaaggtgagatttgtaggggctgtgacggagcatgaccttagcatggctgaaatccccagcactaaaaaacgaacctatgctgaaactttagagccaaccaaccgacaacaggagggtttggcttcagagaaatctaatgcctgtggatggatctgatgcttgccccacttttcacttgggaaaatgggaaacagtgggatttggaaagggtgcttcctctaggtggtaggtagtgctattctgattaactcagtaattcagaaggtttaataacaacagctcgtgtctgatggtgtcaagattgtgctgtatgtatgtccttccttccttccttccttccttccttccttccttccttccttccttccttccttccttcctccctccctccctccctccctcccccctttctcttctcttctcttctcttctcttctcttctcttctcttctcttctcttctcttctcttctcttctcttctcttctccttctcttcttcctcctccttcatcttcttctttcttcctcctcttccttctccttcttcttcaaaacacagtctgtatagccccggctgtcctgaaactcactttgtacaccaggctggcctcgaactcagaaatctgcctgcctctgcctcctgagtgctgggattaaaggcgtgtgccaccatgcccagtttgtgcacatatatgtgcatatgtttattataaattttactgatacaggagatggcatagcacaaaacacacaaataataaacacggagttcatgttccacagaatgcctttggaggtcttttcagtacccttgtgtccagagccaaccagagacagcaattacccaatatggagttctgaaatgaaagtcagttttatttcctgttaatggcagaaataagaacaaaacgaaacagcagaagcattttggaagcttgcttgtttctcagtgatgggagcaacatttttctgagccagataatagtttttcaaacacgggtgggacatttctgcatttttacgtgatgcataaacagtagctaaatttaatccccattatatacttagcactttacaaagtctagccagacaataaaggatgaagcaagtgctatcttcatttccatggtatgggtacttctaggatcaccaatctccaaccatcaccatgttgctgaacttgtgtaaaattgagcagtaacacacactgacatttctaccattcatacactacaggtaagtacacactcaagagcgtagataatagtaaactgtaataaaatgagttaggaaattaataagcgtggctatttgttacatttgtttttagtcattgagctgcaagcataaagagttgaaattttaataatagttatatttaaaaccaggtccacaagtctgaagaacttaataactgaccataatctggtttgatctggttctatctagtacaccaccagtgtgtgtgtgcgtgtgtgctcctatgcatacttatacattaaaaaaaaaaaagatatcctatgcttcaatttttaacataaaataaccttctgacagctgggtggtggtggtggtgcatgtctttaattgcagcactcaggaggcaggggcaggtgggtctctgggttcaaggccagtctggtttacagagccagttctaggacagccagggctacacagagaaaccttgtttcaagacaaaacgaaacaaaacaaccacaaataaaaaatatatctttttgatgtttccaaatcagcaggtgtatataactctttaactttaatagtaacagtgtatttacctcagtttggtagcctgggatccattgagctgtttctcactaagcagtgttttggttgttggtttttttttttttttttttttttttttgtatttagttcatagtttcaacactgattgtccttggaatcttcttcagagtttttttttttttttttttttaaagatttatttatttattattttatttaagtacactttagctgacttcagacacaccagaagagggtgtcagatctcattacggatgttagtgagccaccatgtggttgctgggatttgaactctggaccttcggaagaacagtcggtgctcttaaccactgagccatctctccagccccagagttttctaaatagaactatgagtcaattcctatctgtggattgctgtatcaaagaacatgtgagttttgtattgctgcgctgcttttctaaaggggattcctgatgaaacgagtgtttactgctctgatctctggtgaacagtggaaaggttaaccgaaatagaaggccactgtttgttctaaagctttaacatttgtaagccttttgcaaaatgctctctatttgcagaaaaactagaggaattagaaaaagtttccagggatcccagcaatgagaatcctaaactaagagacctctacttggtcttacaagaagagtaccacttaaagccagagaccaagaccattctcttcgtgaagaccagagcactcgtggatgtaagtgtgtgtgtttacagattagctctagtttattgaaaaggttgcccgttcttcactgccttataatcaagtatccatacatgtgtggacctgttctgatgatttgttcttacaccaattgtcattgtttgtattgacccacagttataagtcttggtcctatagaggaagccctgcatcctttttaaaaatttaaaatttccacttccagtcatcctgtaggttttgattaatgactaatgtgtcttatatacctcacagttatcttcatatcatcttttaaaaataatttactcagattttaaaaaccagttttaaaattgggcaatgggctggagatacagctcaatggtcaaatgcttgttcagcatgcatgactttaatccccagttctggaaaaagatagatatctctctgtttatgtagaatgccttgagtctggccacagcgcctccctctgtttatgtagaatgcctgcattgtttctgctgagtagtagattacccatagagccagaggcagaaaaagtcaagctttattattttatgagatccgtggatcaggatctggaaaggactggatacttatgcctcaaggtctcctgaggccacagtcagctcggcactcaaggctgacctctcggctccttttgcaggttgttggcaggcttgtgaagatgagcctccaacatggcagctgcccctgcctacagtgagatgagagactgaggagaagagggcctagtagacagacactgccattttataaagtccatcttggacctgatgtcccaccacatctcccatatttcagagataaactacagattatttttagaatataggatgtagaagtcattaagggtcgcttgtcatgtgatctttgctgtcttcttttgttaatgaatgtgggtgtttaccatgtgcgtgtcgtgcccacagagtccaggaagggggcatgacatgccctggaactggagttaacagacagttgtaagttgccatgtaggtgctgggaattgaactcaggtcctctggaagaacaaccagtgctcttaaccaccgagccatctctccagccccctttgctgtgttttattagcattttgtcatttttagtatagaggtcctgcatacattttgttagattcatacctaggtattaattttagtgttgtcattccgaaattgtactttcaaatatttctcactgtgggctaggaagacatctcagtaaagtgtctaaagtacaggcatcaggacctggcttccagcaacctggtaaaaaagccgagtacagtagagtactcttgtaatcccagccctggggacagagataagcacaaccctaactggcaatgcccaggtcccagggagttactcattactcagtcttaggcagaacgaaggtgggtggctgttaagaaatgatacctagggctggtgagatggctcagtgggtaagagcacctgactgctcttccgaaggtccagagttcaaatcccagcaaccacatggtggctcacaaccatccgtaacgagatctgactccctcttctggtgtgtctgaagacagctacagtgtacttacatataataaataaataaatctttaaaaaaaaaaaaaaaaaaaaagaaatgatacctgaggttgacctccacatgcatgtacacacacacacacacacacacatgcgtgcgtggacatactcccctccaacacagtcagccatgtacacctccacacaacacacagttcttccaattgcagctgtctgctgatatttactgtgtaattaatttacatggattgatctttcaccttaaagccttgctaaatttcacttactctatgtctgaagcttgtctttttaatcacttaaaatatctcctacattaagccataatgaggcagagttctatatcactagcatcaattgttgtttggaatttaggatttgccagtctgaaatccatttttatctttagttgtattctctttttgcatatacatccattatatcaaattgatgtgaggtttaaagtttacaagtggtgtctaactggccgttgcttttcacttttaggctctgaagaaatggattgaagaaaatcctgcactaagctttctaaagcctggcatactgactgggcgtggcagaacaaaccgggcaacaggtatttatgtctattgaattagatttagtatactatgtatataaaatgtataaacactacattgttttagtgtttctatcagtcagagctcaaccagagaaataaagctactagattatgcatatgtatgtggtatatatgtaagtatgtatgcttatttgtttgtttgtttatttatgtacttagagatagggtttctctgtgtaacccttgctctcctggaactcactctgtggaccaggctggccttgaattcagaaatccgcctgcctctgcctcccgagtgctgggattaaaggtgtgcgccaccactgcctggcctatactgttattctttacccagtagattttttttccccatctactgcctctttaatagttttaaaaaaacagtccaggcaatcctgaactctagctagtgtggtctagggaggaaggttatcatttcccataagaaccctatgtggctagctctcatcacagctacgtcccaagtcatatctcacgactgtatgacctgccctcgctgttcttcctgccagtgttgtttacactaaacaagtctccaccccttctctctccgtcctcaggcattgctcttgtacttttccattgtggaatttcccgatctcataaacatagaatggactcaagtgttgaatgtgtggtttcgagtctaacactaccctaatgtggctggattttcaaagttctttgccatctctccaacatgaatccaacttgattttcaagctttgctactgacatataaatcgagccttgaataatattttgtgtgctcatccatgcatgcatggatccatggatccaatcatgtgtccaaccactcatccacccatccgtgcatgcatccatctttccgtcatgcatttagaccttactcagctcctgcttctgtgaagaagcagccatccctgtcatctaacacccgaggtgcccctccccccgccacgttcccatcttacagatctcaccccacttcctccacaatggcttcctgctcatcatccttatagataaagatggaatctttaagcgtcatatttctacctgctcagccataacccataattgctgaccgagtgttggatggatgaatgaattggttaggatgattctgctattgttgttttctggatgattctttcttgttttatagctaacctgggaaaaaaggtggacttttacaaaaagccacaggttgcttggtgtttggacattttcagattcccttatctgtagcatttttacttcctactttgagacacatgttgtaatgtttatgccttactatcttcatctgtcaaatggaacaataaatagttgtccccagctcataggttaacgagaatggtgaaacctgagctttttttttttttttttttttttttgtgaaaatgactttggcacttttagatggttcaaaattagtagccagtttatgagtgagttgtacagtgacctctttatccaacacaggaatgacgctcccggcacagaagtgtgtgctggaggcattcagagccagcggagataacaatattctgattgctacctcggtcgctgatgaaggcattgacattgctgagtgcaatctcgtcattctctatgagtacgtgggcaacgtcatcaagatgatccaaaccagaggtgagagcggctgatgtcattcccgccccgcacccgcttttctcctttcctcagctgtaccatgtgattgacagcacagctgactctggtactcgaaatctaaaagctgactgccttggtcaggattgggtggttataggtttacccataatactccattgcaactctccacaatggtactgcaattttacccagcgttcaatggcatagtcgtgaaaatatcatatccactaggccagaggctttgccagtcggcaagtagacctttgatgggtgtggtgagtagctctctgtactccagagtctggtcccacctgaaccagagtctgacttcctttccctttcttgtttccccaagaacagcccccacattccctttccggaataacgtctctgtgcctgtcactcatcagtcacatccatttttcgtcctcctccaccgcttactgtgcggttcagccagccagactccgcttcctgctcgtccagtttctcagatactgtcgctctacatgttaggtcctatctctgtctctgccacacacaacctaattcttctacctagaacaagcactcctttaaatgcccactaccgtttatatctgtctctgcaagagcatgacaattgcattcctttctccgcattgcagaagggtcaggtgcgcgtgcacggtgccactgctgcgggctcatgccagattatctgtaaattagtgttgctggcagtgcagagcaatcagactatgccattggagaccccatgaaaactgccagagatggcttatctgtgtgctgagcacactggctagaacctgcatttgagtctactcttcggttcagcttccctagaaagtaggatgcagtgaatcaaagttgaactcgagaaatactcctcacatctctttccagtaacctcagagtttgacattaacacacaaagaaaacgttttctgcaggccgaggaagagcacgagatagcaagtgcttcctcctgaccagcagcgctgacgtgattgaaaaagaaaaggcgaacatgatcaaggaaaaaataatgaatgaatccatcttaagactgcagacatgggatgaaatgaaatttggaaagacggtaagtctctttttctgtgctactcttatggaatctgactagaaataacaaatgaccatggttggtcctgagtgtgtgtgtgtgtgtgtgtgtggtatgtgtttgtggccatgtgcatttatttatctttgtgtgctagttttggccattcaaataacctttctgttcgcatgtaggttcaccgcatacaggtgaatgaaaaactcctcagagacagtcagcacaaaccacaacctgttcctgacaaagaaaacaagaaactgctgtgtggaaagtgcaagaattttgcgtgctacacagctgacattcgagtggttgaggtgagtggccctggtgatttagcaccggttaaatcttaccatcttccggagaaatggttgtagcaagaacactatgttgtggggtttcgagtgttgaccatggtcctgtattaagaaataaaatcctgctaggtggtggtggcacacgcctttaatcccagtacttgggaggcagaggcaggcggatttctgagttcgaggccagcctggtctacagggtgagttccaggacagccagggctacacagagaaaccctgtcttggaaaaaccaaaaaaaaaaaaaaaaaaaagaaataaaatcctgcttctatgtgggaaccagaaaggctgatgttatttaagtccaaaacagaaaatggtgcttaacggcgagaagaggaggggggtctaattgtagctgccccagacagtcaggcaggatggataaggtttcccgttccactgcacagcagggtgaatactgcttatagtttctgattcattacaactcttacaaagaattagacgagaggaattcatagcttcagacataaagagatgaaaactgtccaggcagaagggaatgctaattactctggcgagatcattagatacttagaaattatcacactgcacaccctaagtgaggacaactgtgtgctttgaaagacagtctcactcagaccaggctggcttcaacttgagattcttctgtgactcagcctccccagtgcagggactctaggcatgcaccaccactctctccaaagagatagtttttcccagtgcagggatagaaaatgaaggctctgctagatacagtgttatgtccttggttagttccagggaggaggaagggctaggcataaaaatctgtcattgatttcttagttttaacaaatgtgcaactgcattcaaaatggaactgggctaaaggcatttgcaaatcttctgaggcatctctgtaactttactgtatgtcaaagattatgccaaagaaatgttaaggctctgattttgaagtgtacatggttctagtataaacctgccagcaaatgaatggtaaagtgggaaaatactatgaatatgaaattataaagatgcttttgttatggctacatacaacatgagcagtgatatctttgtcatgaccaatgtgggtccacctttcctaaaggggaaaaaaggctaatatataaaaatgacatattctgctagtgaattctctcttcctgttttgttttctaaacttccttattggaacagagaatgcttttataatgaaaacaaaacacctcattttaaaaaatataacacttgtaggttagctttctactttttcaccccttaaactttttttttttttttttgagacaggatctctctctatagcactgggtatcttgaaactcattatatggagattcccttgtccctgcctcccaaacgctgggacgtaaaggcatgtaccattacacaaggtcttcttaaaacttttaactaaggcaaaaaacctccagagacgaatcttgcagtcatcattcctgctaccgatggcgggggcagatggctgtgctagctaggggaggggtacagtccttattatgagtgacatccactttctgagtctgattctttagatgcagaaggtcctttcagctccaaattcaaggcttctccttcccagtggcctggagaacgagatgcttggtctgcgcttgcgtctgtagggtacactcttttttttttaattggatattttctttatttacataaagaacaccttcagttcccctacactggggcatctaagccttcataggaccaaggacctctcttcccattgatgcatgacaaggccatcctctgcaacgcacgcagctggagccatgtgtactcctttgtggatggcttagtcgctgggagctcttgggggactggttggttgatattgtttttctccctgtggggttccttcagttgtagggtactctcttaagtcaagtctaagtggggtctgtggacagcatggctcctgagcctgttcacacacataccctgtgacccctgggtgtcaccagccctgtgctttggtgccttcctggcctctggtgaacttgaactttgtatgtgaactcctctttgcttctgagttggaaagctgggtttcctcctttctcaggtgccagacgcccaggaaatgggtcctaatcggcctggggaagactgtctatatagtttttttttcttccaacttgtaaaataaatgggatcccactcattcctgacttttagctactgagtggcttctaagtcattttcagacccgtttctaacattgtgcgctccatctcttcctcctagacgtcccactacactgtccttggagacgcttttaaggagcgctttgtgtgtaagccacaccctaaaccaaagatctatgacaattttgagaagaaagcaaagatattctgcgccaaacagaactgtagccacgactggggaatttttgtgagatacaagacgttcgagattccagtcataaaaattgaaagtttcgtcgtggaagatattgtgagcggagttcagaaccggcactcaaagtggaaggactttcattttgaaaggatacagttcgatcctgcagaaatgtccgtatgacctcaggcttctccgtctcgtgccgcagggagccgtgccttaagcatggagttgatgagccaatgctttcttacccaagcttgcacaatcctttcttacacaagcctgcactgtgttgaatgccagataacctgactggttggtttcaagctggtgctgtccacacaaagcacacacgcctgaactgcggcgccgaatagtttcttcaccaataactcatagcgtagcccttggccatggtggggaggggttaaacttgtcccttttacacttttcagaactgcccgacagggaacgtgcagccactcggtacaccgagacgcatgatggctggcgtgctggaagggttcccgttctctgtctgctcgatctgctgtaagctgcctttgcccttaatgacagtgcccttaagaacagtgacttagttctttttcaggccaccagactgactgccagatcccttctgtcccttctgtcccttgcactgattcctttccggatttgaccctgccaccctgtcaccctctgcagagtctcctggtttctgtctcttccttggtttctttgctgactcaaatttggtagttgcaaggttcagtatgcacacatatatatttaaaatgacatataatttaaaatgtaaaagactatagttgacagctatgcttactgagatggtatttctgttctgttcattactatacatcttacccttgctctcatctgttcttttaacttgggccatttcccgtctttgaatagacatctcaaaccctgtctgtatgtctgtctgtttcccacctgtttgagacagggtctctctgtagaccagaaactccatatgtagactatgctggccttgaactcacagatccccctgcctctcaagtgttgaaattaaaatctttcaccatgcctggctctagctattttcaataaaggctcatgtttaaagtttgaactacttccaattcattccctgacgtggcttgttgttgttgtttacttttggagacactgttcctctctgtagccctggctgtgtagaccaggctgcccttggtcacagagatctgccagcttctgcctccggagcactgagattgaaggcctgcatcaccatgcctggcttgccctttcttcttaaacattatatattcaaatggcatttccgtgtttcttctcaaggtgtgccagtgcttcagagagcttagtttggggttcttcagatcaagagacaagtgtctgagcgctgttactgccaacagagcaaagtactcttcagttcagggaaggaacagtgctggttttgtaggcagtacagtggttttaacaccttcctagaacttacttgtaattcatcagttgtagaccatcaatggcctaaaccaaactgcagagatcatctgaccacataactccccttccaggacatttacatttgaagactatcccaagcccacccagagcacagtgggttacccaaactccccaggtcaaccctggaggtcaacagtatgacatgggatagcacaccacttctcacagatgcctagagaaattacccagcaacataactctttggggaaaaaacacctatagggattaggcttttaattgatagaataggtagaaaaaaagatatgtagtagttcttgatagtggttactggtaaaattcttagtgcaataaaatgaatttgccgagagctgactttcttttttttcttgtttttttgagacagggtttctctgtatagctttggctgtcctggaatgcactctgtagtccaggctggccttcgaactcagaaatccgccttcctctgcctcccaagtgttgggattaaaggcgtgcgccaccacgcctggctgagagctgactttcatttatgtcttttagtctatgttgcctttctttgctgctacagtttaagaactctacagcttgtataagatacctactggaaattatttgagaaaaaaaacttgtaaacattacaataatttaattaattaaaaatttatgtattttatgtatatgggtgtttttttctgcaagtctgtgtgcacattagaagagggcattggatcctctctattgttatagttttatgctgctgctgttgttgctgtgtgtgtgtgtgtgtgtgtgtgctatgaattgaactcaggacctctggaagagcagtcagtgctcttaactgctgagctatctctccagtcctggcaatgataaatcagttgaagtgaaatagtcctccccccccttttttttttttgccaatggggaaaagcagactaaatctgagaccaaatgaagttttgagttgtacactgacttaagccactgccaagcataccctggaatggagcaaaccctgggttactaagtactgaatgaatacaacaggaaggttttgagagatgggaaaatgcttgtctttggacttccctgatggaagttgcatctggactctcccatgagcacatcaccagtccccactagagtcctcacaggttgccatccatgtgtcctttttgaggctgagatacaacttgttctgcaaccacagaccttgctgttttgtggtcagtattggtatcatagcattttcatcctgacctggagccttcagtcaaaggcctcattgtgcagtaaggacgctggactcctgactcctatacttaaaacagacttggtaatttcaaacaagtcaaccagatgccagtatttctgcatgcatgtcttgtgggatggtgttgtgaggtcccctgacagatgcactgagtggccagggagacttttgtacccttttccattttaacagcccacgggtcactgtgttgcttccatcatattaacatcaacttgaaccagtggttcctgaaacacttcagttcattggaccttgctaattagcatcctgtaaaaacccaccaacaaatatcaactagacaggtagaatccaagtgaactgtacactcctggatcatgccagtaactgttttaataatacaccataaaatataactacgacttcattttacaaatctgtgtttaataaacaggtacaggcttgttgggtgcgaacttttaaaactcctaataaaaatgccagctatgattatctttgtttatg

SEQ ID NO: 12 - RNA, 3435 nt, rattus norvegicus
  source 1..3435: mol_type = mRNA
cgtttgtagtgtcagccatcccaattgcctgttccttctctgtgggagtggtgtctagacagtccaggcagggtatgctaggcaggtgcgttttggttgcctcagatcgcaacttgactccataacggtgaccaaagacaaaagaaggaaaccagattaaaaagaaccggacacagacccctgcagaatctggagcggccgtggttgggggcggggctacgacggggcggactcgggggcgtgggagggcggggccggggcggggcccggagccggctgcggttgcggtccctgcgccggcggtgaaggcgcagcggcggcgagtggctattgcaagcgtttggataatgtgagacctgggatgcagggatgtcgactatctgccccccaccatctcctgctgttgccaagacagagattgctttaagtggtgaatcacccttgttggcggctacctttgcttactgggataatattcttggtcctagagtaaggcacatttgggctccaaagacagaccaagtactcctcagtgatggagaaatcacttttcttgccaaccacactctgaatggagaaattcttcggaatgcggagagtggggcaatagatgtaaagttttttgtcttatctgaaaagggcgtcattattgtttcattaatcttcgacgggaactggaacggagatcggagcacttacggactatcaattatactgccgcagacggagctgagtttctacctcccactgcacagagtgtgtgttgacaggctaacgcacatcattcgaaaaggaaggatatggatgcacaaggaaagacaagaaaatgtccagaaaattgtcttggaaggcaccgagaggatggaagatcagggtcagagtatcatccctatgcttactggggaggtcatccctgtgatggagctgcttgcgtctatgagatcacacagtgttcctgaagacctcgatatagctgatacagtactcaatgatgatgacattggtgacagctgtcatgaaggctttcttctcaatgccatcagctcacatctgcagacctgcggctgttctgtggtggtaggcagcagtgcagagaaagtaaataagatagtaagaacactgtgcctttttctgacaccagcagagaggaagtgctccaggctgtgtgaagccgaatcgtcctttaaatacgaatctggactctttgtacaaggcttgctaaaggatgcgactggcagttttgtactacctttccggcaagttatgtatgccccttatcccaccacacacatcgatgtggatgtcaacactgtcaagcagatgccaccgtgtcatgaacatatttataatcaacgcagatacatgaggtcagagctgacagccttctggagggcaacttcagaagaggacatggctcaggacaccatcatctacacagatgagagcttcactcctgatttgaatattttccaagatgtcttacacagagacactctagtgaaagcctttctggatcaggtcttccatttgaagcctggcctgtctctcaggagtactttccttgcacagttcctcctcattcttcacagaaaagccttgacactaatcaagtacatagaggatgacacgcagaaggggaaaaagccctttaagtctcttcggaacctgaagatagatcttgatttaacagcagagggcgaccttaacataataatggctctagctgagaaaattaagccaggcctacactctttcatcttcgggagacctttctacactagtgtccaagaacgtgatgttctaatgactttttaaacatgtggtttgctccgtgtgtctcatgacagtcacacttgctgttacagtgtctcagcgctttggacacatccttcctccagggtcctgccgcaggacacgttacactacacttgtcagtagaggtctgtaccagatgtcaggtacatcgttgtagtgaatgtctcttttcctagactagatgtaccctcgtagggacttatgtttacaaccctcctaagtactagtgctgtcttgtaaggatacgaatgaagggatgtaaacttcaccacaactgctggttggttttgttgtttttgttttttgaaacttataattcatggtttacatgcatcacactgaaaccctagttagctttttacaggtaagctgtgagttgactgcctgtccctgtgttctctggcctgtacgatctgtggcgtgtaggatcacttttgcaacaactaaaaactaaagcactttgtttgcagttctacagaaagcaacttagtctgtctgcagattcgtttttgaaagaagacatgagaaagcggagttttaggtgaagtcagttgttggatcttcctttatagacttagtcctttagatgtggtctgtatagacatgcccaaccatcatgcatgggcactgaatatcgtgaactgtggtatgctttttgttggtttattgtacttctgtcaaagaaagtggcattggtttttataattgttgccaagttttaaggttaattttcattatttttgagccaaattaaaatgtgcacctcctgtgcctttcccaatcttggaaaatataatttcttggcagaaggtcagatttcagggcccagtcactttcgtctgacttccctttgcacagtccgccatgggcctggcttagaagttcttgtaaactatgccagagagtacattcgctgataaaatcttctttgcagagcaggagagcttcttgcctctttcctttcatttctgcctggactttggtgttctccacgttccctgcatcctaaggacagcaggagaactctgaccccagtgctatttctctaggtgctattgtggcaaactcaagcggtccgtctctgtccctgtaacgttcgtaccttgctggctgtgaagtactgactggtaaagctccgtgctacagcagtgtagggtatacacaaacacaagtaagtgttttatttaaaactgtggacttagcataaaaagggagactatatttattttttacaaaagggataaaaatggaaccctttcctcacccaccagatttagtcagaaaaaaacattctattctgaaaggtcacagtggttttgacatgacacatcagaacaacgcacactgtccatgatggcttatgaactccaagtcactccatcatggtaaatgggtagatccctccttctagtgtgccacaccattgcttcccacagtagaatcttatttaagtgctaagtgttgtctctgctggtttactctgttgttttagagaatgtaagttgtatagtgaataagttattgaagcatgtgtaaacactgttatacatcttttctcctagatggggaatttggaataaaatacctttaaaattcaaaaaaaaaaaaaaaaaaaaaaaa

SEQ ID NO: 13 - RNA, 36 nt, Homo sapiens
  source 1..36: mol_type = other RNA; note = 6 nucleotide repeat from intron1 of c9orf72
ggggccggggccggggccggggccggggccggggcc

SEQ ID NO: 14 - skipped

SEQ ID NO: 15 - DNA, 22 bp, synthetic construct
  source 1..22: mol_type = other DNA
tgtgacagttggaatgcagtga

SEQ ID NO: 16 - DNA, 25 bp, synthetic construct
  source 1..25: mol_type = other DNA
gccacttaaagcaatctctgtcttg

SEQ ID NO: 17 - DNA, 21 bp, synthetic construct
  source 1..21: mol_type = other DNA
tcgactctttgcccaccgcca

SEQ ID NO: 18 - DNA, 20 bp, synthetic construct
  source 1..20: mol_type = other DNA
gggtctagcaagagcaggtg

SEQ ID NO: 19 - DNA, 21 bp, synthetic construct
  source 1..21: mol_type = other DNA
gtcttggcaacagctggagat

SEQ ID NO: 20 - DNA, 24 bp, synthetic construct
  source 1..24: mol_type = other DNA
tgatgtcgactctttgcccaccgc

SEQ ID NO: 21 - DNA, 22 bp, synthetic construct
  source 1..22: mol_type = other DNA
tcctgtaatggaactgctttca

SEQ ID NO: 22 - DNA, 22 bp, synthetic construct
  source 1..22: mol_type = other DNA
ggtatctgcttcatccagcttt

SEQ ID NO: 23 - DNA, 16 bp, synthetic construct
  source 1..16: mol_type = other DNA
ccccggccccggcccc

SEQ ID NO: 24 - DNA, 20 bp, synthetic construct
  source 1..20: mol_type = other DNA
caagccaccgtctcactcaa

SEQ ID NO: 25 - DNA, 28 bp, synthetic construct
  source 1..28: mol_type = other DNA
gtagtgctgtctactccagagagttacc

SEQ ID NO: 26 - DNA, 30 bp, synthetic construct
  source 1..30: mol_type = other DNA
cttggcttccctcaaaagactggctaatgt

SEQ ID NO: 27-29 - skipped (3 entries)

SEQ ID NO: 30 - DNA, 20 bp, synthetic construct
  source 1..20: mol_type = other DNA
agcgggacaccgtaggttac

SEQ ID NO: 31 - DNA, 20 bp, synthetic construct
  source 1..20: mol_type = other DNA
gtgggcggaacttgtcgctg

SEQ ID NO: 32 - DNA, 20 bp, synthetic construct
  source 1..20: mol_type = other DNA
gtcacattatccaaatgctc

SEQ ID NO: 33 - DNA, 20 bp, synthetic construct
  source 1..20: mol_type = other DNA
ggtgggcaaagagtcgacat

SEQ ID NO: 34 - DNA, 20 bp, synthetic construct
  source 1..20: mol_type = other DNA
atctctgtcttggcaacagc

SEQ ID NO: 35 - DNA, 20 bp, synthetic construct
  source 1..20: mol_type = other DNA
aagcaatctctgtcttggca

SEQ ID NO: 36 - DNA, 20 bp, synthetic construct
  source 1..20: mol_type = other DNA
acttaaagcaatctctgtct

SEQ ID NO: 37 - DNA, 20 bp, synthetic construct
  source 1..20: mol_type = other DNA
ttgccacttaaagcaatctc

SEQ ID NO: 38 - DNA, 20 bp, synthetic construct
  source 1..20: mol_type = other DNA
cccagtaagcaaaagtagct

SEQ ID NO: 39 - DNA, 20 bp, synthetic construct
  source 1..20: mol_type = other DNA
actctaggaccaagaatatt

SEQ ID NO: 40 - DNA, 20 bp, synthetic construct
  source 1..20: mol_type = other DNA
gccttactctaggaccaaga

SEQ ID NO: 41 - DNA, 20 bp, synthetic construct
  source 1..20: mol_type = other DNA
ccaaatgtgccttactctag

SEQ ID NO: 42 - DNA, 20 bp, synthetic construct
  source 1..20: mol_type = other DNA
tggagcccaaatgtgcctta

SEQ ID NO: 43 - DNA, 20 bp, synthetic construct
  source 1..20: mol_type = other DNA
tctgtctttggagcccaaat

SEQ ID NO: 44 - DNA, 20 bp, synthetic construct
  source 1..20: mol_type = other DNA
ccatcactgagaagtacctg

SEQ ID NO: 45 - DNA, 20 bp, synthetic construct
  source 1..20: mol_type = other DNA
atttctccatcactgagaag

SEQ ID NO: 46 - DNA, 20 bp, synthetic construct
  source 1..20: mol_type = other DNA
aaaagttatttctccatcac

SEQ ID NO: 47 - DNA, 20 bp, synthetic construct
  source 1..20: mol_type = other DNA
tggcaagaaaagttatttct

SEQ ID NO: 48 - DNA, 20 bp, synthetic construct
  source 1..20: mol_type = other DNA
gtgtggttggcaagaaaagt

SEQ ID NO: 49 - DNA, 20 bp, synthetic construct
  source 1..20: mol_type = other DNA
ctccatttagagtgtggttg

SEQ ID NO: 50 - DNA, 20 bp, synthetic construct
  source 1..20: mol_type = other DNA
tgcatttcgaaggatttctc

SEQ ID NO: 51 - DNA, 20 bp, synthetic construct
  source 1..20: mol_type = other DNA
ccactctctgcatttcgaag

SEQ ID NO: 52 - DNA, 20 bp, synthetic construct
  source 1..20: mol_type = other DNA
acaaaaaactttacatctat

SEQ ID NO: 53 - DNA, 20 bp, synthetic construct
  source 1..20: mol_type = other DNA
ccttttcagacaagacaaaa

SEQ ID NO: 54 - DNA, 20 bp, synthetic construct
  source 1..20: mol_type = other DNA
aagattaatgaaacaataat

SEQ ID NO: 55 - DNA, 20 bp, synthetic construct
  source 1..20: mol_type = other DNA
gtttccatcaaagattaatg

SEQ ID NO: 56 - DNA, 20 bp, synthetic construct
  source 1..20: mol_type = other DNA
attgatagtccatatgtgct

SEQ ID NO: 57 - DNA, 20 bp, synthetic construct
  source 1..20: mol_type = other DNA
agtataattgatagtccata

SEQ ID NO: 58 - DNA, 20 bp, synthetic construct
  source 1..20: mol_type = other DNA
ggaggtagaaactaagttct

SEQ ID NO: 59 - DNA, 20 bp, synthetic construct
  source 1..20: mol_type = other DNA
atgtgttaatctatcaacac

SEQ ID NO: 60 - DNA, 20 bp, synthetic construct
  source 1..20: mol_type = other DNA
tgcatccatattcttccttt

SEQ ID NO: 61 - DNA, 20 bp, synthetic construct
  source 1..20: mol_type = other DNA
ttccttatgcatccatattc

SEQ ID NO: 62 - DNA, 20 bp, synthetic construct
  source 1..20: mol_type = other DNA
cttgtctttccttatgcatc

SEQ ID NO: 63 - DNA, 20 bp, synthetic construct
  source 1..20: mol_type = other DNA
acattttcttgtctttcctt

SEQ ID NO: 64 - DNA, 20 bp, synthetic construct
  source 1..20: mol_type = other DNA
tctggacattttcttgtctt

SEQ ID NO: 65 - DNA, 20 bp, synthetic construct
  source 1..20: mol_type = other DNA
ataatcttctggacattttc

SEQ ID NO: 66 - DNA, 20 bp, synthetic construct
  source 1..20: mol_type = other DNA
ctctgaccctgatcttccat

SEQ ID NO: 67 - DNA, 20 bp, synthetic construct
  source 1..20: mol_type = other DNA
ttggaataatactctgaccc

SEQ ID NO: 68 - DNA, 20 bp, synthetic construct
  source 1..20: mol_type = other DNA
cagttccattacaggaatca

SEQ ID NO: 69 - DNA, 20 bp, synthetic construct
  source 1..20: mol_type = other DNA
cttcaggaacactgtgtgat

SEQ ID NO: 70 - DNA, 20 bp, synthetic construct
  source 1..20: mol_type = other DNA
atctatttcttcaggaacac

SEQ ID NO: 71 - DNA, 20 bp, synthetic construct
  source 1..20: mol_type = other DNA
agtactgtatcagctatatc

SEQ ID NO: 72 - DNA, 20 bp, synthetic construct
  source 1..20: mol_type = other DNA
tcattgagtactgtatcagc

SEQ ID NO: 73 - DNA, 20 bp, synthetic construct
  source 1..20: mol_type = other DNA
tcatcatcattgagtactgt

SEQ ID NO: 74 - DNA, 20 bp, synthetic construct
  source 1..20: mol_type = other DNA
ccaatatcatcatcattgag

SEQ ID NO: 75 - DNA, 20 bp, synthetic construct
  source 1..20: mol_type = other DNA
tcatgacagctgtcaccaat

SEQ ID NO: 76 - DNA, 20 bp, synthetic construct
  source 1..20: mol_type = other DNA
aagccttcatgacagctgtc

SEQ ID NO: 77 - DNA, 20 bp, synthetic construct
  source 1..20: mol_type = other DNA
agaagaaagccttcatgaca

SEQ ID NO: 78 - DNA, 20 bp, synthetic construct
  source 1..20: mol_type = other DNA
tacttgagaagaaagccttc

SEQ ID NO: 79 - DNA, 20 bp, synthetic construct
  source 1..20: mol_type = other DNA
attcttacttgagaagaaag

SEQ ID NO: 80 - DNA, 20 bp, synthetic construct
  source 1..20: mol_type = other DNA
aaaaattcttacttgagaag

SEQ ID NO: 81 - DNA, 20 bp, synthetic construct
  source 1..20: mol_type = other DNA
agatggtatctgcttcatcc

SEQ ID NO: 82 - DNA, 20 bp, synthetic construct
  source 1..20: mol_type = other DNA
caatctaagtagacagtctg

SEQ ID NO: 83 - DNA, 20 bp, synthetic construct
  source 1..20: mol_type = other DNA
ttaagcaacagttcaaatac

SEQ ID NO: 84 - DNA, 20 bp, synthetic construct
  source 1..20: mol_type = other DNA
ctttaaatagcaaatggaat

SEQ ID NO: 85 - DNA, 20 bp, synthetic construct
  source 1..20: mol_type = other DNA
gccatgatttcttgtctggg

SEQ ID NO: 86 - DNA, 20 bp, synthetic construct
  source 1..20: mol_type = other DNA
gctttaatgagaagtaaaac

SEQ ID NO: 87 - DNA, 20 bp, synthetic construct
  source 1..20: mol_type = other DNA
tctacagtacaacttaatat

SEQ ID NO: 88 - DNA, 20 bp, synthetic construct
  source 1..20: mol_type = other DNA
ataattttgttctacgccta

SEQ ID NO: 89 - DNA, 20 bp, synthetic construct
  source 1..20: mol_type = other DNA
cactgctggatggaaaaaga

SEQ ID NO: 90 - DNA, 20 bp, synthetic construct
  source 1..20: mol_type = other DNA
tggtttaagggcacaaactc

SEQ ID NO: 91 - DNA, 20 bp, synthetic construct
  source 1..20: mol_type = other DNA
ttgcccacgggtacacagca

SEQ ID NO: 92 - DNA, 20 bp, synthetic construct
  source 1..20: mol_type = other DNA
cagatgaggaaataggtgta

SEQ ID NO: 93 - DNA, 20 bp, synthetic construct
  source 1..20: mol_type = other DNA
acacattaggtactattact

SEQ ID NO: 94 - DNA, 20 bp, synthetic construct
  source 1..20: mol_type = other DNA
tttttatgttccaggcactg

SEQ ID NO: 95 - DNA, 20 bp, synthetic construct
  source 1..20: mol_type = other DNA
aataggaaatgttagctatg

SEQ ID NO: 96 - DNA, 20 bp, synthetic construct
  source 1..20: mol_type = other DNA
ggcactcaacaaatactggc

SEQ ID NO: 97 - DNA, 20 bp, synthetic construct
  source 1..20: mol_type = other DNA
tacatgtaaagcaactagta

SEQ ID NO: 98 - DNA, 20 bp, synthetic construct
  source 1..20: mol_type = other DNA
taaaatttcatgaaaatctg

SEQ ID NO: 99 - DNA, 20 bp, synthetic construct
  source 1..20: mol_type = other DNA
aagtgaatactttatacttt

SEQ ID NO: 100 - DNA, 20 bp, synthetic construct
  source 1..20: mol_type = other DNA
catcatgagcctaaaggaaa

SEQ ID NO: 101 - DNA, 20 bp, synthetic construct
  source 1..20: mol_type = other DNA
ggctcttaggttaaacacac

SEQ ID NO: 102 - DNA, 20 bp, synthetic construct
  source 1..20: mol_type = other DNA
tgcttctgattcaagccatt

SEQ ID NO: 103 - DNA, 20 bp, synthetic construct
  source 1..20: mol_type = other DNA
atacaggactaaagtgcttc

SEQ ID NO: 104 - DNA, 20 bp, synthetic construct
  source 1..20: mol_type = other DNA
caaatgggatttaaaatgat

SEQ ID NO: 105 - DNA, 20 bp, synthetic construct
  source 1..20: mol_type = other DNA
tgacatgtagagagattaag

SEQ ID NO: 106 - DNA, 20 bp, synthetic construct
  source 1..20: mol_type = other DNA
ttattgaaataccatcattt

SEQ ID NO: 107 - DNA, 20 bp, synthetic construct
  source 1..20: mol_type = other DNA
tagtcagtataatatcattt

SEQ ID NO: 108 - DNA, 20 bp, synthetic construct
  source 1..20: mol_type = other DNA
gcattgagaagaaagccttc

SEQ ID NO: 109 - DNA, 20 bp, synthetic construct
  source 1..20: mol_type = other DNA
aagacctgatccaggaaggc

SEQ ID NO: 110 - DNA, 20 bp, synthetic construct
  source 1..20: mol_type = other DNA
tgagctgatggcattgagaa

SEQ ID NO: 111 - DNA, 20 bp, synthetic construct
  source 1..20: mol_type = other DNA
acaacggaacagccacaggt

SEQ ID NO: 112 - DNA, 20 bp, synthetic construct
  source 1..20: mol_type = other DNA
ttagtgtcaaggcttttctg

SEQ ID NO: 113 - DNA, 20 bp, synthetic construct
  source 1..20: mol_type = other DNA
gacggctgacacaccaagcg

SEQ ID NO: 114 - DNA, 20 bp, synthetic construct
  source 1..20: mol_type = other DNA
tgatggcattgagaagaaag

SEQ ID NO: 115 - DNA, 20 bp, synthetic construct
  source 1..20: mol_type = other DNA
tttactttctctgcactgct

SEQ ID NO: 116 - DNA, 20 bp, synthetic construct
  source 1..20: mol_type = other DNA
tcttatttactttctctgca

SEQ ID NO: 117 - DNA, 20 bp, synthetic construct
  source 1..20: mol_type = other DNA
ggcataatgttctgactatc

SEQ ID NO: 118 - DNA, 20 bp, synthetic construct
  source 1..20: mol_type = other DNA
ataacctggagcattttctc

SEQ ID NO: 119 - DNA, 20 bp, synthetic construct
  source 1..20: mol_type = other DNA
ccctgactcatatttaaatg

SEQ ID NO: 120 - DNA, 20 bp, synthetic construct
  source 1..20: mol_type = other DNA
ccagttgaatcctttagcag

SEQ ID NO: 121 - DNA, 20 bp, synthetic construct
  source 1..20: mol_type = other DNA
catacatgacttgccggaaa

SEQ ID NO: 122 - DNA, 20 bp, synthetic construct
  source 1..20: mol_type = other DNA
gacatccacatctatgtgtg

SEQ ID NO: 123 - DNA, 20 bp, synthetic construct
  source 1..20: mol_type = other DNA
tgttcatgacagggtggcat

SEQ ID NO: 124 - DNA, 20 bp, synthetic construct
  source 1..20: mol_type = other DNA
ttataaatatgttcatgaca

SEQ ID NO: 125 - DNA, 20 bp, synthetic construct
  source 1..20: mol_type = other DNA
cagctcggatctcatgtatc

SEQ ID NO: 126 - DNA, 20 bp, synthetic construct
  source 1..20: mol_type = other DNA
ctccagaaggctgtcagctc

SEQ ID NO: 127 - DNA, 20 bp, synthetic construct
  source 1..20: mol_type = other DNA
gtatcctgagccatgtcttc

SEQ ID NO: 128 - DNA, 20 bp, synthetic construct
  source 1..20: mol_type = other DNA
aatcaggagtaaagctttcg

SEQ ID NO: 129 - DNA, 20 bp, synthetic construct
  source 1..20: mol_type = other DNA
aaaatattcaaatcaggagt

SEQ ID NO: 130 - DNA, 20 bp, synthetic construct
  source 1..20: mol_type = other DNA
tctctgtgtaagacatcttg

SEQ ID NO: 131 - DNA, 20 bp, synthetic construct
  source 1..20: mol_type = other DNA
gagtgtctctgtgtaagaca

SEQ ID NO: 132 - DNA, 20 bp, synthetic construct
  source 1..20: mol_type = other DNA
cactagagtgtctctgtgta

SEQ ID NO: 133 - DNA, 20 bp, synthetic construct
  source 1..20: mol_type = other DNA
gctttcactagagtgtctct

SEQ ID NO: 134 - DNA, 20 bp, synthetic construct
  source 1..20: mol_type = other DNA
gatccaggaaggctttcact

SEQ ID NO: 135 - DNA, 20 bp, synthetic construct
  source 1..20: mol_type = other DNA
aaagtacttctgagagataa

SEQ ID NO: 136 - DNA, 20 bp, synthetic construct
  source 1..20: mol_type = other DNA
aactgtgcaaggaaagtact

SEQ ID NO: 137 - DNA, 20 bp, synthetic construct
  source 1..20: mol_type = other DNA
gtcaaggcttttctgtgaag

SEQ ID NO: 138 - DNA, 20 bp, synthetic construct
  source 1..20: mol_type = other DNA
agagatttaaagggcttttt

SEQ ID NO: 139 - DNA, 20 bp, synthetic construct
  source 1..20: mol_type = other DNA
atcttcaggttccgaagaga

SEQ ID NO: 140 - DNA, 20 bp, synthetic construct
  source 1..20: mol_type = other DNA
ccctctgctgttaaatcaag

SEQ ID NO: 141 - DNA, 20 bp, synthetic construct
  source 1..20: mol_type = other DNA
tgttaagatcgccctctgct

SEQ ID NO: 142 - DNA, 20 bp, synthetic construct
  source 1..20: mol_type = other DNA
attattatgttaagatcgcc

SEQ ID NO: 143 - DNA, 20 bp, synthetic construct
  source 1..20: mol_type = other DNA
agagccattattatgttaag

SEQ ID NO: 144 - DNA, 20 bp, synthetic construct
  source 1..20: mol_type = other DNA
ataaaagagtgtaggcctgg

SEQ ID NO: 145 - DNA, 20 bp, synthetic construct
  source 1..20: mol_type = other DNA
acactagtgtagaaaggtct

SEQ ID NO: 146 - DNA, 20 bp, synthetic construct
  source 1..20: mol_type = other DNA
gttcttgcacactagtgtag

SEQ ID NO: 147 - DNA, 20 bp, synthetic construct
  source 1..20: mol_type = other DNA
taaaaagtcattagaacatc

SEQ ID NO: 148 - DNA, 20 bp, synthetic construct
  source 1..20: mol_type = other DNA
tattaagttacacatttaaa

SEQ ID NO: 149 - DNA, 20 bp, synthetic construct
  source 1..20: mol_type = other DNA
ctttaccagcgatcatgatt

SEQ ID NO: 150 - DNA, 20 bp, synthetic construct
  source 1..20: mol_type = other DNA
ttctggagtatgatccaggg

SEQ ID NO: 151 - DNA, 20 bp, synthetic construct
  source 1..20: mol_type = other DNA
acttaactgcaattgctgag

SEQ ID NO: 152 - DNA, 20 bp, synthetic construct
  source 1..20: mol_type = other DNA
tgtagtgtaacttacttaac

SEQ ID NO: 153 - DNA, 20 bp, synthetic construct
  source 1..20: mol_type = other DNA
atgcacctgacatcccctca

SEQ ID NO: 154 - DNA, 20 bp, synthetic construct
  source 1..20: mol_type = other DNA
cccaaaagcataaatctagg

SEQ ID NO: 155 - DNA, 20 bp, synthetic construct
  source 1..20: mol_type = other DNA
atatttattatattgtaaac

SEQ ID NO: 156 - DNA, 20 bp, synthetic construct
  source 1..20: mol_type = other DNA
agcaataatatttattatat

SEQ ID NO: 157 - DNA, 20 bp, synthetic construct
  source 1..20: mol_type = other DNA
agatagcaataatatttatt

SEQ ID NO: 158 - DNA, 20 bp, synthetic construct
  source 1..20: mol_type = other DNA
aaagatagcaataatattta

SEQ ID NO: 159 - DNA, 20 bp, synthetic construct
  source 1..20: mol_type = other DNA
ttaaaagatagcaataatat

SEQ ID NO: 160 - DNA, 20 bp, synthetic construct
  source 1..20: mol_type = other DNA
atctttaaaagatagcaata

SEQ ID NO: 161 - DNA, 20 bp, synthetic construct
  source 1..20: mol_type = other DNA
atatctttaaaagatagcaa

SEQ ID NO: 162 - DNA, 20 bp, synthetic construct
  source 1..20: mol_type = other DNA
attatatctttaaaagatag

SEQ ID NO: 163 - DNA, 20 bp, synthetic construct
  source 1..20: mol_type = other DNA
tattattatatctttaaaag

SEQ ID NO: 164 - DNA, 20 bp, synthetic construct
  source 1..20: mol_type = other DNA
caagtttacatcctattatt

SEQ ID NO: 165 - DNA, 20 bp, synthetic construct
  source 1..20: mol_type = other DNA
aaaacagtagttgtggtcaa

SEQ ID NO: 166 - DNA, 20 bp, synthetic construct
  source 1..20: mol_type = other DNA
aaaaaacagtagttgtggtc

SEQ ID NO: 167 - DNA, 20 bp, synthetic construct
  source 1..20: mol_type = other DNA
tgaatcatgtatttcaaaaa

SEQ ID NO: 168 - DNA, 20 bp, synthetic construct
  source 1..20: mol_type = other DNA
gccaactcagatttcacctt

SEQ ID NO: 169 - DNA, 20 bp, synthetic construct
  source 1..20: mol_type = other DNA
ctacacaccaaagaatgcca

SEQ ID NO: 170 - DNA, 20 bp, synthetic construct
  source 1..20: mol_type = other DNA
agttttcagttgattgcaga

SEQ ID NO: 171 - DNA, 20 bp, synthetic construct
  source 1..20: mol_type = other DNA
catcctatgttcaagctcac

SEQ ID NO: 172 - DNA, 20 bp, synthetic construct
  source 1..20: mol_type = other DNA
taaacatctgcttgatcaat

SEQ ID NO: 173 - DNA, 20 bp, synthetic construct
  source 1..20: mol_type = other DNA
aatccacaaagtaggatcta

SEQ ID NO: 174 - DNA, 20 bp, synthetic construct
  source 1..20: mol_type = other DNA
attagacatttctacagact

SEQ ID NO: 175 - DNA, 20 bp, synthetic construct
  source 1..20: mol_type = other DNA
ctcaactacatagaatatca

SEQ ID NO: 176 - DNA, 20 bp, synthetic construct
  source 1..20: mol_type = other DNA
ttggcaacaattactaaaac

SEQ ID NO: 177 - DNA, 20 bp, synthetic construct
  source 1..20: mol_type = other DNA
tcaaaaataatgaaaattaa

SEQ ID NO: 178 - DNA, 20 bp, synthetic construct
  source 1..20: mol_type = other DNA
caatttggctcaaaaataat

SEQ ID NO: 179 - DNA, 20 bp, synthetic construct
  source 1..20: mol_type = other DNA
ggcacaggaggtgcacattt

SEQ ID NO: 180 - DNA, 20 bp, synthetic construct
  source 1..20: mol_type = other DNA
tagattttctaaggagaaaa

SEQ ID NO: 181 - DNA, 20 bp, synthetic construct
  source 1..20: mol_type = other DNA
actgaccagtgaaatctgaa

SEQ ID NO: 182 - DNA, 20 bp, synthetic construct
  source 1..20: mol_type = other DNA
ggtaagacttagcaagaaga

SEQ ID NO: 183 - DNA, 20 bp, synthetic construct
  source 1..20: mol_type = other DNA
tctcagagttgcaatgattg

SEQ ID NO: 184 - DNA, 20 bp, synthetic construct
  source 1..20: mol_type = other DNA
agatcttattagttagtata

SEQ ID NO: 185 - DNA, 20 bp, synthetic construct
  source 1..20: mol_type = other DNA
agtactcaaggaactatttt

SEQ ID NO: 186 - DNA, 20 bp, synthetic construct
  source 1..20: mol_type = other DNA
ggcaaacagcaacaacttca

SEQ ID NO: 187 - DNA, 20 bp, synthetic construct
  source 1..20: mol_type = other DNA
gcacttcagtaaaatttctc

SEQ ID NO: 188 - DNA, 20 bp, synthetic construct
  source 1..20: mol_type = other DNA
ggtccaaacgcattaagaaa

SEQ ID NO: 189 - DNA, 20 bp, synthetic construct
  source 1..20: mol_type = other DNA
gaattatattaatcagttat

SEQ ID NO: 190 - DNA, 20 bp, synthetic construct
  source 1..20: mol_type = other DNA
tgtgtttgtgtaactacaat

SEQ ID NO: 191 - DNA, 20 bp, synthetic construct
  source 1..20: mol_type = other DNA
atattacttccagaatttta

SEQ ID NO: 192 - DNA, 20 bp, synthetic construct
  source 1..20: mol_type = other DNA
ggcagaagggctctattacc

SEQ ID NO: 193 - DNA, 20 bp, synthetic construct
  source 1..20: mol_type = other DNA
cattcgaacatgtcattttg

SEQ ID NO: 194 - DNA, 20 bp, synthetic construct
  source 1..20: mol_type = other DNA
ctgattcatgatgggaaagc

SEQ ID NO: 195 - DNA, 20 bp, synthetic construct
  source 1..20: mol_type = other DNA
gtggttgtctaaaacatcaa

SEQ ID NO: 196 - DNA, 20 bp, synthetic construct
  source 1..20: mol_type = other DNA
atgactgagctacagtacaa

SEQ ID NO: 197 - DNA, 20 bp, synthetic construct
  source 1..20: mol_type = other DNA
gggacactacaaggtagtat

SEQ ID NO: 198 - DNA, 20 bp, synthetic construct
  source 1..20: mol_type = other DNA
ttaaataagaatctaccatg

SEQ ID NO: 199 - DNA, 20 bp, synthetic construct
  source 1..20: mol_type = other DNA
gctttaataacttatttcac

SEQ ID NO: 200 - DNA, 20 bp, synthetic construct
  source 1..20: mol_type = other DNA
aggagaaaagatatataaca

SEQ ID NO: 201 - DNA, 20 bp, synthetic construct
  source 1..20: mol_type = other DNA
ccatttaggagaaaagatat

SEQ ID NO: 202 - DNA, 20 bp, synthetic construct
  source 1..20: mol_type = other DNA
ttcaccctcagcgagtactg

SEQ ID NO: 203 - DNA, 20 bp, synthetic construct
  source 1..20: mol_type = other DNA
aggctgcggttgtttccctc

SEQ ID NO: 204 - DNA, 20 bp, synthetic construct
  source 1..20: mol_type = other DNA
gccagatccccatcccttgt

SEQ ID NO: 205 - DNA, 20 bp, synthetic construct
  source 1..20: mol_type = other DNA
tcacttcctttaagcaagtc

SEQ ID NO: 206 - DNA, 20 bp, synthetic construct
  source 1..20: mol_type = other DNA
agtgatgcccaagtcacaat

SEQ ID NO: 207 - DNA, 20 bp, synthetic construct
  source 1..20: mol_type = other DNA
agtcaagtgatgcccaagtc

SEQ ID NO: 208 - DNA, 20 bp, synthetic construct
  source 1..20: mol_type = other DNA
ccatcagtcaagtgatgccc

SEQ ID NO: 209 - DNA, 20 bp, synthetic construct
  source 1..20: mol_type = other DNA
gattaccatcagtcaagtga

SEQ ID NO: 210 - DNA, 20 bp, synthetic construct
  source 1..20: mol_type = other DNA
caactgattaccatcagtca

SEQ ID NO: 211 - DNA, 20 bp, synthetic construct
  source 1..20: mol_type = other DNA
gcagtttccaactgattcag

SEQ ID NO: 212 - DNA, 20 bp, synthetic construct
  source 1..20: mol_type = other DNA
cgttcttgtttcagatgtac

SEQ ID NO: 213 - DNA, 20 bp, synthetic construct
  source 1..20: mol_type = other DNA
gccaaacaaaatattttatc

SEQ ID NO: 214 - DNA, 20 bp, synthetic construct
  source 1..20: mol_type = other DNA
taggtaggctaacctagtcc

SEQ ID NO: 215 - DNA, 20 bp, synthetic construct
  source 1..20: mol_type = other DNA
tcccagcccaaagagaagca

SEQ ID NO: 216 - DNA, 20 bp, synthetic construct
  source 1..20: mol_type = other DNA
ggatcatagctctcggtaac

SEQ ID NO: 217 - DNA, 20 bp, synthetic construct
  source 1..20: mol_type = other DNA
aatcataaagccctcacttc

SEQ ID NO: 218 - DNA, 20 bp, synthetic construct
  source 1..20: mol_type = other DNA
ctgattggtatttagaaagg

SEQ ID NO: 219 - DNA, 20 bp, synthetic construct
  source 1..20: mol_type = other DNA
atgcagacatgattacatta

SEQ ID NO: 220 - DNA, 20 bp, synthetic construct
  source 1..20: mol_type = other DNA
ttcatcattaaactgaaaat

SEQ ID NO: 221 - DNA, 20 bp, synthetic construct
  source 1..20: mol_type = other DNA
cttttaggttaaaaaggtgg

SEQ ID NO: 222 - DNA, 20 bp, synthetic construct
  source 1..20: mol_type = other DNA
atacagagcctggcaaaaca

SEQ ID NO: 223 - DNA, 20 bp, synthetic construct
  source 1..20: mol_type = other DNA
ttctatttacagagcattag

SEQ ID NO: 224 - DNA, 20 bp, synthetic construct
  source 1..20: mol_type = other DNA
gccttcacattaattcacca

SEQ ID NO: 225 - DNA, 20 bp, synthetic construct
  source 1..20: mol_type = other DNA
tgtgttattgcccctaaaaa

SEQ ID NO: 226 - DNA, 20 bp, synthetic construct
  source 1..20: mol_type = other DNA
tgtattcactatactatgcc

SEQ ID NO: 227 - DNA, 20 bp, synthetic construct
  source 1..20: mol_type = other DNA
aagttatttaaagtatagca

SEQ ID NO: 228 - DNA, 20 bp, synthetic construct
  source 1..20: mol_type = other DNA
gacattgaagtatcaagaca

SEQ ID NO: 229 - DNA, 20 bp, synthetic construct
  source 1..20: mol_type = other DNA
tgttaagtaatcttagaaaa

SEQ ID NO: 230 - DNA, 20 bp, synthetic construct
  source 1..20: mol_type = other DNA
ggcatacatttagaaattca

SEQ ID NO: 231 - DNA, 20 bp, synthetic construct
  source 1..20: mol_type = other DNA
accttatgcatccatattct

SEQ ID NO: 232 - DNA, 20 bp, synthetic construct
  source 1..20: mol_type = other DNA
gaattctcttgggaaccatt

SEQ ID NO: 233 - DNA, 20 bp, synthetic construct
  source 1..20: mol_type = other DNA
atattcaactacaggattta

SEQ ID NO: 234 - DNA, 20 bp, synthetic construct
  source 1..20: mol_type = other DNA
atgtgttctttagatacatc

SEQ ID NO: 235 - DNA, 20 bp, synthetic construct
  source 1..20: mol_type = other DNA
ccttatacagatacatgctg

SEQ ID NO: 236 - DNA, 20 bp, synthetic construct
  source 1..20: mol_type = other DNA
tagatgcaattactattttc

SEQ ID NO: 237 - DNA, 20 bp, synthetic construct
  source 1..20: mol_type = other DNA
tgtacttcccaaacttgaac

SEQ ID NO: 238 - DNA, 20 bp, synthetic construct
  source 1..20: mol_type = other DNA
ctgaagctcaacaacaccaa

SEQ ID NO: 239 - DNA, 20 bp, synthetic construct
  source 1..20: mol_type = other DNA
gtctatagaatcaaactgaa

SEQ ID NO: 240 - DNA, 20 bp, synthetic construct
  source 1..20: mol_type = other DNA
ttgaatcaatacctaacctc

SEQ ID NO: 241 - DNA, 20 bp, synthetic construct
  source 1..20: mol_type = other DNA
tgcctcttttagaaaagatc

SEQ ID NO: 242 - DNA, 20 bp, synthetic construct
  source 1..20: mol_type = other DNA
atggaatcattggtttatcg

SEQ ID NO: 243 - DNA, 20 bp, synthetic construct
  source 1..20: mol_type = other DNA
aaagctcacttttattcttt

SEQ ID NO: 244 - DNA, 20 bp, synthetic construct
  source 1..20: mol_type = other DNA
ggtgccgccaccatgcccgg

SEQ ID NO: 245 - DNA, 20 bp, synthetic construct
  source 1..20: mol_type = other DNA
gagagaagctgggcaataaa

SEQ ID NO: 246 - DNA, 20 bp, synthetic construct
  source 1..20: mol_type = other DNA
tctgaccctgcacaataaag

SEQ ID NO: 247 - DNA, 20 bp, synthetic construct
  source 1..20: mol_type = other DNA
atagtgtgtgattcaaaacg

SEQ ID NO: 248 - DNA, 20 bp, synthetic construct
  source 1..20: mol_type = other DNA
actgtatcagctatctaaaa

SEQ ID NO: 249 - DNA, 20 bp, synthetic construct
  source 1..20: mol_type = other DNA
ttatttgtataggaacctac

SEQ ID NO: 250 - DNA, 20 bp, synthetic construct
  source 1..20: mol_type = other DNA
tgtgagctgatggcactgta

SEQ ID NO: 251 - DNA, 20 bp, synthetic construct
  source 1..20: mol_type = other DNA
ccttatttactttctctgca

SEQ ID NO: 252 - DNA, 20 bp, synthetic construct
  source 1..20: mol_type = other DNA
ggaataaggtcactagttcg

SEQ ID NO: 253 - DNA, 20 bp, synthetic construct
  source 1..20: mol_type = other DNA
atttgcaacaatttttaaat

SEQ ID NO: 254 - DNA, 20 bp, synthetic construct
  source 1..20: mol_type = other DNA
ataaactaccaatgatatcc

SEQ ID NO: 255 - DNA, 20 bp, synthetic construct
  source 1..20: mol_type = other DNA
tacctgatccaggaaggctt

SEQ ID NO: 256 - DNA, 20 bp, synthetic construct
  source 1..20: mol_type = other DNA
ttcccgaagcataaatctag

SEQ ID NO: 257 - DNA, 20 bp, synthetic construct
  source 1..20: mol_type = other DNA
ttgagaagcatgaaattcca

SEQ ID NO: 258 - DNA, 20 bp, synthetic construct
  source 1..20: mol_type = other DNA
gcgggacaccgtaggttacg

SEQ ID NO: 259 - DNA, 20 bp, synthetic construct
  source 1..20: mol_type = other DNA
ctttcctagcgggacaccgt

SEQ ID NO: 260 - DNA, 20 bp, synthetic construct
  source 1..20: mol_type = other DNA
gcacctctctttcctagcgg

SEQ ID NO: 261 - DNA, 20 bp, synthetic construct
  source 1..20: mol_type = other DNA
tgtttgacgcacctctcttt

SEQ ID NO: 262 - DNA, 20 bp, synthetic construct
  source 1..20: mol_type = other DNA
cttgtcgctgtttgacgcac

SEQ ID NO: 263 - DNA, 20 bp, synthetic construct
  source 1..20: mol_type = other DNA
gggcggaacttgtcgctgtt

SEQ ID NO: 264 - DNA, 20 bp, synthetic construct
  source 1..20: mol_type = other DNA
gcagcagggacggctgacac

SEQ ID NO: 265 - DNA, 20 bp, synthetic construct
  source 1..20: mol_type = other DNA
agaagcaaccgggcagcagg

SEQ ID NO: 266 - DNA, 20 bp, synthetic construct
  source 1..20: mol_type = other DNA
cccaaaagagaagcaaccgg

SEQ ID NO: 267 - DNA, 20 bp, synthetic construct
  source 1..20: mol_type = other DNA
accccgcccccaaaagagaa

SEQ ID NO: 268 - DNA, 20 bp, synthetic construct
  source 1..20: mol_type = other DNA
cttgctagaccccgccccca

SEQ ID NO: 269 - DNA, 20 bp, synthetic construct
  source 1..20: mol_type = other DNA
cacctgctcttgctagaccc

SEQ ID NO: 270 - DNA, 20 bp, synthetic construct
  source 1..20: mol_type = other DNA
taaacccacacctgctcttg

SEQ ID NO: 271 - DNA, 20 bp, synthetic construct
  source 1..20: mol_type = other DNA
ctcctaaacccacacctgct

SEQ ID NO: 272 - DNA, 20 bp, synthetic construct
  source 1..20: mol_type = other DNA
acacacacctcctaaaccca

SEQ ID NO: 273 - DNA, 20 bp, synthetic construct
  source 1..20: mol_type = other DNA
aaacaaaaacacacacctcc

SEQ ID NO: 274 - DNA, 20 bp, synthetic construct
  source 1..20: mol_type = other DNA
ggtgggaaaaacaaaaacac

SEQ ID NO: 275 - DNA, 20 bp, synthetic construct
  source 1..20: mol_type = other DNA
ctgtgagagcaagtagtggg

SEQ ID NO: 276 - DNA, 20 bp, synthetic construct
  source 1..20: mol_type = other DNA
agcgagtactgtgagagcaa

SEQ ID NO: 277 - DNA, 20 bp, synthetic construct
  source 1..20: mol_type = other DNA
tcaccctcagcgagtactgt

SEQ ID NO: 278 - DNA, 20 bp, synthetic construct
  source 1..20: mol_type = other DNA
tcaggtcttttcttgttcac

SEQ ID NO: 279 - DNA, 20 bp, synthetic construct
  source 1..20: mol_type = other DNA
aatctttatcaggtcttttc

SEQ ID NO: 280 - DNA, 20 bp, synthetic construct
  source 1..20: mol_type = other DNA
ttctggttaatctttatcag

SEQ ID NO: 281 - DNA, 20 bp, synthetic construct
  source 1..20: mol_type = other DNA
ttgttttcttctggttaatc

SEQ ID NO: 282 - DNA, 20 bp, synthetic construct
  source 1..20: mol_type = other DNA
ttccctccttgttttcttct

SEQ ID NO: 283 - DNA, 20 bp, synthetic construct
  source 1..20: mol_type = other DNA
gcggttgtttccctccttgt

SEQ ID NO: 284 - DNA, 20 bp, synthetic construct
  source 1..20: mol_type = other DNA
tacaggctgcggttgtttcc

SEQ ID NO: 285 - DNA, 20 bp, synthetic construct
  source 1..20: mol_type = other DNA
gagcttgctacaggctgcgg

SEQ ID NO: 286 - DNA, 20 bp, synthetic construct
  source 1..20: mol_type = other DNA
gagttccagagcttgctaca

SEQ ID NO: 287 - DNA, 20 bp, synthetic construct
  source 1..20: mol_type = other DNA
cgactcctgagttccagagc

SEQ ID NO: 288 - DNA, 20 bp, synthetic construct
  source 1..20: mol_type = other DNA
cccggcccctagcgcgcgac

SEQ ID NO: 289 - DNA, 20 bp, synthetic construct
  source 1..20: mol_type = other DNA
gccccggccccggcccctag

SEQ ID NO: 290 - DNA, 20 bp, synthetic construct
  source 1..20: mol_type = other DNA
accacgccccggccccggcc

SEQ ID NO: 291 - DNA, 20 bp, synthetic construct
  source 1..20: mol_type = other DNA
ccgccccgaccacgccccgg

SEQ ID NO: 292 - DNA, 20 bp, synthetic construct
  source 1..20: mol_type = other DNA
ccccgggcccgccccgacca

SEQ ID NO: 293 - DNA, 20 bp, synthetic construct
  source 1..20: mol_type = other DNA
cgccccgggcccgcccccgg

SEQ ID NO: 294 - DNA, 20 bp, synthetic construct
  source 1..20: mol_type = other DNA
cgcagccccgccccgggccc

SEQ ID NO: 295 - DNA, 20 bp, synthetic construct
  source 1..20: mol_type = other DNA
accgcaaccgcagccccgcc

SEQ ID NO: 296 - DNA, 20 bp, synthetic construct
  source 1..20: mol_type = other DNA
gcgcaggcaccgcaaccgca

SEQ ID NO: 297 - DNA, 20 bp, synthetic construct
  source 1..20: mol_type = other DNA
ggcgcaggcaccgcaaccgc

SEQ ID NO: 298 - DNA, 20 bp, synthetic construct
  source 1..20: mol_type = other DNA
cgcctccgccgccgcgggcg

SEQ ID NO: 299 - DNA, 20 bp, synthetic construct
  source 1..20: mol_type = other DNA
accgcctgcgcctccgccgc

SEQ ID NO: 300 - DNA, 20 bp, synthetic construct
  source 1..20: mol_type = other DNA
cactcgccaccgcctgcgcc

SEQ ID NO: 301 - DNA, 20 bp, synthetic construct
  source 1..20: mol_type = other DNA
ccactcgccaccgcctgcgc

SEQ ID NO: 302 - DNA, 20 bp, synthetic construct
  source 1..20: mol_type = other DNA
ggtccccgggaaggagacag

SEQ ID NO: 303 - DNA, 20 bp, synthetic construct
  source 1..20: mol_type = other DNA
aacaactggtgcatggcaac

SEQ ID NO: 304 - DNA, 20 bp, synthetic construct
  source 1..20: mol_type = other DNA
gtttcagatgtactatcagc

SEQ ID NO: 305 - DNA, 20 bp, synthetic construct
  source 1..20: mol_type = other DNA
aaggtgaagttcatatcact

SEQ ID NO: 306 - DNA, 20 bp, synthetic construct
  source 1..20: mol_type = other DNA
ggtaacttcaaactcttggg

SEQ ID NO: 307 - DNA, 20 bp, synthetic construct
  source 1..20: mol_type = other DNA
ggttcatgagaggtttccca

SEQ ID NO: 308 - DNA, 20 bp, synthetic construct
  source 1..20: mol_type = other DNA
tactgaattgcttagtttta

SEQ ID NO: 309 - DNA, 20 bp, synthetic construct
  source 1..20: mol_type = other DNA
ctaacagaataagaaaaaaa

SEQ ID NO: 310 - DNA, 20 bp, synthetic construct
  source 1..20: mol_type = other DNA
gagcattagatgagtgcttt

SEQ ID NO: 311 - DNA, 20 bp, synthetic construct
  source 1..20: mol_type = other DNA
tgcattcctaagcaatgtgt

SEQ ID NO: 312 - DNA, 20 bp, synthetic construct
  source 1..20: mol_type = other DNA
tctaggccttcacattaatt

SEQ ID NO: 313 - DNA, 20 bp, synthetic construct
  source 1..20: mol_type = other DNA
cctgtctatgcctaggtgaa

SEQ ID NO: 314 - DNA, 20 bp, synthetic construct
  source 1..20: mol_type = other DNA
tagcacatacaattattaca

SEQ ID NO: 315 - DNA, 20 bp, synthetic construct
  source 1..20: mol_type = other DNA
gaggagaagaacataaacgc

SEQ ID NO: 316 - DNA, 20 bp, synthetic construct
  source 1..20: mol_type = other DNA
taccacaagtctggagccat

SEQ ID NO: 317 - DNA, 20 bp, synthetic construct
  source 1..20: mol_type = other DNA
gatactggattgttgaaact

SEQ ID NO: 318 - DNA, 20 bp, synthetic construct
  source 1..20: mol_type = other DNA
tagtatgactggagatttgg

SEQ ID NO: 319 - DNA, 20 bp, synthetic construct
  source 1..20: mol_type = other DNA
atcaaaaccccaaatgattt

SEQ ID NO: 320 - DNA, 20 bp, synthetic construct
  source 1..20: mol_type = other DNA
atccaaatgctccggagata

SEQ ID NO: 321 - DNA, 20 bp, synthetic construct
  source 1..20: mol_type = other DNA
tcgacatcactgcattccaa

SEQ ID NO: 322 - DNA, 20 bp, synthetic construct
  source 1..20: mol_type = other DNA
caacagctggagatggcggt

SEQ ID NO: 323 - DNA, 20 bp, synthetic construct
  source 1..20: mol_type = other DNA
atttgccacttaaagcaatc

SEQ ID NO: 324 - DNA, 20 bp, synthetic construct
  source 1..20: mol_type = other DNA
gtacctgttctgtctttgga

SEQ ID NO: 325 - DNA, 20 bp, synthetic construct
  source 1..20: mol_type = other DNA
caagaaaagttatttctcca

SEQ ID NO: 326 - DNA, 20 bp, synthetic construct
  source 1..20: mol_type = other DNA
gaaggatttctccatttaga

SEQ ID NO: 327 - DNA, 20 bp, synthetic construct
  source 1..20: mol_type = other DNA
ttacatctatagcaccactc

SEQ ID NO: 328 - DNA, 20 bp, synthetic construct
  source 1..20: mol_type = other DNA
tcactcccttttcagacaag

SEQ ID NO: 329 - DNA, 20 bp, synthetic construct
  source 1..20: mol_type = other DNA
agtttccatcaaagattaat

SEQ ID NO: 330 - DNA, 20 bp, synthetic construct
  source 1..20: mol_type = other DNA
atagtccatatgtgctgcga

SEQ ID NO: 331 - DNA, 20 bp, synthetic construct
  source 1..20: mol_type = other DNA
aactaagttctgtctgtgga

SEQ ID NO: 332 - DNA, 20 bp, synthetic construct
  source 1..20: mol_type = other DNA
caacacacactctatgaagt

SEQ ID NO: 333 - DNA, 20 bp, synthetic construct
  source 1..20: mol_type = other DNA
ttcctttccggattatatgt

SEQ ID NO: 334 - DNA, 20 bp, synthetic construct
  source 1..20: mol_type = other DNA
tttccattacaggaatcact

SEQ ID NO: 335 - DNA, 20 bp, synthetic construct
  source 1..20: mol_type = other DNA
atcagcctatatctatttcc

SEQ ID NO: 336 - DNA, 20 bp, synthetic construct
  source 1..20: mol_type = other DNA
tcaatgaccaggcggtcccc

SEQ ID NO: 337 - DNA, 20 bp, synthetic construct
  source 1..20: mol_type = other DNA
ctttttatggaaaaggaaaa

SEQ ID NO: 338 - DNA, 20 bp, synthetic construct
  source 1..20: mol_type = other DNA
tgtttccccaaaaatttctg

SEQ ID NO: 339 - DNA, 20 bp, synthetic construct
  source 1..20: mol_type = other DNA
agatatccactcgccaccgc

SEQ ID NO: 340 - DNA, 20 bp, synthetic construct
  source 1..20: mol_type = other DNA
ccggccccggccccggcccc

SEQ ID NO: 341 - DNA, 20 bp, synthetic construct
  source 1..20: mol_type = other DNA
cccggccccggccccggccc

SEQ ID NO: 342 - DNA, 20 bp, synthetic construct
  source 1..20: mol_type = other DNA
ccccggccccggccccggcc

SEQ ID NO: 343 - DNA, 20 bp, synthetic construct
  source 1..20: mol_type = other DNA
gccccggccccggccccggc

SEQ ID NO: 344 - DNA, 20 bp, synthetic construct
  source 1..20: mol_type = other DNA
ggccccggccccggccccgg

SEQ ID NO: 345 - DNA, 20 bp, synthetic construct
  source 1..20: mol_type = other DNA
cggccccggccccggccccg

SEQ ID NO: 346 - DNA, 19 bp, synthetic construct
  source 1..19: mol_type = other DNA
cggccccggccccggcccc

SEQ ID NO: 347 - DNA, 19 bp, synthetic construct
  source 1..19: mol_type = other DNA
ccggccccggccccggccc

SEQ ID NO: 348 - DNA, 19 bp, synthetic construct
  source 1..19: mol_type = other DNA
cccggccccggccccggcc

SEQ ID NO: 349 - DNA, 19 bp, synthetic construct
  source 1..19: mol_type = other DNA
ccccggccccggccccggc

SEQ ID NO: 350 - DNA, 19 bp, synthetic construct
  source 1..19: mol_type = other DNA
gccccggccccggccccgg

SEQ ID NO: 351 - DNA, 19 bp, synthetic construct
  source 1..19: mol_type = other DNA
ggccccggccccggccccg

SEQ ID NO: 352 - DNA, 18 bp, synthetic construct
  source 1..18: mol_type = other DNA
ggccccggccccggcccc

SEQ ID NO: 353 - DNA, 18 bp, synthetic construct
  source 1..18: mol_type = other DNA
cggccccggccccggccc

SEQ ID NO: 354 - DNA, 18 bp, synthetic construct
  source 1..18: mol_type = other DNA
ccggccccggccccggcc

SEQ ID NO: 355 - DNA, 18 bp, synthetic construct
  source 1..18: mol_type = other DNA
cccggccccggccccggc

SEQ ID NO: 356 - DNA, 18 bp, synthetic construct
  source 1..18: mol_type = other DNA
ccccggccccggccccgg

SEQ ID NO: 357 - DNA, 18 bp, synthetic construct
  source 1..18: mol_type = other DNA
gccccggccccggccccg

SEQ ID NO: 358 - DNA, 17 bp, synthetic construct
  source 1..17: mol_type = other DNA
gccccggccccggcccc

SEQ ID NO: 359 - DNA, 17 bp, synthetic construct
  source 1..17: mol_type = other DNA
ggccccggccccggccc

SEQ ID NO: 360 - DNA, 17 bp, synthetic construct
  source 1..17: mol_type = other DNA
cggccccggccccggcc

SEQ ID NO: 361 - DNA, 17 bp, synthetic construct
  source 1..17: mol_type = other DNA
ccggccccggccccggc

SEQ ID NO: 362 - DNA, 17 bp, synthetic construct
  source 1..17: mol_type = other DNA
cccggccccggccccgg

SEQ ID NO: 363 - DNA, 17 bp, synthetic construct
  source 1..17: mol_type = other DNA
ccccggccccggccccg

SEQ ID NO: 364 - DNA, 16 bp, synthetic construct
  source 1..16: mol_type = other DNA
ccccggccccggcccc

SEQ ID NO: 365 - DNA, 16 bp, synthetic construct
  source 1..16: mol_type = other DNA
gccccggccccggccc

SEQ ID NO: 366 - DNA, 16 bp, synthetic construct
  source 1..16: mol_type = other DNA
ggccccggccccggcc

SEQ ID NO: 367 - DNA, 16 bp, synthetic construct
  source 1..16: mol_type = other DNA
cggccccggccccggc

SEQ ID NO: 368 - DNA, 16 bp, synthetic construct
  source 1..16: mol_type = other DNA
ccggccccggccccgg

SEQ ID NO: 369 - DNA, 16 bp, synthetic construct
  source 1..16: mol_type = other DNA
cccggccccggccccg

SEQ ID NO: 370 - DNA, 20 bp, synthetic construct
  source 1..20: mol_type = other DNA
  modified_base 1: mod_base = OTHER; note = 2'-MOE guanosine 3'-phosphorothioate
  modified_base 2: mod_base = OTHER; note = 2'-MOE adenosine 3'-phosphodiester
  modified_base 3..4: mod_base = OTHER; note = 2'-MOE 5-methylcytidine 3'-phosphodiester
  modified_base 5: mod_base = OTHER; note = 2'-MOE guanosine 3'-phosphorothioate
  modified_base 6: mod_base = OTHER; note = 5-methyl-2'-deoxycytidine 3'-phosphorothioate
  modified_base 7..8: mod_base = OTHER; note = 2'-deoxythymidine 3'-phosphorothioate
  modified_base 9: mod_base = OTHER; note = 2'-deoxyguanosine 3'-phosphorothioate
  modified_base 10: mod_base = OTHER; note = 2'-deoxyadenosine 3'-phosphorothioate
  modified_base 11: mod_base = OTHER; note = 2'-deoxyguanosine 3'-phosphorothioate
  modified_base 12..14: mod_base = OTHER; note = 2'-deoxythymidine 3'-phosphorothioate
  modified_base 15: mod_base = OTHER; note = 2'-deoxyguanosine 3'-phosphorothioate
  modified_base 16..17: mod_base = OTHER; note = 2'-MOE 5-methylcytidine 3'-phosphodiester
  modified_base 18: mod_base = OTHER; note = 2'-MOE adenosine 3'-phosphodiester
  modified_base 19: mod_base = OTHER; note = 2'-MOE 5-methylcytidine 3'-phosphorothioate
  modified_base 20: mod_base = OTHER; note = 2'-MOE adenosine
gaccgcttgagtttgccaca